(19) United States
(12) Patent Application Publication
(10) Pub. No.: US 2025/0127598 A1
(43) Pub. Date: Apr. 24, 2025

(54) INTRAORAL SCANNER

(71) Applicant: Align Technology, Inc., San Jose, CA (US)

(72) Inventors: Yossef Y. Atiya, Maccabim (IL); Ido Tishel, Kfar Bilu (IL); Avi Kopelman, Palo Alto, CA (US); Ofer Saphier, Rechovot (IL); Nadav Fain, Petach Tikva (IL); Eddy Pincu, Rishon LeZion (IL); Tal Verker, Ofra (IL); Nir Makmel, Ramat Gan (IL); Asaf Pellman, Bet-Yehoshua (IL)

(21) Appl. No.: 18/913,010

(22) Filed: Oct. 11, 2024

Related U.S. Application Data
(60) Provisional application No. 63/592,816, filed on Oct. 24, 2023.
(60) Provisional application No. 63/592,819, filed on Oct. 24, 2023.

(51) Int. Cl. A61C 9/00

(52) U.S. Cl. CPC A61C 9/0053

(57) ABSTRACT

An intraoral scanner includes a probe housing disposed at a distal end of an elongate wand. The intraoral scanner further includes a window structure coupled to the probe housing. The window structure and the probe housing form an interior volume. The window structure has a concave transverse cross section. The intraoral scanner further includes optical components disposed within the interior volume. The optical components include a first camera having a first orientation and a second camera having a second orientation that is different than the first orientation. The first camera and the second camera are to capture images of dental sites.

### RELATED APPLICATIONS

This application claims benefit of U.S. Provisional Application No. 63/592,816, filed Oct. 24, 2023, and U.S. Provisional Application No. 63/592,819, filed Oct. 24, 2023, the contents of which are incorporated by reference in their entirety.

### TECHNICAL FIELD

Embodiments of the present disclosure relate to the field of dentistry and, in particular, to an intraoral scanner.

### BACKGROUND

A dental site of a patient is to be measured accurately and studied carefully so that dental procedures can be performed.

### SUMMARY

In a first implementation, an intraoral scanner includes: a probe housing disposed at a distal end of an elongate wand; a window structure coupled to the probe housing, the window structure and the probe housing forming an interior volume, wherein the window structure has a concave transverse cross section; and a plurality of optical components disposed within the interior volume, wherein the plurality of optical components comprise a first camera having a first orientation and a second camera having a second orientation, wherein the first camera and the second camera are to capture images of dental sites.

A second implementation may further extend the first implementation. In the second implementation, the plurality of optical components comprises a first pattern projector, a second pattern projector, and a camera disposed between the first pattern projector and the second pattern projector.

A third implementation may further extend the first or second implementations. In the third implementation, the camera, the first pattern projector and the second pattern projector are disposed within 5 millimeters of a foremost end of the probe housing.

A fourth implementation may further extend any of the first through third implementations. In the fourth implementation, each of the first pattern projector and the second pattern projector has a diameter of less than 4 millimeters and a height of less than 4.5 millimeters.

A fifth implementation may further extend any of the first through fourth implementations. In the fifth implementation, each of the first pattern projector and the second pattern projector has a diameter of about 2 millimeters and a height of about 3 millimeters.

A sixth implementation may further extend any of the first through fifth implementations. In the sixth implementation, the probe housing has a height of less than 10 millimeters and a width of less than 20 millimeters.

A seventh implementation may further extend any of the first through sixth implementations. In the seventh implementation, the probe housing has a height of about 7.5 to about 8.5 millimeters and a width of about 16 to about 17 millimeters.

An eighth implementation may further extend any of the first through seventh implementations. In the eighth implementation, the first orientation of the first camera and the second orientation of the second camera are configured to cause about 20% to about 40% overlap of image capture between the first camera and the second camera.

A nineth implementation may further extend any of the first through eighth implementations. In the nineth implementation, the first camera and the second camera are to capture images, wherein the images are to be used to perform model building via at least one of a correspondence algorithm or a trained machine learning model.

A tenth implementation may further extend any of the first through nineth implementations. In the tenth implementation, one or more of the plurality of optical components are configured to capture images of rearmost teeth in a mouth of a patient.

An eleventh implementation may further extend any of the first through tenth implementations. In the eleventh implementation, the first camera is to perform image capturing in the first orientation of a first side of the dental sites and the second camera is to perform image capturing in the second orientation of a second side of the dental sites to perform teeth wrapping imaging.

A twelfth implementation may further extend any of the first through eleventh implementations. In the twelfth implementation, a first central axis of the first camera is orthogonal to a first portion of the window structure, and wherein a second central axis of the second camera is orthogonal to a second portion of the window structure.

In a thirteenth implementation, an intraoral scanner includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, the plurality of optical components being bonded directly to the window structure.

A fourteenth implementation may further extend the thirteenth implementation. In the fourteenth implementation, the plurality of optical components comprises cameras and projectors.

A fifteenth implementation may further extend the thirteenth or fourteenth implementations. In the fifteenth implementation, the plurality of optical components are bonded directly to the window structure to provide drift-free retention of the plurality of optical components.

A sixteenth implementation may further extend any of the thirteenth through fifteenth implementations. In the sixteenth implementation, the plurality of optical components are bonded directly to the window structure via adhesive that is optically permeable.

In a seventeenth implementation, an intraoral scanner includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, wherein the window structure comprises a first distal portion disposed at a first orientation and a second distal portion disposed at a second orientation that is at an angle to the first orientation, wherein the window structure is configured to block back reflection crosstalk by the plurality of optical components.

An eighteenth implementation may further extend the seventeenth implementation. In the eighteenth implementation, the plurality of optical components comprises cameras and projectors.

A nineteenth implementation may further extend the seventeenth or eighteenth implementations. In the nineteenth implementation, the window structure is a single piece of glass formed via glass molding.

A twentieth implementation may further extend any of the seventeenth through nineteenth implementations. In the twentieth implementation, the probe housing is wrapped by a heat sink metal structure that is in thermal contact with the probe housing.

A twenty-first implementation may further extend any of the seventeenth through twentieth implementations. In the twenty-first implementation, a heating element is disposed between two or more of the plurality of optical components to heat the intraoral scanner.

A twenty-second implementation may further extend any of the seventeenth through twenty-first implementations. In the twenty-second implementation, the intraoral scanner further comprises a sleeve that includes an optical window, wherein the sleeve is configured to be removably disposed over the probe housing, wherein the optical window is configured to substantially align with the window structure.

A twenty-third implementation may further extend any of the seventeenth through twenty-second implementations. In the twenty-third implementation, the optical window of the sleeve comprises a first portion that approximately aligns with the first distal portion of the window structure, a second portion that approximately aligns with the second distal portion of the window structure, and a third portion that approximately aligns with a third portion of the window structure, the third portion of the window structure being disposed between the first distal portion and the second distal portion.

A twenty-fourth implementation may further extend any of the seventeenth through twenty-third implementations. In the twenty-fourth implementation, the window structure has a concave transverse cross section.

A twenty-fifth implementation may further extend any of the seventeenth through twenty-fourth implementations. In the twenty-fifth implementation, a first optical component of the plurality of optical components has a first axis that is orthogonal to the first distal portion of the window structure, wherein a second optical component of the plurality of optical components has a second axis that is orthogonal to the second distal portion of the window structure, and wherein a third optical component of the plurality of optical components has a third axis that is orthogonal to a third portion of the window structure, the third portion of the window structure being disposed between the first distal portion and the second distal portion.

A twenty-sixth implementation may further extend any of the seventeenth through twenty-fifth implementations. In the twenty-sixth implementation, the first optical component comprises a first structured light projector, wherein the second optical component comprises a camera, and wherein the third optical component comprises a second structured light projector.

A twenty-seventh implementation may further extend any of the seventeenth through twenty-sixth implementations. In the twenty-seventh implementation, the first optical component comprises a first camera, wherein the second optical component comprises a structured light projector, and wherein the third optical component comprises a second camera.

In a twenty-eighth implementation, an intraoral scanner includes: a probe housing disposed at a distal end of an elongate wand, the probe housing forming an interior volume; and a distributed projector disposed in the interior volume, the distributed projector comprising: a diode module comprising a laser diode configured to emit a beam of light; and a lens module comprising a first pattern generating optical element configured to generate structured light based on at least a first portion of the beam of light, the lens module being disposed at least a threshold distance from the diode module.

A twenty-nineth implementation may further extend the twenty-eighth implementation. In the twenty-nineth implementation, the first pattern generating optical element is at least one of a multi lens array (MLA) or a diffractive optical element (DOE).

A thirtieth implementation may further extend the twenty-eighth or twenty-nineth implementations. In the thirtieth implementation, the lens module further comprises a first folding prism configured to deflect the at least a first portion of the beam of light to the first pattern generating optical element.

A thirty-first implementation may further extend any of the twenty-eighth through thirtieth implementations. In the thirty-first implementation: the lens module further comprises a second folding prism and a second pattern generating optical element; the first folding prism is configured to transmit a second portion of the beam of light to the second folding prism; and the second folding prism is configured to deflect the second portion of the beam of light to the second pattern generating optical element.

A thirty-second implementation may further extend any of the twenty-eighth through thirty-first implementations. In the thirty-second implementation, the lens module further comprises a relay lens configured to focus the beam of light.

A thirty-third implementation may further extend any of the twenty-eighth through thirty-second implementations. In the thirty-third implementation, the relay lens is disposed between the diode module and a folding prism of the lens module.

A thirty-fourth implementation may further extend any of the twenty-eighth through thirty-third implementations. In the thirty-fourth implementation, the relay lens is disposed between a first folding prism of the lens module and the first pattern generating optical element.

A thirty-fifth implementation may further extend any of the twenty-eighth through thirty-fourth implementations. In the thirty-fifth implementation, the relay lens is further configured to deflect at least the first portion of the beam of light to the first pattern generating optical element.

A thirty-sixth implementation may further extend any of the twenty-eighth through thirty-fifth implementations. In the thirty-sixth implementation, the diode module further comprises focusing optics configured to focus the beam of light, the focusing optics being disposed between the laser diode and the lens module.

A thirty-seventh implementation may further extend any of the twenty-eighth through thirty-sixth implementations. In the thirty-seventh implementation, the first pattern generating optical element is disposed on a window structure of the intraoral scanner.

A thirty-eighth implementation may further extend any of the twenty-eighth through thirty-seventh implementations. In the thirty-eighth implementation, the intraoral scanner further comprises a plurality of cameras disposed in a camera portion of the intraoral scanner, wherein the camera portion of the intraoral scanner is disposed between the diode module and the lens module.

A thirty-nineth implementation may further extend any of the twenty-eighth through thirty-eighth implementations. In the thirty-nineth implementation, the beam of light emitted from the diode module is to pass between two or more cameras and is to be received by the lens module.

A fortieth implementation may further extend any of the twenty-eighth through thirty-nineth implementations. In the fortieth implementation, the intraoral scanner further comprises a first camera and a second camera, the first camera and the second camera being disposed in a distal portion of the intraoral scanner, wherein the lens module is disposed between the first camera and the second camera.

A forty-first implementation may further extend any of the twenty-eighth through fortieth implementations. In the forty-first implementation, the intraoral scanner further comprises: a window coupled to the probe housing, wherein the lens module is disposed at or proximate to a first distal end of the window and the diode module is disposed at or proximate to a second distal end of the window.

A forty-second implementation may further extend any of the twenty-eighth through forty-first implementations. In the forty-second implementation, the intraoral scanner further comprises a window coupled to the probe housing, wherein the window covers the lens module and does not cover the diode module.

A forty-third implementation may further extend any of the twenty-eighth through forty-second implementations. In the forty-third implementation, the lens module is separated from the diode module by air, an optical fiber, or a light guide.

In a forty-fourth implementation, an intraoral scanner includes: a probe housing disposed at a distal end of an elongate wand, the probe housing forming an interior volume; and a distributed projector disposed in the interior volume, the distributed projector comprising: a diode module configured to emit a first beam of light at a first wavelength; and a first prism component comprising: a first beam splitter configured to deflect a first portion of the first beam of light and transmit a second portion of the first beam of light; and a second beam splitter configured to deflect the second portion of the first beam of light.

An forty-fifth implementation may further extend the forty-fourth implementation. In the eighteenth implementation, the intraoral scanner further comprises: a lens module disposed at least a threshold distance from the diode module, the lens module comprising: a relay lens configured to focus the first portion of the first beam of light; a folding prism configured to deflect the first portion of the first beam of light; and a pattern generating optical element configured to generate first structured light based on the first portion of the first beam of light.

A forty-sixth implementation may further extend the forty-fourth or forty-fifth implementations. In the nineteenth implementation, the diode module is further configured to emit a second beam of light at a second wavelength.

A forty-seventh implementation may further extend any of the forty-fourth through forty-sixth implementations. In the twentieth implementation, the intraoral scanner further comprises: a second prism component comprising: a third beam splitter configured to deflect a first portion of the second beam of light and transmit a second portion of the second beam of light; and a fourth beam splitter configured to deflect the second portion of the second beam of light.

A forty-eighth implementation may further extend any of the forty-fourth through forty-seventh implementations. In the twenty-first the first beam splitter and the second beam splitter are further configured to transmit the second beam of light, wherein the first prism component further comprises: a third beam splitter configured to deflect a first portion of the second beam of light and transmit a second portion of the second beam of light; and a fourth beam splitter configured to deflect the second portion of the second beam of light.

In a forty-nineth implementation, distributed projector is of an intraoral scanner, the distributed projector includes: a diode module comprising a laser diode configured to emit a beam of light; and a lens module disposed at least a threshold distance from the diode module, the lens module comprising: a folding prism configured to deflect at least a first portion of the beam of light; and a pattern generating optical element configured to generate, based on the at least a first portion of the beam of light, structured light to illuminate at least a portion of a mouth of a patient.

A fiftieth implementation may further extend the forty-nineth implementation. In the twenty-third implementation, the diode module further comprises focusing optics configured to focus the beam of light.

A fifty-first implementation may further extend the forty-nineth or fiftieth implementations. In the twenty-fourth implementation, the lens module further comprises a relay lens configured to focus the beam of light.

In a fifty-second implementation, an intraoral scanner includes: a probe housing disposed at a distal end of an elongate wand, the probe housing forming an interior volume; a non-distributed projector disposed in the interior volume; and a distributed projector disposed in the interior volume, the distributed projector comprising a diode module and a lens module.

A fifty-third implementation may further extend the fifty-second implementation. In the fifty-third implementation: the non-distributed projector comprising components configured to emit a first beam of light and generate first structured light; the components are less than a threshold distance from each other; the diode module is configured to emit a second beam of light; the lens module is configured to generate second structured light based on at least a portion of the second beam of light; and the lens module is disposed greater than the threshold distance from the diode module.

A fifty-fourth implementation may further extend the fifty-second or fifty-third implementations. In the fifty-fourth implementation, the lens module is disposed at a distal end of the probe housing.

A fifty-fifth implementation may further extend any of the fifty-second through fifty-fourth implementations. In the fifty-fifth implementation, the distal end of the probe housing has an angled tip that houses the lens module.

In a fifty-sixth implementation, a hybrid intraoral scanner includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view of about 70 to about 90 degrees; and one or more non-object-facing imaging devices disposed within the interior volume in a rear portion of the probe housing, wherein the one or more non-object-facing imaging devices are configured to capture respective images via respective imaging axes that reflect to pass through the window structure, and wherein the one or more object-facing imaging devices have a second field of view of about 10 to about 30 degrees.

A fifty-seventh implementation may further extend the fifty-sixth implementation. In the fifty-seventh implementation: the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in the rear portion of the probe housing, wherein the one or more non-object-facing projectors provide respective light via respective projector axes that reflect to pass through the window structure.

A fifty-eighth implementation may further extend the fifty-sixth or fifty-seventh implementations. In the fifty-eighth implementation: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors.

A fifty-nineth implementation may further extend any of the fifty-sixth through fifty-eighth implementations. In the fifty-nineth implementation, the hybrid intraoral scanner further includes a fold mirror disposed at a distal end of the probe housing, wherein the fold mirror is configured to reflect the respective imaging axes and the respective projector axes to provide structured light 3D capture of a distal portion of a user mouth, and wherein the one or more non-object-facing imaging devices are oriented substantially parallel to the window structure.

A sixtieth implementation may further extend any of the fifty-sixth through fifty-nineth implementations. In the sixtieth implementation, the hybrid intraoral scanner further includes a prism disposed at a distal end of the probe housing, wherein the prism is configured to reflect the respective imaging axes and the respective projector axes to provide structured light 3D capture of a distal portion of a user mouth, wherein the prism has a fold angle of about 25 degrees to about 40 degrees, and wherein the one or more non-object-facing imaging devices are oriented substantially parallel to the window structure.

A sixty-first implementation may further extend any of the fifty-sixth through sixtieth implementations. In the sixty-first implementation, the hybrid intraoral scanner further includes a dichroic filter, wherein the one or more non-object-facing imaging devices comprise one or more upward-facing cameras that are oriented away from the window structure and an optical coherence tomography (OCT) channel that is oriented substantially parallel to the window structure, wherein the one or more upward-facing cameras are oriented upward and are configured to combine coaxially with a forward pointing OCT channel via the dichroic filter.

A sixty-second implementation may further extend any of the fifty-sixth through sixty-first implementations. In the sixty-second implementation, a first camera of the one or more object-facing imaging devices is configured to provide a viewfinder associated with moving the hybrid intraoral scanner.

A sixty-third implementation may further extend any of the fifty-sixth through sixty-second implementations. In the sixty-third implementation, the one or more object-facing imaging devices and the one or more non-object-facing imaging devices are configured to provide imaging of one or more of a three-dimensional (3D) surface, subgingival, occlusal caries, proximal caries, or periodontal pockets.

A sixty-fourth implementation may further extend any of the fifty-sixth through sixty-third implementations. In the sixty-fourth implementation, the one or more object-facing imaging devices and the one or more non-object-facing imaging devices are configured to provide structured near-infrared (NIRI) images to provide shallow 3D viewing of hard tissue lesions.

A sixty-fifth implementation may further extend any of the fifty-sixth through sixty-fourth implementations. In the sixty-fifth implementation, the one or more object-facing imaging devices and the one or more object-facing projectors are configured to perform surface 3D capture.

A sixty-sixth implementation may further extend any of the fifty-sixth through sixty-fifth implementations. In the sixty-sixth implementation, the hybrid intraoral scanner further comprises an array of object-facing LEDs associated with two-dimensional color capture.

A sixty-seventh implementation may further extend any of the fifty-sixth through sixty-sixth implementations. In the sixty-seventh implementation, the one or more non-object-facing projectors comprise one or more of a rear NIRI structured light or NIRI collimated projector for subsurface 3D capture.

A sixty-eighth implementation may further extend any of the fifty-sixth through sixty-seventh implementations. In the sixty-eighth implementation, the one or more non-object-facing imaging devices comprise an OCT imaging channel coaxial configured for surface and sub-surface 3D structure capture.

A sixty-nineth implementation may further extend any of the fifty-sixth through sixty-eighth implementations. In the sixty-nineth implementation, the one or more non-object-facing imaging devices comprises an endoscopic OCT device inserted between the one or more object-facing imaging devices and the one or more object-facing projectors, wherein the endoscopic OCT device comprises a built-in rotating fold prism configured to provide transverse line scanning.

A seventieth implementation may further extend any of the fifty-sixth through sixty-nineth implementations. In the seventieth implementation, image data from the one or more non-object-facing imaging devices and the one or more object-facing imaging devices is to be combined to provide a third field of view that is greater than at least one of the first field of view or the second field of view.

A seventy-first implementation may further extend any of the fifty-sixth through seventieth implementations. In the seventy-first implementation,
the hybrid intraoral scanner further comprises a collimator, a scan mirror, and a fold mirror, wherein the one or more non-object-facing imaging devices comprise an OCT measuring arm fiber configured to provide light that is to pass through the collimator, reflect off the scan mirror, and reflect off the fold mirror to pass through the window structure.

In a seventy-second implementation, a method includes: identifying first image data from one or more object-facing imaging devices at a first field of view of about 70 to about 90 degrees, the one or more object-facing imaging devices being disposed within an interior volume formed by a probe housing and a window structure of a hybrid intraoral scanner, the one or more object-facing imaging devices being disposed proximate the window structure; identifying second image data from one or more non-object-facing imaging devices at a second field of view of about 10 to about 30 degrees, the one or more non-object-facing imaging devices being disposed within the interior volume in a rear portion of the probe housing; and combining the first image data and the second image data.

A seventy-third implementation may further extend the seventy-second implementation. In the seventy-third implementation, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in a rear portion of the probe housing, wherein the one or more non-object-facing projectors provide respective light via respective projector axes that reflect to pass through the window structure.

A seventy-fourth implementation may further extend the seventy-second or seventy-third implementations. In the seventy-fourth implementation: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors.

In a seventy-fifth implementation, a hybrid intraoral scanner includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view; and an optical coherence tomography (OCT) imaging channel disposed within the interior volume in a rear portion of the probe housing, wherein the OCT imaging channel is configured to capture respective images via a respective imaging axis that reflects to pass through the window structure, and wherein the OCT imaging channel has a second field of view that is different from the first field of view.

A seventy-sixth implementation may further extend the seventy-fifth implementation. In the seventy-sixth implementation, the hybrid intraoral scanner further includes one or more object-facing projectors disposed within the interior volume proximate the window structure, wherein the one or more object-facing projectors provide corresponding light via corresponding projector axes that directly pass through the window structure.

A seventy-seventh implementation may further extend the seventy-fifth or seventy-sixth implementations. In the seventy-seventh implementation the one or more object-facing devices and the one or more object-facing projectors are closer to a distal tip of the probe housing than the OCT imaging channel.

In a seventy-eighth implementation, an intraoral scanner includes: a processing device configured to: receive a first image of an object from a first camera of the intraoral scanner, the first camera having a first static focal distance; determine a more sharp portion of the first image and a less sharp portion of the first image, the less sharp portion corresponding to a region of the object; for the less sharp portion of the first image, determine a more sharp portion of a second image corresponding to the region of the object, the second image being from a second camera of the intraoral scanner, the second camera having a second static focal distance that is different from the first static focal distance of the first camera; generate a combined image using the more sharp portion of the first image and the more sharp portion of the second image; and cause, based on the combined image, an action associated with a mouth of a user.

A seventy-nineth implementation may further extend the seventy-eighth implementation. In the seventy-nineth implementation, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface.

An eightieth implementation may further extend the seventy-eighth or seventy-nineth implementations. In the eightieth implementation: the processing device is further configured to receive a third image of the object from a third camera of the intraoral scanner; the third camera has a third static focal distance that is different from the first static focal distance of the first camera and the second static focal distance of the second camera; and the processing device is to generate the combined image further based on the third image.

An eighty-first implementation may further extend any of the seventy-eighth through eightieth implementations. In the eighty-first implementation: the first camera is proximate a distal end of a tip of the intraoral scanner; the third camera is further from the distal end of a tip of the intraoral scanner than the first camera; the second camera is disposed between the first camera and the third camera; the first static focal distance of the first camera is less than the second static focal distance of the second camera; and the second static focal distance of the second camera is less than the third static focal distance of the third camera.

An eighty-second implementation may further extend any of the seventy-eighth through eighty-first implementations. In the eighty-second implementation, the combined image has an increased sharpness than the sharp portion of the first image and the sharp portion of the second image.

An eighty-third implementation may further extend any of the seventy-eighth through eighty-second implementations. In the eighty-third implementation, the first image of the object being captured by the first camera and the second image of the object being captured by the second camera in a similar location, in a similar angle, and at a similar brightness level.

An eighty-fourth implementation may further extend any of the seventy-eighth through eighty-third implementations. In the eighty-fourth implementation, the first camera and second camera capture images of the mouth of the user responsive to a light source of the intraoral scanner providing white light or near infrared light into the mouth of the user.

An eighty-fifth implementation may further extend any of the seventy-eighth through eighty-fourth implementations. In the eighty-fifth implementation, to generate the combined image, the processing device is to smooth the more sharp portion of the first image and the more sharp portion of the second image.

In an eighty-sixth implementation, an intraoral scanner includes: a processing device configured to: receive a first image of an object from a first camera of the intraoral scanner, the first camera having a first static focal distance; receive a second image of the object from a second camera of the intraoral scanner, the second camera having a second static focal distance that is different from the first static focal distance; determine a first sharp portion of the first image corresponding to a first region of the object; determine a second sharp portion of the second image corresponding to the first region of the object; generate a combined image using the first sharp portion of the first image and the second sharp portion of the second image; and cause, based on the combined image, an action associated with a mouth of a user.

An eighty-seventh implementation may further extend the eighty-sixth implementation, in the eighty-seventh implementation, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface.

### BRIEF DESCRIPTION OF THE DRAWINGS

Embodiments of the present disclosure are illustrated by way of example, and not by way of limitation, in the figures of the accompanying drawings.

FIG. 1 illustrates a system for performing intraoral scanning and/or generating a virtual three-dimensional (3D) model of a dental site, according to certain embodiments.

FIG. 2A is a schematic illustration of a handheld intraoral scanner with a plurality of cameras disposed within a probe at a distal end of the intraoral scanner, according to certain embodiments.

FIGS. 2B-2C include schematic illustrations of positioning configurations for cameras and structured light projectors of an intraoral scanner, according to certain embodiments.

FIG. 2D is a chart depicting a plurality of different configurations for the position of structured light projectors and cameras in a probe of an intraoral scanner, according to certain embodiments.

FIGS. 3A-L illustrate components of intraoral scanners, according to certain embodiments.

FIGS. 4A-0 illustrate components of intraoral scanners, according to certain embodiments.

FIGS. 5A-F illustrate intraoral scanners, according to certain embodiments.

FIG. 6A illustrates an intraoral scanner, according to certain embodiments.

FIGS. 6B-C are flow diagrams of methods associated with intraoral scanners, according to certain embodiments.

FIG. 7 illustrates a block diagram of an example computing device, according to certain embodiments.

### DETAILED DESCRIPTION

Described herein are devices, systems, and methods associated with intraoral scanners (e.g., multi-structural triangulation 3D capture intraoral scanner, intraoral scanners having ultra-low tip profiles, intraoral scanner distributed projectors, hybrid approach of tip-mounted and back-mounted pattern projectors, etc.).

A dental site of a patient is to be measured accurately and studied carefully so that dental procedures can be performed. For example, in prosthodontic procedures designed to implant a dental prosthesis in the oral cavity, the dental site at which the prosthesis is to be implanted in many cases should be measured accurately and studied carefully, so that a prosthesis such as a crown, denture or bridge, for example, can be properly designed and dimensioned to fit in place. A good fit enables mechanical stresses to be properly transmitted between the prosthesis and the jaw, and to prevent infection of the gums via the interface between the prosthesis and the dental site, for example. Some procedures also call for removable prosthetics to be fabricated to replace one or more missing teeth, such as a partial or full denture, in which case the surface contours of the areas where the teeth are missing need to be reproduced accurately so that the resulting prosthetic fits over the edentulous region with even pressure on the soft tissues.

In some practices, the dental site is prepared by a dental practitioner, and a positive physical model of the dental site is constructed using known methods. Alternatively, the dental site may be scanned to provide 3D data of the dental site. In either case, the virtual or real model of the dental site is sent to the dental lab, which manufactures the prosthesis based on the model. However, if the model is deficient or undefined in certain areas, or if the preparation was not optimally configured for receiving the prosthesis, the design of the prosthesis may be less than optimal. For example, if the insertion path implied by the preparation for a closely-fitting coping would result in the prosthesis colliding with adjacent teeth, the coping geometry has to be altered to avoid the collision, which may result in the coping design being less optimal. Further, if the area of the preparation containing a finish line lacks definition, it may not be possible to properly determine the finish line and thus the lower edge of the coping may not be properly designed. Indeed, in some circumstances, the model is rejected and the dental practitioner then re-scans the dental site, or reworks the preparation, so that a suitable prosthesis may be produced.

In orthodontic procedures it can be important to provide a model of one or both jaws. Where such orthodontic procedures are designed virtually, a virtual model of the oral cavity is also beneficial. Such a virtual model may be obtained by scanning the oral cavity directly, or by producing a physical model of the dentition, and then scanning the model with a suitable scanner.

Thus, in both prosthodontic and orthodontic procedures, obtaining a 3D model of a dental site in the oral cavity is an initial procedure that is performed. When the 3D model is a virtual model, the more complete and accurate the scans of the dental site are, the higher the quality of the virtual model, and thus the greater the ability to design an optimal prosthesis or orthodontic treatment appliance(s).

A scanner may have multiple projectors and multiple cameras. Each projector may project a pattern of light on a dental site in the field of view of at least one camera. The cameras capture images of the patterns of light on the dental site. The images are used to generate a 3D model of the dental site.

Conventional cameras and projectors are large and cause conventional scanners to be wide and tall which causes inefficient and slow scanner maneuvering. Conventional cameras and projectors may drift within conventional scanners causing inaccurate image capture and modeling. Conventional scanners may have back reflection crosstalk causing inaccurate image capture and modeling. Back reflection crosstalk may be caused by at least a portion of light from projectors being reflected (e.g., back reflected) from the window structure to the cameras causing crosstalk of projector light that has not passed through the window structure being captured by cameras (e.g., instead of or in addition to cameras capturing light that passed through the window structure onto dental sites). Conventional scanners may not reach certain portions of a mouth (e.g., rearmost molars, etc.). Conventional scanners may have components (e.g., heaters) that block image capture or reduce quality of image capture.

A scanner may use at least two cameras that overlap to capture images to generate a 3D model of the dental site. A projector is to illuminate the dental site where the cameras are capturing images. To generate a 3D model of the rearmost molars, two cameras and a projector are located at a tip of the scanner. This causes the scanner to have an increased width which causes inefficient and slow scanner maneuvering. This may also cause conventional scanners to not reach certain portions of a mouth (e.g., rearmost molars, etc.).

The devices, systems, and methods of the present disclosure overcome some or all of these challenges.

An intraoral scanner includes an elongate wand that is used to scan inside a mouth (e.g., scan dental arches) of a patient. The intraoral scanner includes a probe housing disposed at a distal end of the elongate wand. At least a portion of the probe housing is to be inserted into a mouth of a patient for scanning. A window structure is coupled to the probe housing. The window structure and the probe housing form an interior volume. Optical components (e.g., cameras, projectors) are disposed proximate the window structure in the interior volume.

In some embodiments, the optical components include a first camera disposed at a first angle and a second camera disposed at a second angle. A first central axis of the first camera intersects with a second central axis of the second camera below the probe housing. The first camera and the second camera may be configured to capture images of one or more locations below the probe housing to perform teeth wrapping imaging. Teeth wrapping imaging may include performing image capturing (e.g., via a first camera) in a first orientation of a first side of dental sites and performing image capturing (e.g., via a second camera) in a second orientation of a second side of dental sites at the same time or substantially the same time. The captured images may wrap (e.g., be of different sides) of the dental sites.

In some embodiments, the optical components are bonded directly to the window structure.

In some embodiments, the window structure (e.g., a segmented front glass optical window) includes a first distal portion disposed at a first orientation and a second distal portion at a second orientation that is at an angle to the first orientation. The window structure is configured to block back reflection crosstalk by the optical components.

An intraoral scanner includes an elongate wand that is used to scan inside a mouth (e.g., scan dental arches) of a patient. The intraoral scanner includes a probe housing disposed at a distal end of the elongate wand. At least a portion of the probe housing is to be inserted into a mouth of a patient for scanning. The probe housing forms an interior volume. Optical components (e.g., cameras, projectors, distributed projector, etc.) are disposed in the interior volume of the probe housing.

In some embodiments, the intraoral scanner includes a distributed projector disposed in the interior volume. In some embodiments, the distributed projector includes a diode module and a lens module that are located at least a threshold distance (e.g., remote) from each other (e.g., one or more cameras and/or projectors are disposed between the diode module and the lens module). The lens module may be located in a distal end of the probe housing (e.g., to illuminate the rearmost teeth of a dental arch between two cameras. The diode module may be located in a different portion of the intraoral scanner so that cameras are located between the lens module and the diode module.

The diode module includes a laser diode configured to emit a beam of light. In some embodiments, the diode module further includes focusing optics configured to focus the beam of light (e.g., cause the minimal spot size to be at a threshold distance from the intraoral scanner).

The lens module includes a pattern generating optical element configured to generate, based on at least a first portion of the beam of light, structured light to illuminate at least a portion of a mouth of a patient. In some embodiments, the lens module further includes a folding prism configured to deflect the at least a first portion of the beam of light received from the diode module to the pattern generating optical element. In some embodiments, the lens module further includes a relay lens configured to focus (e.g., re-focus) the beam of light).

The devices systems, and methods of the present disclosure have advantages over conventional systems. The intraoral scanner of the present disclosure has cameras and projectors that are smaller than those of conventional scanners. The intraoral scanner of the present disclosure may be less wide and less tall than conventional scanners. This allows the intraoral scanner of the present disclosure to have more efficient and quicker scanner maneuvering than conventional scanners. The intraoral scanner of the present disclosure may more easily reach certain portions of a mouth (e.g., rearmost molars, etc.) where conventional devices may not reach. The cameras and projectors of the present disclosure may drift less than those of conventional solutions. This allows the present disclosure to have more accurate image capture and modeling than conventional solutions. The intraoral scanner of the present disclosure may have less back reflection crosstalk than conventional solutions. This causes the intraoral scanner to have more accurate image capture and modeling than conventional solutions. The intraoral scanner of the present disclosure may not have or may have less components (e.g., heaters) that block image capture or reduce quality of image capture compared to conventional solutions. The intraoral scanner of the present disclosure has a decreased width compared to conventional scanners. This allows the intraoral scanner of the present disclosure to have more efficient and quicker scanner maneuvering than conventional scanners. The intraoral scanner of the present disclosure may more easily reach certain portions of a mouth (e.g., rearmost molars, etc.) where conventional devices may not reach. The intraoral scanner of the present disclosure can estimate a 3D surface with a higher degree of accuracy than conventional systems. This results in less time and processing and more accurately designed dental devices compared to conventional systems. The intraoral scanner of the present disclosure can estimate a 3D surface with a higher degree of accuracy than conventional systems. This results in less time and processing and more accurately designed dental devices compared to conventional systems.

Various embodiments are described herein. These various embodiments may be implemented as stand-alone solutions and/or may be combined. Accordingly, references to an embodiment, one embodiment, or some embodiments may refer to the same embodiment and/or to different embodiments. Some embodiments are discussed herein with reference to intraoral scans and intraoral images. However, embodiments described with reference to intraoral scans also apply to lab scans or model/impression scans. A lab scan or model/impression scan may include one or more images of a dental site or of a model or impression of a dental site, which may or may not include height maps, and which may or may not include intraoral two-dimensional (2D) images (e.g., 2D color images).

In some embodiments, the present disclosure describes intraoral scanners including projectors and cameras. In some embodiments, the projectors and/or cameras of the present disclosure may be part of a system that is not an intraoral scanner.

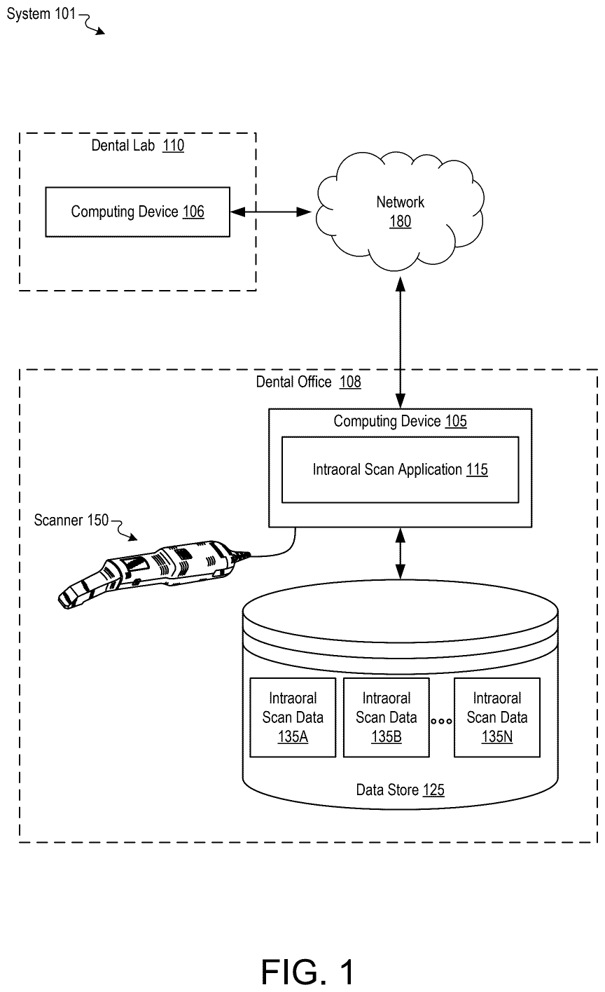

FIG. 1 illustrates a system 101 for performing intraoral scanning and/or generating a 3D surface and/or a virtual 3D model of a dental site, according to certain embodiments. System 101 includes a scanner 150. The scanner 150 may be the intraoral scanner of the present disclosure (e.g., intraoral scanner having ultra-low tip profile, intraoral scanner having one or more distributed projectors, etc.).

System 101 includes a dental office 108 and optionally one or more dental labs 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to one or more intraoral scanner 150 (also referred to as a scanner) and/or a data store 125 via a wired or wireless connection. In some embodiments, multiple scanners 150 in dental office 108 wirelessly connect to computing device 105. In some embodiments, scanner 150 is wirelessly connected to computing device 105 via a direct wireless connection. In some embodiments, scanner 150 is wirelessly connected to computing device 105 via a wireless network. In some embodiments, the wireless network is a Wi-Fi network. In some embodiments, the wireless network is a Bluetooth network, a Zigbee network, or some other wireless network. In some embodiments, the wireless network is a wireless mesh network, examples of which include a Wi-Fi mesh network, a Zigbee mesh network, and so on. In an example, computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

Computing device 106 may also be connected to a data store (not shown). The data stores may be local data stores and/or remote data stores. Computing device 105 and computing device 106 may each include one or more processing devices, memory, secondary storage, one or more input devices (e.g., such as a keyboard, mouse, tablet, touchscreen, microphone, camera, and so on), one or more output devices (e.g., a display, printer, touchscreen, speakers, etc.), and/or other hardware components.

In some embodiments, scanner 150 includes an inertial measurement unit (IMU). The IMU may include an accelerometer, a gyroscope, a magnetometer, a pressure sensor, and/or other type of sensor. For example, scanner 150 may include one or more micro-electromechanical system (MEMS) IMU. The IMU may generate inertial measurement data (also referred to as movement data), including acceleration data, rotation data, and so on.

Computing device 105 and/or data store 125 may be located at dental office 108 (as shown), at dental lab 110, or at one or more other locations such as a server farm that provides a cloud computing service. Computing device 105 and/or data store 125 may connect to components that are at a same or a different location from computing device 105 (e.g., components at a second location that is remote from the dental office 108, such as a server farm that provides a cloud computing service). For example, computing device 105 may be connected to a remote server, where some operations of intraoral scan application 115 are performed on computing device 105 and some operations of intraoral scan application 115 are performed on the remote server.

Some additional computing devices may be physically connected to the computing device 105 via a wired connection. Some additional computing devices may be wirelessly connected to computing device 105 via a wireless connection, which may be a direct wireless connection or a wireless connection via a wireless network. In embodiments, one or more additional computing devices may be mobile computing devices such as laptops, notebook computers, tablet computers, mobile phones, portable game consoles, and so on. In embodiments, one or more additional computing devices may be traditionally stationary computing devices, such as desktop computers, set top boxes, game consoles, and so on. The additional computing devices may act as thin clients to the computing device 105. In some embodiments, the additional computing devices access computing device 105 using remote desktop protocol (RDP). In some embodiments, the additional computing devices access computing device 105 using virtual network control (VNC). Some additional computing devices may be passive clients that do not have control over computing device 105 and that receive a visualization of a user interface of intraoral scan application 115. In some embodiments, one or more additional computing devices may operate in a master mode and computing device 105 may operate in a slave mode.

Intraoral scanner 150 may include a probe (e.g., a handheld probe) for optically capturing 3D structures. The intraoral scanner 150 may be used to perform an intraoral scan of a patient's oral cavity. An intraoral scan application 115 running on computing device 105 may communicate with the scanner 150 to effectuate the intraoral scan. A result of the intraoral scan may be intraoral scan data 135A, 135B through 135N that may include one or more sets of intraoral scans and/or sets of intraoral 2D images. Each intraoral scan may include a 3D image or point cloud that may include depth information (e.g., a height map) of a portion of a dental site. In embodiments, intraoral scans include x, y, and z information.

Intraoral scan data 135A-N may also include color 2D images and/or images of wavelengths (e.g., near-infrared (NIRI) images, infrared images, ultraviolet images, etc.) of a dental site in embodiments. In embodiments, intraoral scanner 150 alternates between generation of 3D intraoral scans and one or more types of 2D intraoral images (e.g., color images, NIRI images, etc.) during scanning. For example, one or more 2D color images may be generated between generation of a fourth and fifth intraoral scan by outputting white light and capturing reflections of the white light using multiple cameras.

Intraoral scanner 150 may include multiple different cameras (e.g., each of which may include one or more image sensors) that generate 2D images (e.g., 2D color images) of different regions of a patient's dental arch concurrently. These 2D images may be stitched together to form a single 2D image representation of a larger field of view that includes a combination of the fields of view of the multiple cameras. Intraoral 2D images may include 2D color images, 2D infrared or near-infrared (NIRI) images, and/or 2D images generated under other specific lighting conditions (e.g., 2D ultraviolet images). The 2D images may be used by a user of the intraoral scanner to determine where the scanning face of the intraoral scanner is directed and/or to determine other information about a dental site being scanned.

The scanner 150 may transmit the intraoral scan data 135A, 135B through 135N to the computing device 105. Computing device 105 may store the intraoral scan data 135A-135N in data store 125.

According to an example, a user (e.g., a practitioner) may subject a patient to intraoral scanning. In doing so, the user may apply scanner 150 to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 2D intraoral images (e.g., color 2D images) and/or 3D intraoral scans of particular teeth and/or regions of a dental site. In some embodiments, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the scanner 150 to the computing device 105 in the form of one or more points (e.g., one or more pixels and/or groups of pixels). For instance, the scanner 150 may provide an intraoral scan as one or more point clouds. The intraoral scans may each include height information (e.g., a height map that indicates a depth for each pixel).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture, or installation of a dental prosthesis at a dental site within the oral cavity (dental site), or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture, or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances.

In embodiments, intraoral scanning may be performed on a patient's oral cavity during a visitation of dental office 108. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches and may be performed to gather information for performing dental diagnostics, to generate a treatment plan, to determine progress of a treatment plan, and/or for other purposes. The dental information (intraoral scan data 135A-N) generated from the intraoral scanning may include 3D scan data, 2D color images, NIRI and/or infrared images, and/or ultraviolet images, of all or a portion of the upper jaw and/or lower jaw. The intraoral scan data 135A-N may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

For many prosthodontic procedures (e.g., to create a crown, bridge, veneer, etc.), an existing tooth of a patient is ground down to a stump. The ground tooth is referred to herein as a preparation tooth, or simply a preparation. The preparation tooth has a margin line (also referred to as a finish line), which is a border between a natural (unground) portion of the preparation tooth and the prepared (ground) portion of the preparation tooth. The preparation tooth is typically created so that a crown or other prosthesis can be mounted or seated on the preparation tooth. In many instances, the margin line of the preparation tooth is sub-gingival (below the gum line).

Intraoral scanners may work by moving the scanner 150 inside a patient's mouth to capture all viewpoints of one or more tooth. During scanning, the scanner 150 is calculating distances to solid surfaces in some embodiments. These distances may be recorded as images called ‘height maps’ or as point clouds in some embodiments. Each scan (e.g., optionally height map or point cloud) is overlapped algorithmically, or ‘stitched,’ with the previous set of scans to generate a growing 3D surface. As such, each scan is associated with a rotation in space, or a projection, to how it fits into the 3D surface.

During intraoral scanning, intraoral scan application 115 may register and stitch together two or more intraoral scans generated thus far from the intraoral scan session to generate a growing 3D surface. In some embodiments, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. A view of the 3D surface(s) may be periodically or continuously updated according to one or more viewing modes of the intraoral scan application. In one viewing mode, the 3D surface may be continuously updated such that an orientation of the 3D surface that is displayed aligns with a field of view of the intraoral scanner (e.g., so that a portion of the 3D surface that is based on a most recently generated intraoral scan is approximately centered on the display or on a window of the display) and a user sees what the intraoral scanner sees. In one viewing mode, a position and orientation of the 3D surface is static, and an image of the intraoral scanner is optionally shown to move relative to the stationary 3D surface.

Intraoral scan application 115 may generate one or more 3D surfaces from intraoral scans and may display the 3D surfaces to a user (e.g., a doctor) via a graphical user interface (GUI) during intraoral scanning. In embodiments, separate 3D surfaces are generated for the upper jaw and the lower jaw. This process may be performed in real time or near-real time to provide an updated view of the captured 3D surfaces during the intraoral scanning process. As scans are received, these scans may be registered and stitched to a 3D surface. Quality scores may be determined for various regions of the 3D surface based on one or more criteria as discussed in detail below. The quality scores may be continuously or periodically updated as information is added from further intraoral scans. As the quality scores gradually change, a visualization of the regions may change in accordance with the changes in the quality scores, enabling a user to have real time or near real time feedback on surface quality during scanning. Additionally, or alternatively, as scans are received the scanning process may be monitored to determine if a user is having trouble scanning any regions of a dental site (e.g., of the upper or lower dental arch). If a determination is made that a user is having trouble scanning a region of the dental site, then one or more remedial actions may be performed and/or one or more suggestions may be provided. Additionally, or alternatively, as scanning is being performed a zoom setting for displaying the 3D surface(s) may be dynamically determined based on one or more criteria, such as a velocity of the scanner and/or of a point of focus of the scanner. In embodiments, a user may select to enable or disable automatic zoom and/or automatic suggestions via the GUI. For example, the user may input a request for scanning assistance, which may cause automatic zoom and/or scanning suggestions to be enabled. These and other operations may be performed during scanning to improve a quality of the 3D surface(s), to speed up scanning, to help a user in trouble areas, and so on.

When a scan session or a portion of a scan session associated with a particular scanning role (e.g., upper jaw role, lower jaw role, bite role, etc.) is complete (e.g., all scans for an dental site or dental site have been captured), intraoral scan application 115 may generate a virtual 3D model of one or more scanned dental sites (e.g., of an upper jaw and a lower jaw). The final 3D model may be a set of 3D points and their connections with each other (i.e., a mesh). To generate the virtual 3D model, intraoral scan application 115 may register and stitch together the intraoral scans generated from the intraoral scan session that are associated with a particular scanning role. The registration performed at this stage may be more accurate than the registration performed during the capturing of the intraoral scans and may take more time to complete than the registration performed during the capturing of the intraoral scans. In some embodiments, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In some embodiments, registration is performed for adjacent or overlapping intraoral scans (e.g., each successive frame of an intraoral video). Registration algorithms are carried out to register two adjacent or overlapping intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

Intraoral scan application 115 may generate one or more 3D models from intraoral scans and may display the 3D models to a user (e.g., a doctor) via a graphical user interface (GUI). The 3D models can then be checked visually by the doctor. The doctor can virtually manipulate the 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction.

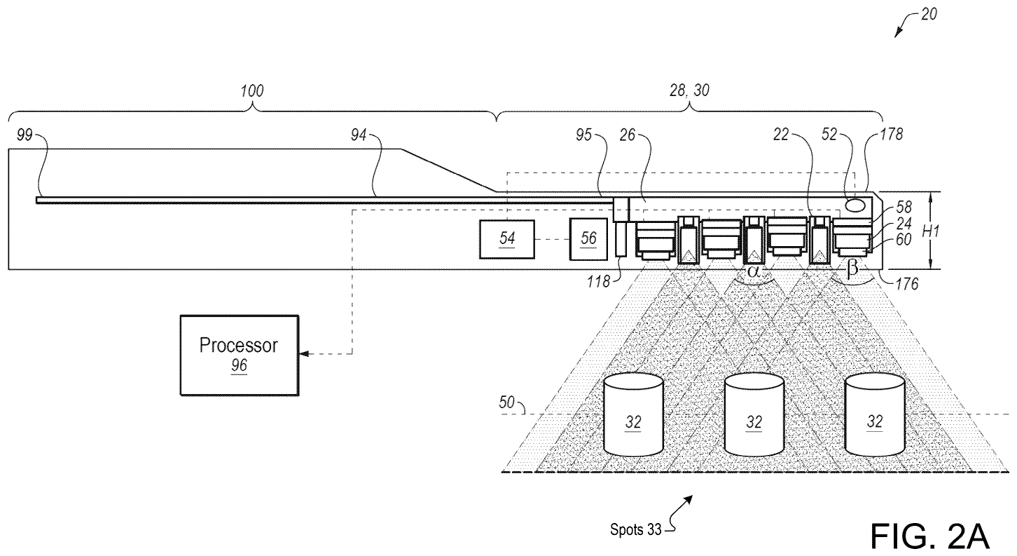

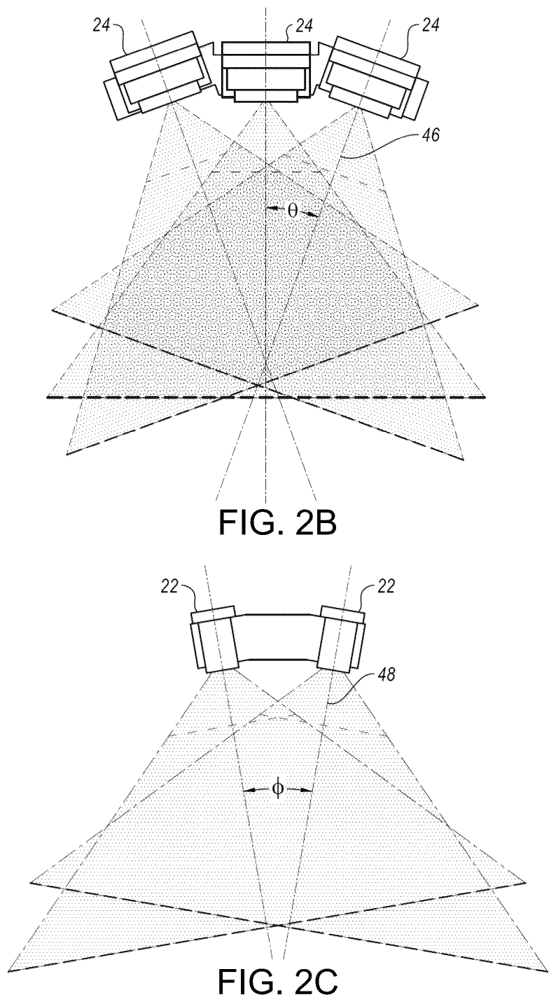

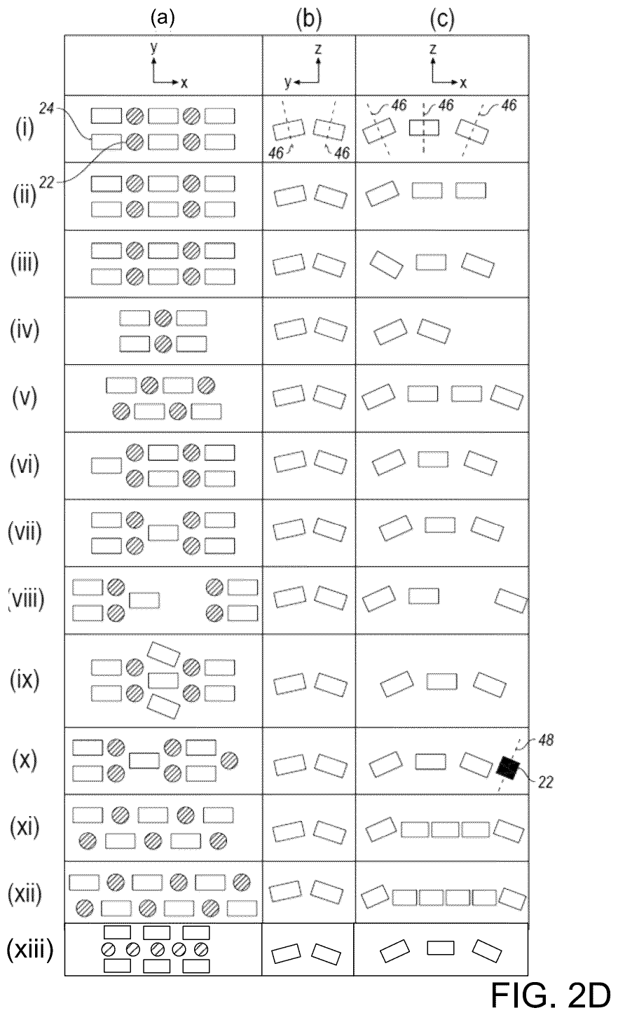

Reference is now made to FIG. 2A, which is a schematic illustration of an intraoral scanner 20 including an elongate handheld wand, according to certain embodiments. The intraoral scanner 20 may correspond to intraoral scanner 150 of FIG. 1 in some embodiments. Intraoral scanner 20 may be the intraoral scanner of the present disclosure (e.g., intraoral scanner having ultra-low tip profile, intraoral scanner having one or more distributed projectors, etc.).

Intraoral scanner 20 includes a plurality of structured light projectors 22 (e.g., projectors) and a plurality of cameras 24 that are coupled to a rigid structure 26 disposed within a probe 28 at a distal end 30 of the intraoral scanner 20. In some applications, during an intraoral scanning procedure, probe 28 is inserted into the oral cavity of a subject or patient.

For some applications, structured light projectors 22 are positioned within probe 28 such that each structured light projector 22 faces an object 32 outside of intraoral scanner 20 that is placed in its field of illumination, as opposed to positioning the structured light projectors in a proximal end of the handheld wand and illuminating the object by reflection of light off a mirror and subsequently onto the object. Alternatively, the structured light projectors may be disposed at a proximal end of the handheld wand. Similarly, for some applications, cameras 24 are positioned within probe 28 such that each camera 24 faces an object 32 outside of intraoral scanner 20 that is placed in its field of view, as opposed to positioning the cameras in a proximal end of the intraoral scanner and viewing the object by reflection of light off a mirror and into the camera. This positioning of the projectors and the cameras within probe 28 enables the scanner to have an overall large field of view while maintaining a low-profile probe. Alternatively, the cameras may be disposed in a proximal end of the handheld wand.

In some applications, cameras 24 each have a large field of view β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In some embodiments, a field of view β (beta) for each camera is between 80 and 90 degrees, which may be particularly useful because it provided a good balance among pixel size, field of view and camera overlap, optical quality, and cost. Cameras 24 may include an image sensor 58 and objective optics 60 including one or more lenses. To enable close focus imaging, cameras 24 may focus on an object focal plane 50 that is located between 1 mm and 30 mm, e.g., between 4 mm and 24 mm, e.g., between 5 mm and 11 mm, e.g., 9 mm-10 mm, from the lens that is farthest from the sensor. In some applications, cameras 24 may capture images at a frame rate of at least 30 frames per second, e.g., at a frame of at least 75 frames per second, e.g., at least 100 frames per second. In some applications, the frame rate may be less than 200 frames per second.

A large field of view achieved by combining the respective fields of view of all the cameras may improve accuracy due to reduced amount of image stitching errors, especially in edentulous regions, where the gum surface is smooth and there may be fewer clear high resolution 3D features. Having a larger field of view enables large smooth features, such as the overall curve of the tooth, to appear in each image frame, which improves the accuracy of stitching respective surfaces obtained from multiple such image frames.

Similarly, structured light projectors 22 may each have a large field of illumination a (alpha) of at least 45 degrees, e.g., at least 70 degrees. In some applications, field of illumination a (alpha) may be less than 120 degrees, e.g., than 100 degrees.

For some applications, to improve image capture, each camera 24 has a plurality of discrete preset focus positions, in each focus position the camera focusing on a respective object focal plane 50. Each of cameras 24 may include an autofocus actuator that selects a focus position from the discrete preset focus positions to improve a given image capture. Additionally or alternatively, each camera 24 includes an optical aperture phase mask that extends a depth of focus of the camera, such that images formed by each camera are maintained focused over all object distances located between 1 mm and 30 mm, e.g., between 4 mm and 24 mm, e.g., between 5 mm and 11 mm, e.g., 9 mm-10 mm, from the lens that is farthest from the sensor.

In some applications, structured light projectors 22 and cameras 24 are coupled to rigid structure 26 in a closely packed and/or alternating fashion, such that (a) a substantial part of each camera's field of view overlaps the field of view of neighboring cameras, and (b) a substantial part of each camera's field of view overlaps the field of illumination of neighboring projectors. Optionally, at least 20%, e.g., at least 50%, e.g., at least 75% of the projected pattern of light are in the field of view of at least one of the cameras at an object focal plane 50 that is located at least 4 mm from the lens that is farthest from the sensor. Due to different possible configurations of the projectors and cameras, some of the projected pattern may never be seen in the field of view of any of the cameras, and some of the projected pattern may be blocked from view by object 32 as the scanner is moved around during a scan.

Rigid structure 26 may be a non-flexible structure to which structured light projectors 22 and cameras 24 are coupled so as to provide structural stability to the optics within probe 28. Coupling all the projectors and all the cameras to a common rigid structure helps maintain geometric integrity of the optics of each structured light projector 22 and each camera 24 under varying ambient conditions, e.g., under mechanical stress as may be induced by the subject's mouth. Additionally, rigid structure 26 helps maintain stable structural integrity and positioning of structured light projectors 22 and cameras 24 with respect to each other.

Reference is now made to FIGS. 2B-2C, which include schematic illustrations of a positioning configuration for cameras 24 and structured light projectors 22 respectively, according to certain embodiments. The cameras 24 and/or structured light projectors 22 may be of the intraoral scanner of the present disclosure (e.g., intraoral scanner having ultra-low tip profile, intraoral scanner having one or more distributed projectors, etc.).

For some applications, to improve the overall field of view and field of illumination of the intraoral scanner 20, cameras 24 and structured light projectors 22 are positioned such that they do not all face the same direction. For some applications, such as is shown in FIG. 2B, a plurality of cameras 24 are coupled to rigid structure 26 such that an angle θ (theta) between two respective optical axes 46 of at least two cameras 24 is 90 degrees or less, e.g., 35 degrees or less. Similarly, for some applications, such as is shown in FIG. 2C, a plurality of structured light projectors 22 are coupled to rigid structure 26 such that an angle q (phi) between two respective optical axes 48 of at least two structured light projectors 22 is 90 degrees or less, e.g., 35 degrees or less.

Reference is now made to FIG. 2D, which is a chart depicting a plurality of different configurations for the position of structured light projectors 22 and cameras 24 in probe 28, according to certain embodiments. The cameras 24 and/or structured light projectors 22 may be of the intraoral scanner of the present disclosure (e.g., intraoral scanner having ultra-low tip profile, intraoral scanner having one or more distributed projectors, etc.).

Structured light projectors 22 are represented in FIG. 2D by circles and cameras 24 are represented in FIG. 2D by rectangles. It is noted that rectangles are used to represent the cameras, since typically, each image sensor 58 and the field of view β (beta) of each camera 24 have aspect ratios of 1:2. Column (a) of FIG. 2D shows a bird's eye view of the various configurations of structured light projectors 22 and cameras 24. The x-axis as labeled in the first row of column (a) corresponds to a central longitudinal axis of probe 28. Column (b) shows a side view of cameras 24 from the various configurations as viewed from a line of sight that is coaxial with the central longitudinal axis of probe 28 and substantially parallel to a viewing axis of the intraoral scanner. Similar to as shown in FIG. 2B, column (b) of FIG. 2D shows cameras 24 positioned so as to have optical axes 46 at an angle of 90 degrees or less, e.g., 35 degrees or less, with respect to each other. Column (c) shows a side view of cameras 24 of the various configurations as viewed from a line of sight that is perpendicular to the central longitudinal axis of probe 28.

Typically, the distal-most (toward the positive x-direction in FIG. 2D) and proximal-most (toward the negative x-direction in FIG. 2D) cameras 24 are positioned such that their optical axes 46 are slightly turned inwards, e.g., at an angle of 90 degrees or less, e.g., 35 degrees or less, with respect to the next closest camera 24. The camera(s) 24 that are more centrally positioned, i.e., not the distal-most camera 24 nor proximal-most camera 24, are positioned so as to face directly out of the probe, their optical axes 46 being substantially perpendicular to the central longitudinal axis of probe 28. It is noted that in row (xi) a projector 22 is positioned in the distal-most position of probe 28, and as such the optical axis 48 of that projector 22 points inwards, allowing a larger number of spots 33 projected from that particular projector 22 to be seen by more cameras 24.

In embodiments, the number of structured light projectors 22 in probe 28 may range from two, e.g., as shown in row (iv) of FIG. 2D, to six, e.g., as shown in row (xii). Typically, the number of cameras 24 in probe 28 may range from four, e.g., as shown in rows (iv) and (v), to seven, e.g., as shown in row (ix). It is noted that the various configurations shown in FIG. 2D are by way of example and not limitation, and that the scope of the present disclosure includes additional configurations not shown. For example, the scope of the present disclosure includes fewer or more than five projectors 22 positioned in probe 28 and fewer or more than seven cameras positioned in probe 28.

In an example application, an apparatus for intraoral scanning (e.g., an intraoral scanner 150) includes an elongate handheld wand including a probe at a distal end of the elongate handheld wand, at least two light projectors disposed within the probe, and at least four cameras disposed within the probe. Each light projector may include at least one light source configured to generate light when activated, and a pattern generating optical element that is configured to generate a pattern of light when the light is transmitted through the pattern generating optical element. Each of the at least four cameras may include a camera sensor (also referred to as an image sensor) and one or more lenses, wherein each of the at least four cameras is configured to capture a plurality of images that depict at least a portion of the projected pattern of light on an intraoral surface. A majority of the at least two light projectors and the at least four cameras may be arranged in at least two rows that are each approximately parallel to a longitudinal axis of the probe, the at least two rows including at least a first row and a second row.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may be positioned such that optical axes of the cameras in the first row are at an angle of 90 degrees or less with respect to optical axes of the cameras in the second row from a line of sight that is coaxial with the longitudinal axis of the probe. A remainder of the at least four cameras other than the distal-most camera and the proximal-most camera have optical axes that are substantially parallel to the longitudinal axis of the probe. Each of the at least two rows may include an alternating sequence of light projectors and cameras.

In a further application, the at least four cameras include at least five cameras, the at least two light projectors include at least five light projectors, a proximal-most component in the first row is a light projector, and a proximal-most component in the second row is a camera.

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row may be positioned such that the optical axes of the cameras in the first row are at an angle of 35 degrees or less with respect to the optical axes of the cameras in the second row from the line of sight that is coaxial with the longitudinal axis of the probe.

In a further application, the at least four cameras may have a combined field of view of 25-45 mm along the longitudinal axis and a field of view of 20-40 mm along a z-axis corresponding to distance from the probe.

Returning to FIG. 2A, for some applications, there is at least one uniform light projector 118 (which may be an unstructured light projector that projects light across a range of wavelengths) coupled to rigid structure 26. Uniform light projector 118 may transmit white light onto object 32 being scanned. At least one camera, e.g., one of cameras 24, captures 2D color images of object 32 using illumination from uniform light projector 118.

Processor 96 may run a surface reconstruction algorithm that may use detected patterns (e.g., dot patterns) projected onto object 32 to generate a 3D surface of the object 32. In some embodiments, the processor 96 may combine at least one 3D scan captured using illumination from structured light projectors 22 with a plurality of intraoral 2D images captured using illumination from uniform light projector 118 in order to generate a digital 3D image of the intraoral 3D surface. Using a combination of structured light and uniform illumination enhances the overall capture of the intraoral scanner and may help reduce the number of options that processor 96 needs to consider when running a correspondence algorithm used to detect depth values for object 32. In some embodiments, the intraoral scanner and correspondence algorithm described in U.S. application Ser. No. 16/446,181, filed Jun. 19, 2019, is used. U.S. application Ser. No. 16/446,181, filed Jun. 19, 2019, is incorporated by reference herein in its entirety. In embodiments, processor 96 may be a processor of computing device 105 of FIG. 1. Alternatively, processor 96 may be a processor integrated into the intraoral scanner 20.

For some applications, all data points taken at a specific time are used as a rigid point cloud, and multiple such point clouds are captured at a frame rate of over 10 captures per second. The plurality of point clouds is then stitched together using a registration algorithm, e.g., iterative closest point (ICP), to create a dense point cloud. A surface reconstruction algorithm may then be used to generate a representation of the surface of object 32.

For some applications, at least one temperature sensor 52 is coupled to rigid structure 26 and measures a temperature of rigid structure 26. Temperature control circuitry 54 disposed within intraoral scanner 20 (a) receives data from temperature sensor 52 indicative of the temperature of rigid structure 26 and (b) activates a temperature control unit 56 in response to the received data. Temperature control unit 56, e.g., a PID controller, keeps probe 28 at a desired temperature (e.g., between 35 and 43 degrees Celsius, between 37 and 41 degrees Celsius, etc.). Keeping probe 28 above 35 degrees Celsius, e.g., above 37 degrees Celsius, reduces fogging of the glass surface of intraoral scanner 20, through which structured light projectors 22 project and cameras 24 view, as probe 28 enters the intraoral cavity, which is typically around or above 37 degrees Celsius. Keeping probe 28 below 43 degrees, e.g., below 41 degrees Celsius, prevents discomfort or pain.

In some embodiments, heat may be drawn out of the probe 28 via a heat conducting element 94, e.g., a heat pipe, that is disposed within intraoral scanner 20, such that a distal end 95 of heat conducting element 94 is in contact with rigid structure 26 and a proximal end 99 is in contact with a proximal end 100 of intraoral scanner 20. Heat is thereby transferred from rigid structure 26 to proximal end 100 of intraoral scanner 20. Alternatively, or additionally, a fan disposed in a handle region 174 of intraoral scanner 20 may be used to draw heat out of probe 28.

FIGS. 2A-2D illustrate one type of intraoral scanner that can be used for embodiments of the present disclosure. However, embodiments are not limited to the illustrated type of intraoral scanner. In some embodiments, intraoral scanner 150 corresponds to the intraoral scanner described in U.S. application Ser. No. 16/910,042, filed Jun. 23, 2020 and entitled “Intraoral 3D Scanner Employing Multiple Miniature Cameras and Multiple Miniature Pattern Projectors”, which is incorporated by reference herein. In some embodiments, intraoral scanner 150 corresponds to the intraoral scanner described in U.S. application Ser. No. 16/446,181, filed Jun. 19, 2019 and entitled “Intraoral 3D Scanner Employing Multiple Miniature Cameras and Multiple Miniature Pattern Projectors”, which is incorporated by reference herein.

In some embodiments an intraoral scanner that performs confocal focusing to determine depth information may be used. Such an intraoral scanner may include a light source and/or illumination module that emits light (e.g., a focused light beam or array of focused light beams). The light passes through a polarizer and through a unidirectional mirror or beam splitter (e.g., a polarizing beam splitter) that passes the light. The light may pass through a pattern before or after the beam splitter to cause the light to become patterned light. Along an optical path of the light after the unidirectional mirror or beam splitter are optics, which may include one or more lens groups. Any of the lens groups may include only a single lens or multiple lenses. One of the lens groups may include at least one moving lens.

The light may pass through an endoscopic probing member, which may include a rigid, light-transmitting medium, which may be a hollow object defining within it a light transmission path or an object made of a light transmitting material, e.g., a glass body or tube. In some embodiments, the endoscopic probing member includes a prism such as a folding prism. At its end, the endoscopic probing member may include a mirror of the kind ensuring a total internal reflection. Thus, the mirror may direct the array of light beams towards a teeth segment or other object. The endoscope probing member thus emits light, which optionally passes through one or more windows and then impinges on to surfaces of intraoral objects.

The light may include an array of light beams arranged in an X-Y plane, in a Cartesian frame, propagating along a Z axis, which corresponds to an imaging axis or viewing axis of the intraoral scanner. Responsive to the surface on which the incident light beams hits being an uneven surface, illuminated spots may be displaced from one another along the Z axis, at different (Xᵢ, Yᵢ) locations. Thus, while a spot at one location may be in focus of the confocal focusing optics, spots at other locations may be out-of-focus. Therefore, the light intensity of returned light beams of the focused spots will be at its peak, while the light intensity at other spots will be off peak. Thus, for each illuminated spot, multiple measurements of light intensity are made at different positions along the Z-axis. For each of such (Xᵢ, Yᵢ) location, the derivative of the intensity over distance (Z) may be made, with the Zᵢ yielding maximum derivative, Z₀, being the in-focus distance.

The light reflects off intraoral objects and passes back through windows (if they are present), reflects off of the mirror, passes through the optical system, and is reflected by the beam splitter onto a detector. The detector is an image sensor having a matrix of sensing elements each representing a pixel of the scan or image. In some embodiments, the detector is a charge coupled device (CCD) sensor. In some embodiments, the detector is a complementary metal-oxide semiconductor (CMOS) type image sensor. Other types of image sensors may also be used for detector. In some embodiments, the detector detects light intensity at each pixel, which may be used to compute height or depth.

Alternatively, in some embodiments an intraoral scanner that uses stereo imaging is used to determine depth information.

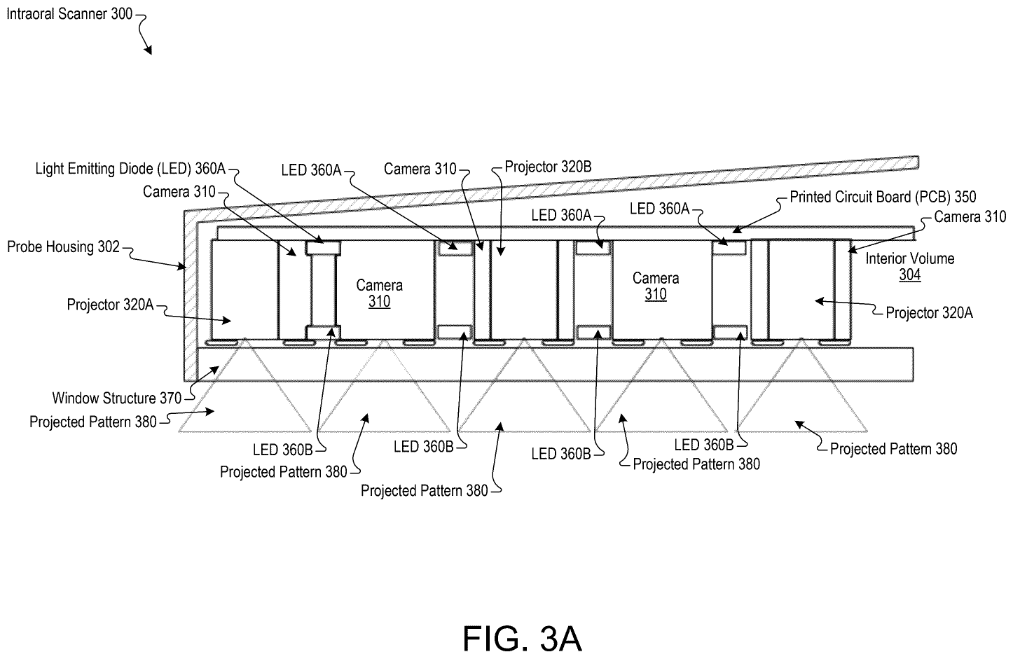

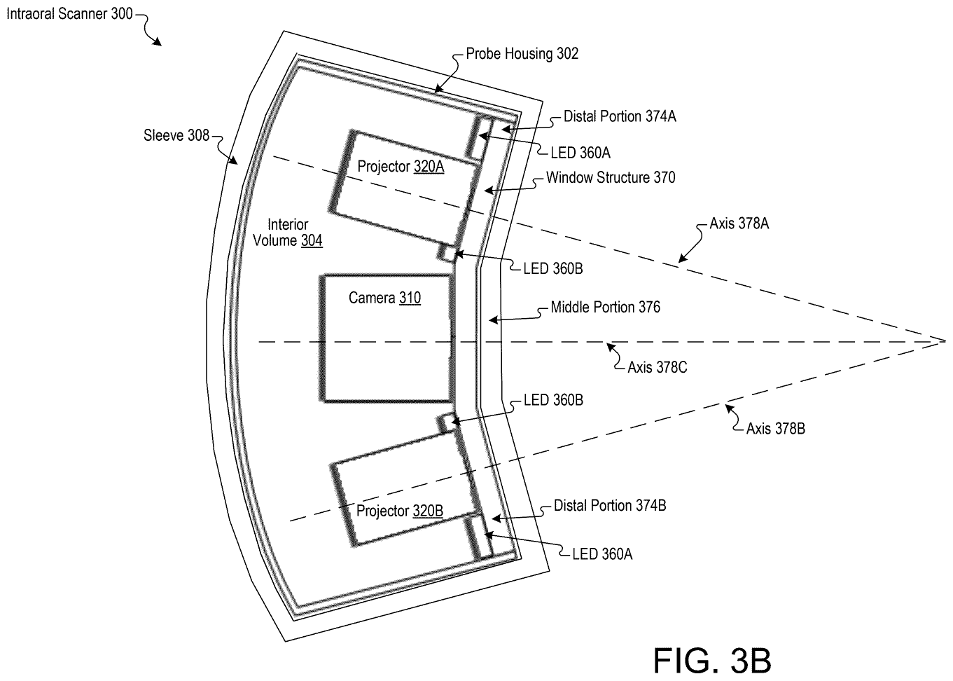

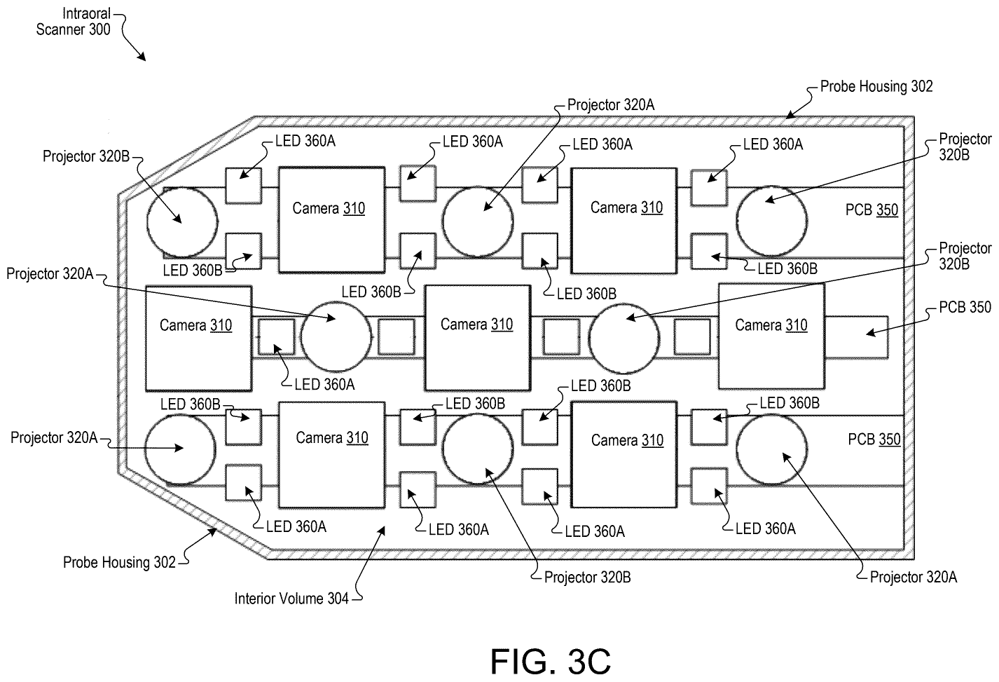

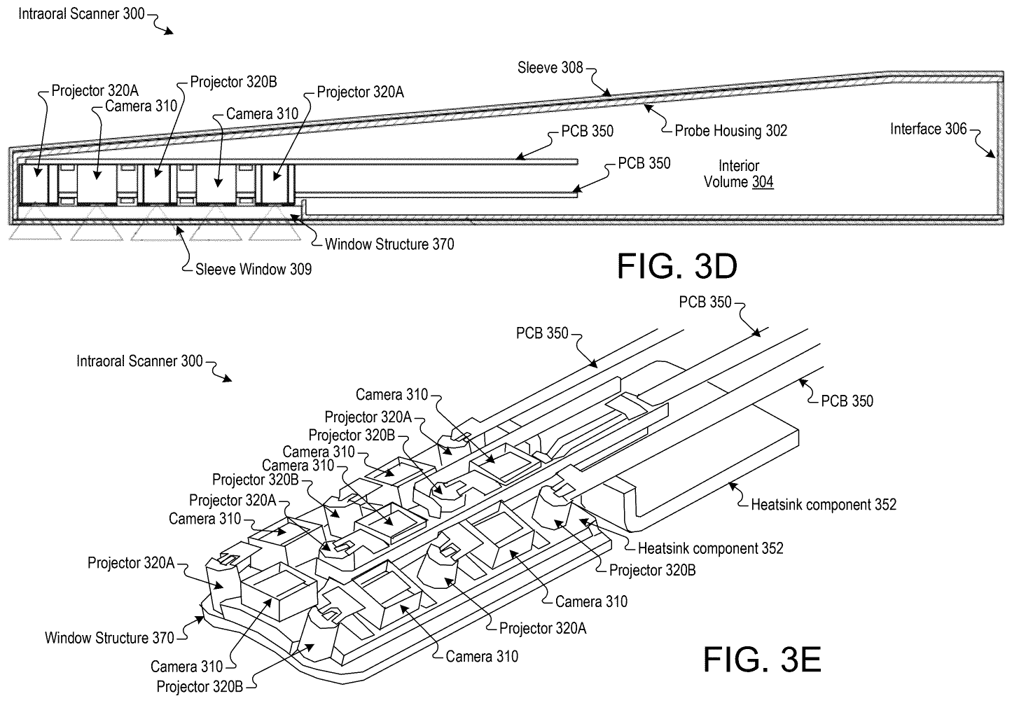

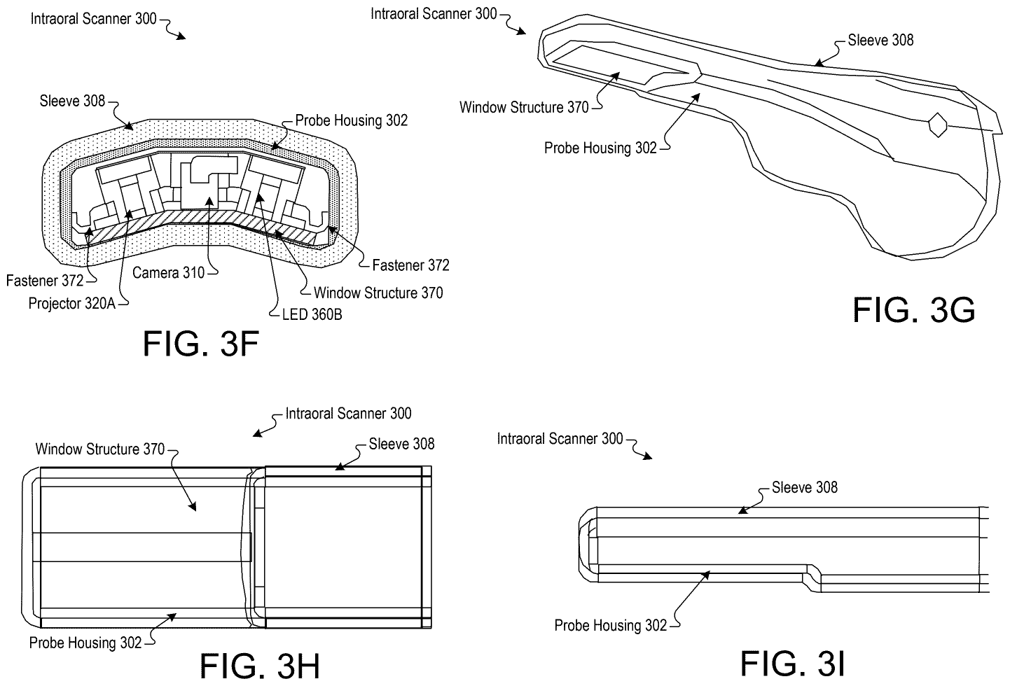

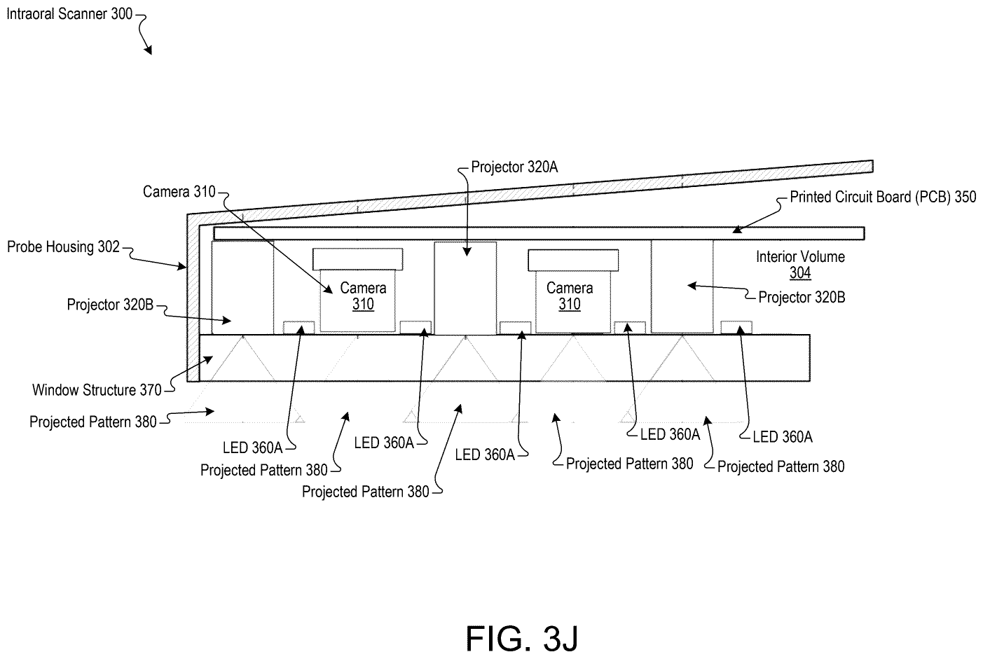

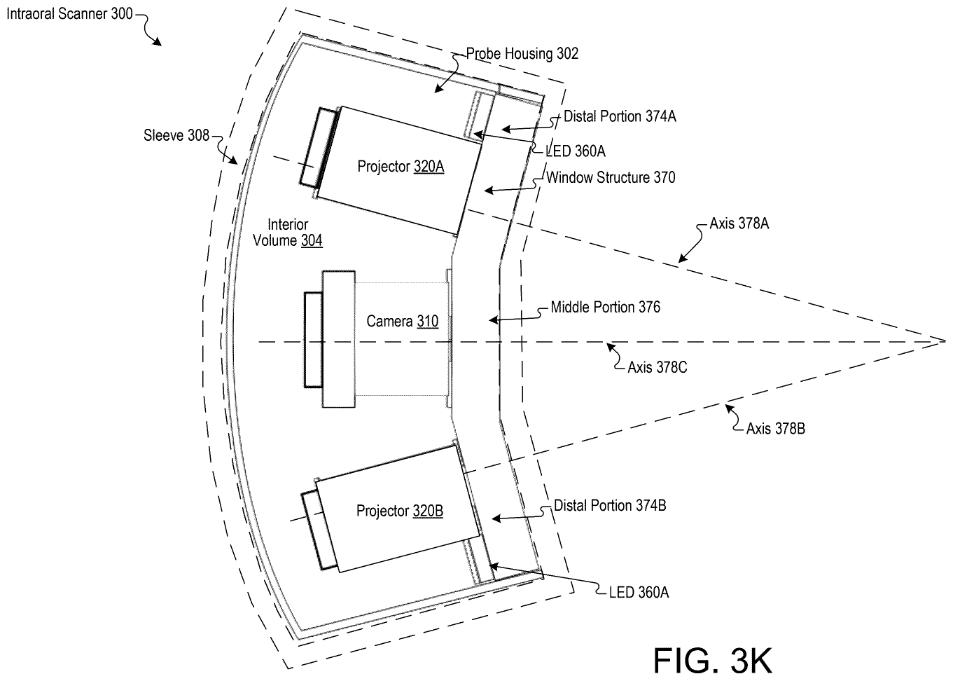

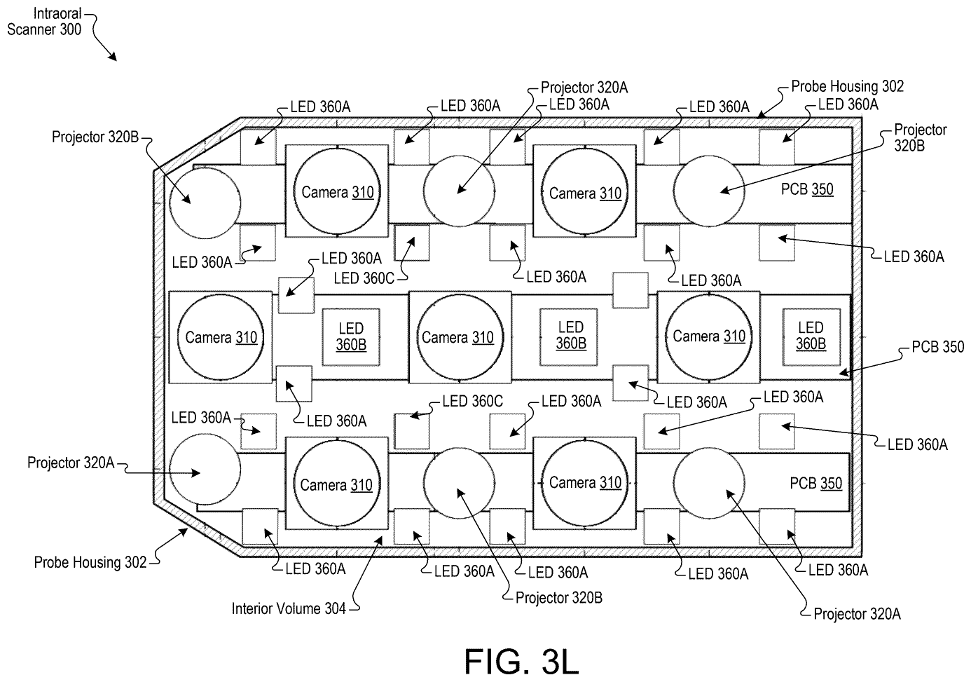

FIGS. 3A-L illustrate components of intraoral scanners 300 (e.g., that have a slim tip), according to certain embodiments. In some embodiments, intraoral scanners 300 of one or more of FIGS. 3A-L include similar or the same functionality, components, materials, and/or the like as scanner 150 of FIG. 1 and/or intraoral scanner 20 of FIGS. 2A-D.

In some embodiments, intraoral scanner 300 of one or more of FIGS. 3A-L includes one or more of the features of FIGS. 1-7.

In some embodiments, the intraoral scanner of one or more of FIGS. 3A-L includes: a probe housing disposed at a distal end of an elongate wand, the probe housing forming an interior volume; and a distributed projector disposed in the interior volume, the distributed projector comprising: a diode module comprising a laser diode configured to emit a beam of light; and a lens module comprising a first pattern generating optical element configured to generate structured light based on at least a first portion of the beam of light, the lens module being disposed at least a threshold distance from the diode module. In some embodiments, the first pattern generating optical element is at least one of a multi lens array (MLA) or a diffractive optical element (DOE). In some embodiments, the lens module further comprises a first folding prism configured to deflect the at least a first portion of the beam of light to the first pattern generating optical element. In some embodiments, the lens module further comprises a second folding prism and a second pattern generating optical element; the first folding prism is configured to transmit a second portion of the beam of light to the second folding prism; and the second folding prism is configured to deflect the second portion of the beam of light to the second pattern generating optical element. In some embodiments, the lens module further comprises a relay lens configured to focus the beam of light. In some embodiments, the relay lens is disposed between the diode module and a folding prism of the lens module. In some embodiments, the relay lens is disposed between a first folding prism of the lens module and the first pattern generating optical element. In some embodiments, the relay lens is further configured to deflect at least the first portion of the beam of light to the first pattern generating optical element. In some embodiments, the diode module further comprises focusing optics configured to focus the beam of light, the focusing optics being disposed between the laser diode and the lens module. In some embodiments, the first pattern generating optical element is disposed on a window structure of the intraoral scanner. In some embodiments, the intraoral scanner further comprises a plurality of cameras disposed in a camera portion of the intraoral scanner, wherein the camera portion of the intraoral scanner is disposed between the diode module and the lens module. In some embodiments, the beam of light emitted from the diode module is to pass between two or more cameras and is to be received by the lens module. In some embodiments, the intraoral scanner further comprises a first camera and a second camera, the first camera and the second camera being disposed in a distal portion of the intraoral scanner, wherein the lens module is disposed between the first camera and the second camera. In some embodiments, the intraoral scanner further comprises: a window coupled to the probe housing, wherein the lens module is disposed at or proximate to a first distal end of the window and the diode module is disposed at or proximate to a second distal end of the window. In some embodiments, the intraoral scanner further comprises a window coupled to the probe housing, wherein the window covers the lens module and does not cover the diode module. In some embodiments, the lens module is separated from the diode module by air, an optical fiber, or a light guide. In some embodiments,

In some embodiments, the intraoral scanner of one or more of FIGS. 3A-L includes: a probe housing disposed at a distal end of an elongate wand, the probe housing forming an interior volume; and a distributed projector disposed in the interior volume, the distributed projector comprising: a diode module configured to emit a first beam of light at a first wavelength; and a first prism component comprising: a first beam splitter configured to deflect a first portion of the first beam of light and transmit a second portion of the first beam of light; and a second beam splitter configured to deflect the second portion of the first beam of light. In some embodiments, the intraoral scanner further comprises: a lens module disposed at least a threshold distance from the diode module, the lens module comprising: a relay lens configured to focus the first portion of the first beam of light; a folding prism configured to deflect the first portion of the first beam of light; and a pattern generating optical element configured to generate first structured light based on the first portion of the first beam of light. In some embodiments, the diode module is further configured to emit a second beam of light at a second wavelength. In some embodiments, the intraoral scanner further comprises: a second prism component comprising: a third beam splitter configured to deflect a first portion of the second beam of light and transmit a second portion of the second beam of light; and a fourth beam splitter configured to deflect the second portion of the second beam of light. In some embodiments, the first beam splitter and the second beam splitter are further configured to transmit the second beam of light, wherein the first prism component further comprises: a third beam splitter configured to deflect a first portion of the second beam of light and transmit a second portion of the second beam of light; and a fourth beam splitter configured to deflect the second portion of the second beam of light. In some embodiments, the distributed projector is of an intraoral scanner, the distributed projector includes: a diode module comprising a laser diode configured to emit a beam of light; and a lens module disposed at least a threshold distance from the diode module, the lens module comprising: a folding prism configured to deflect at least a first portion of the beam of light; and a pattern generating optical element configured to generate, based on the at least a first portion of the beam of light, structured light to illuminate at least a portion of a mouth of a patient. In some embodiments, the diode module further comprises focusing optics configured to focus the beam of light. In some embodiments, the lens module further comprises a relay lens configured to focus the beam of light. In some embodiments,

In some embodiments, the intraoral scanner of one or more of FIGS. 3A-L includes: a probe housing disposed at a distal end of an elongate wand, the probe housing forming an interior volume; a non-distributed projector disposed in the interior volume; and a distributed projector disposed in the interior volume, the distributed projector comprising a diode module and a lens module. In some embodiments: the non-distributed projector comprising components configured to emit a first beam of light and generate first structured light; the components are less than a threshold distance from each other; the diode module is configured to emit a second beam of light; the lens module is configured to generate second structured light based on at least a portion of the second beam of light; and the lens module is disposed greater than the threshold distance from the diode module. In some embodiments, the lens module is disposed at a distal end of the probe housing. In some embodiments, the distal end of the probe housing has an angled tip that houses the lens module.

In some embodiments, the intraoral scanner of one or more of FIGS. 3A-L is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view of about 70 to about 90 degrees; and one or more non-object-facing imaging devices disposed within the interior volume in a rear portion of the probe housing, wherein the one or more non-object-facing imaging devices are configured to capture respective images via respective imaging axes that reflect to pass through the window structure, and wherein the one or more object-facing imaging devices have a second field of view of about 10 to about 30 degrees. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in the rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors. In some embodiments, the hybrid intraoral scanner further includes a fold mirror disposed at a distal end of the probe housing, wherein the fold mirror is configured to reflect the respective imaging axes and the respective projector axes to provide structured light 3D capture of a distal portion of a user mouth, and wherein the one or more non-object-facing imaging devices are oriented substantially parallel to the window structure. In some embodiments, the hybrid intraoral scanner further includes a prism disposed at a distal end of the probe housing, wherein the prism is configured to reflect the respective imaging axes and the respective projector axes to provide structured light 3D capture of a distal portion of a user mouth, wherein the prism has a fold angle of about 25 degrees to about 40 degrees, and wherein the one or more non-object-facing imaging devices are oriented substantially parallel to the window structure. In some embodiments, the hybrid intraoral scanner further includes a dichroic filter, wherein the one or more non-object-facing imaging devices comprise one or more upward-facing cameras that are oriented away from the window structure and an optical coherence tomography (OCT) channel that is oriented substantially parallel to the window structure, wherein the one or more upward-facing cameras are oriented upward and are configured to combine coaxially with a forward pointing OCT channel via the dichroic filter. In some embodiments, a first camera of the one or more object-facing imaging devices is configured to provide a viewfinder associated with moving the hybrid intraoral scanner. In some embodiments, In some embodiments, the one or more object-facing imaging devices and the one or more non-object-facing imaging devices are configured to provide imaging of one or more of a three-dimensional (3D) surface, subgingival, occlusal caries, proximal caries, or periodontal pockets. In some embodiments, the one or more object-facing imaging devices and the one or more non-object-facing imaging devices are configured to provide structured near-infrared (NIRI) images to provide shallow 3D viewing of hard tissue lesions. In some embodiments, the one or more object-facing imaging devices and the one or more object-facing projectors are configured to perform surface 3D capture. In some embodiments, the hybrid intraoral scanner further comprises an array of object-facing LEDs associated with two-dimensional color capture. In some embodiments, the one or more non-object-facing projectors comprise one or more of a rear NIRI structured light or NIRI collimated projector for subsurface 3D capture. In some embodiments, the one or more non-object-facing imaging devices comprise an OCT imaging channel coaxial configured for surface and sub-surface 3D structure capture. In some embodiments, the one or more non-object-facing imaging devices comprises an endoscopic OCT device inserted between the one or more object-facing imaging devices and the one or more object-facing projectors, wherein the endoscopic OCT device comprises a built-in rotating fold prism configured to provide transverse line scanning. In some embodiments, image data from the one or more non-object-facing imaging devices and the one or more object-facing imaging devices is to be combined to provide a third field of view that is greater than at least one of the first field of view or the second field of view. In some embodiments, the hybrid intraoral scanner further comprises a collimator, a scan mirror, and a fold mirror, wherein the one or more non-object-facing imaging devices comprise an OCT measuring arm fiber configured to provide light that is to pass through the collimator, reflect off the scan mirror, and reflect off the fold mirror to pass through the window structure.

In some embodiments, the intraoral scanner of one or more of FIGS. 3A-L is used with a method that includes: identifying first image data from one or more object-facing imaging devices at a first field of view of about 70 to about 90 degrees, the one or more object-facing imaging devices being disposed within an interior volume formed by a probe housing and a window structure of a hybrid intraoral scanner, the one or more object-facing imaging devices being disposed proximate the window structure; identifying second image data from one or more non-object-facing imaging devices at a second field of view of about 10 to about 30 degrees, the one or more non-object-facing imaging devices being disposed within the interior volume in a rear portion of the probe housing; and combining the first image data and the second image data. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in a rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors.

In some embodiments, the intraoral scanner of one or more of FIGS. 3A-L is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view; and an optical coherence tomography (OCT) imaging channel disposed within the interior volume in a rear portion of the probe housing, wherein the OCT imaging channel is configured to capture respective images via a respective imaging axis that reflects to pass through the window structure, and wherein the OCT imaging channel has a second field of view that is different from the first field of view. In some embodiments, the hybrid intraoral scanner further includes one or more object-facing projectors disposed within the interior volume proximate the window structure, wherein the one or more object-facing projectors provide corresponding light via corresponding projector axes that directly pass through the window structure. In some embodiments, the one or more object-facing devices and the one or more object-facing projectors are closer to a distal tip of the probe housing than the OCT imaging channel.

In some embodiments, the intraoral scanner of one or more of FIGS. 3A-L includes: a processing device configured to: receive a first image of an object from a first camera of the intraoral scanner, the first camera having a first static focal distance; determine a more sharp portion of the first image and a less sharp portion of the first image, the less sharp portion corresponding to a region of the object; for the less sharp portion of the first image, determine a more sharp portion of a second image corresponding to the region of the object, the second image being from a second camera of the intraoral scanner, the second camera having a second static focal distance that is different from the first static focal distance of the first camera; generate a combined image using the more sharp portion of the first image and the more sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface. In some embodiments, the processing device is further configured to receive a third image of the object from a third camera of the intraoral scanner; the third camera has a third static focal distance that is different from the first static focal distance of the first camera and the second static focal distance of the second camera; and the processing device is to generate the combined image further based on the third image. In some embodiments: the first camera is proximate a distal end of a tip of the intraoral scanner; the third camera is further from the distal end of a tip of the intraoral scanner than the first camera; the second camera is disposed between the first camera and the third camera; the first static focal distance of the first camera is less than the second static focal distance of the second camera; and the second static focal distance of the second camera is less than the third static focal distance of the third camera. In some embodiments, the combined image has an increased sharpness than the sharp portion of the first image and the sharp portion of the second image. In some embodiments, the first image of the object being captured by the first camera and the second image of the object being captured by the second camera in a similar location, in a similar angle, and at a similar brightness level. In some embodiments, the first camera and second camera capture images of the mouth of the user responsive to a light source of the intraoral scanner providing white light or near infrared light into the mouth of the user. In some embodiments, to generate the combined image, the processing device is to smooth the more sharp portion of the first image and the more sharp portion of the second image.

In some embodiments, the intraoral scanner of one or more of FIGS. 3A-L includes: a processing device configured to: receive a first image of an object from a first camera of the intraoral scanner, the first camera having a first static focal distance; receive a second image of the object from a second camera of the intraoral scanner, the second camera having a second static focal distance that is different from the first static focal distance; determine a first sharp portion of the first image corresponding to a first region of the object; determine a second sharp portion of the second image corresponding to the first region of the object; generate a combined image using the first sharp portion of the first image and the second sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface.

FIG. 3A illustrates a side cross-sectional view of an intraoral scanner 300. FIG. 3B illustrates a front cross-sectional view of an intraoral scanner 300 (e.g., front view transverse cross section). FIG. 3C illustrates a bottom cross-sectional view of an intraoral scanner 300 (e.g., bottom view optical section). FIG. 3D illustrates a side cross-sectional view of an intraoral scanner 300 (e.g., side view longitudinal cross section). FIG. 3E illustrates a top perspective view of components of an intraoral scanner 300. FIG. 3F is a front cross-sectional view of an intraoral scanner 300. FIG. 3G illustrates a bottom perspective view of an intraoral scanner 300. FIG. 3H illustrates a bottom view of an intraoral scanner 300. FIG. 3I illustrates a side view of an intraoral scanner 300. FIG. 3J illustrates a side cross-sectional view of an intraoral scanner 300. FIG. 3K illustrates a front cross-sectional view of an intraoral scanner 300 (e.g., front view transverse cross section). FIG. 3L illustrates a bottom cross-sectional view of an intraoral scanner 300 (e.g., bottom view optical section). FIGS. 3A-C may illustrate a first layout of an intraoral scanner 300 and FIGS. 3J-L may illustrate a second layout of an intraoral scanner 300.

The intraoral scanner 300 may be a wand that is connected to a computation station. The tip of the intraoral scanner 300 may be inserted into the oral cavity of a person while scanning procedure is performed. A sleeve may be placed over the tip of the intraoral scanner 300 prior to being inserted in the oral cavity. The sleeve may be disposable and/or replaceable in some embodiments. In some embodiments, the sleeve is reusable. For example, the sleeve may be autoclavable in embodiments. The sleeve is a protective sleeve that protects the intraoral scanner 300 from contact with bodily fluids of a patient.

The tip of the intraoral scanner 300 may be part of an assembly that is separate from the rest of the wand. Once the assembly of the tip of the intraoral scanner 300 is completed, the tip can be added to the wand. The tip may connect to the wand via mechanical interfaces (e.g., screws, springs, bolts, fasteners) and electrical connections (e.g., connect to internal portion of scanner). The tip of the intraoral scanner 300 may be a standalone item that is later integrated with the rest of the intraoral scanner 300 (e.g., endpiece, scanner). The intraoral scanner 300 may use multi-structured light to create a 3D model. In some embodiments, the intraoral scanner 300 has cameras 310 (e.g., disposed in square holes formed by the intraoral scanner 300). The intraoral scanner 300 may have projectors 320A-B that are disposed adjacent to and/or between the cameras 310. In some embodiments, the projectors 320B have a first type of illumination (e.g., blue illuminations) and the projectors 320B have a second type of illumination (e.g., green illumination). The multiple cameras 310 and multiple projectors 320 may be used to provide sufficient data for 3D construction (e.g., 3D imaging) of the dental site.

In some embodiments, intraoral scanner 300 includes a probe housing 302 (e.g., thin stainless steel enclosure) disposed at a distal end of an elongate wand. The probe housing 302 may form an interior volume 304. The intraoral scanner 300 may include optical components disposed in the interior volume 304. The optical components may include cameras 310 and projectors 320A-B. The optical components (e.g., cameras 310 and projectors 320A-B) may be orthogonal to a corresponding portion of the window structure 370. The cameras 310 may have imaging axes that are orthogonal to a corresponding portion of the window structure 370. The projectors 320A-B may have projection axes that are orthogonal to a corresponding portion of the window structure 370.

The window structure 370 may include a distal portion 374A, a middle portion 376, and a distal portion 374B. The distal portion 374A may be disposed at a first orientation and the distal portion 374B may be disposed at a second orientation that is at an angle to the first orientation. The middle portion 376 may be disposed between the distal portion 374A and the distal portion 374B. The middle portion 376 may have a third orientation that is different from the first orientation and the second orientation.

The optical components may have different axes. Each of one or more of the optical components proximate the distal portion 374A of the window structure 370 may have a corresponding axis 378A that is orthogonal to the distal portion 374A. Each of one or more of the optical components proximate the distal portion 374B of the window structure 370 may have a corresponding axis 378B that is orthogonal to the distal portion 374B. Each of one or more of the optical components proximate the middle portion 376 of the window structure 370 may have a corresponding axis 378C that is orthogonal to the middle portion 376. A projector 320 disposed proximate the distal portion 374A and a projector 320 disposed proximate the distal portion 374B may illuminate two sides of a dental site. A camera 310 disposed proximate the distal portion 374A and a camera 310 disposed proximate the distal portion 374B may capture images on two sides of a dental site (e.g., perform teeth wrapping imaging).

A sleeve 308 may be disposed around the probe housing 302 and window structure 370. In some embodiments, the sleeve 308 has a concave-shaped window that mirrors the shape of the concave-shaped window structure 370. In some embodiments, the sleeve 308 is transparent and has a portion that aligns with the window structure 370 and has a concave shape that mirrors the shape of the concave-shaped window structure 370.

In some embodiments, the probe housing 320 has a concave-shaped (e.g., bow-shape tip) transversal cross-section (e.g., see FIG. 3B, concave transverse cross section). An upper surface (e.g., upper wall) of the probe housing 302 may be curved (e.g., concave upper wall). A first camera 310 may be disposed at a first angle (e.g., in the bow-shaped tip transversal cross-section) and a second camera 310 may be disposed at a second angle (e.g., in the bow shape tip transversal cross-section). A first central axis of the first camera 310 may intersect with a second central axis of the second camera 310 below the probe housing. The first camera 310 and the second camera 310 may capture images to perform teeth wrapping imaging. Teeth wrapping imaging may including capturing images that overlap each other from different angles to form a 3D mold of the dental arch.

In some embodiments, projectors 320 include ultraminiature pattern projectors. In some examples, the optical components (e.g., a first row of optical components) may include a first ultraminiature pattern projector, a second ultraminiature pattern projector, and a camera 310 disposed between the first ultraminiature pattern projector and the second ultraminiature pattern projector. In some examples, the optical components (e.g., a second row of optical components) may include a first camera 310, a second camera 310, and an ultraminiature pattern projector disposed between the first camera 310 and the second camera 310. In some embodiments, at least a portion of the optical components (e.g., the first row of optical components including the camera 310, the first ultraminiature pattern projector, and the second ultraminiature pattern projector) are disposed within 5 millimeters of a foremost end of the probe housing 302.

In some embodiments, each projector 320 (e.g., ultraminiature pattern projector, first ultraminiature pattern projector and second ultraminiature pattern projector) has a diameter of less than 4 millimeters (e.g., about 2 millimeters) and a height of less than 4.5 millimeters (e.g., about 3 millimeters).

In some embodiments, the probe housing 302 has a height of less than 10 millimeters (e.g., about 7.5 to about 8.5 millimeters) and a width of less than 20 millimeters (about 16 to about 17 millimeters).

In some embodiments, the intraoral scanner 300 (e.g., concave-shaped transversal cross-section, the bow-shaped tip transversal cross-section, concave transverse cross section) is configured to cause about 20% to about 40% overlap of image capture via two or more cameras 310 (e.g., first camera 310 and second camera 310 in a row of optical components).

In some embodiments, the cameras 310 (e.g., first camera 310 and second camera 310) are to capture images. The images are to be used to perform model building via a correspondence algorithm or machine learning.

In some embodiments, the optical components (e.g., cameras 310) are configured to capture images of rearmost teeth in a mouth of a patient.

In some embodiments, the probe housing 302 forms an opening. The intraoral scanner may include a window structure 370 (e.g., structural window, optical window) coupled to the probe housing 302. The window structure 370 may cover the opening. The window structure 370 and the probe housing 302 may form the interior volume 304. The optical components (e.g., cameras 310 and projectors 320) may be disposed in the interior volume 304. The optical components (e.g., cameras 310 and projectors 320) may be bonded directly to the window structure 370 (via an adhesive, via an adhesive that is optically permeable, a high-level anti-contamination sealing adhesive). The optical components (e.g., cameras 310 and projectors 320) may be bonded directly to the window structure 370 to provide drift-free retention of the optical components.

In some embodiments, the window structure 370 (e.g., a segmented front glass optical window) includes a first distal portion disposed at a first angle and a second distal portion at a second angle. The window structure is configured to block back reflection crosstalk by the optical components (e.g., cameras 310, projectors 320). The optical components are secured to the window structure 370 to prevent back reflection of light emitted from the projectors 320 from being back reflected from the window structure 370 to the cameras 310 (e.g., light from projectors 320 passes through the window structure 370 instead of being reflected from the window structure 370 to the cameras 310). The window structure 370 (e.g., segmented front glass optical window) may be a single piece of glass formed via glass molding (e.g., precision glass molding, heating a single piece of glass and forming the heated single piece of glass). In some embodiments, the window structure 370 is heat treated to have portions at different angles (e.g., the window structure 370 was a single structure before being heat treated). In some embodiments, the window structure 370 is multiple components that are joined together via heat treatment to have portions at different angles.

In some embodiments, the probe housing 302 is wrapped by a heat sink metal structure that is in thermal contact with the probe housing 302.

In some embodiments, a heating element is disposed between two or more of the optical components (e.g., one or more cameras 310 and/or one or more projectors 320) to heat the intraoral scanner 300. The heating element may be configured to quickly (e.g., in less than a minute, in less than 30 seconds) heat the intraoral scanner to a working temperature (e.g., room temperature, above room temperature, etc.) to be used in a mouth of a patient.

In some embodiments, the intraoral scanner 300 includes a sleeve 308 that includes an optical window (e.g., a sleeve window structure that is coupled to a sleeve housing). The sleeve may be configured to be removably disposed over the probe housing 302. The optical window of the sleeve may be configured to substantially align with the window structure 370 that is coupled to the probe housing 302. The optical window of the sleeve 308 may include a first portion that approximately aligns with a distal portion 374A of the window structure 370, a second portion that approximately aligns with a distal portion 374B of the window structure 370, and a third portion that approximately aligns with a middle portion 376 of the window structure 370 (e.g., the middle portion 376 of the window structure 370 being disposed between the distal portion 374A and the distal portion 374B).

The window structure 370 may have a concave transverse cross section.

A first optical component may have a first axis that is orthogonal to the first distal portion of the window structure. A second optical component may have a second axis that is orthogonal to the second distal portion of the window structure. A third optical component may have a third axis that is orthogonal to a third portion of the window structure (e.g., the third portion of the window structure being disposed between the first distal portion and the second distal portion). In some embodiments, the first optical component includes a first structured light projector, the second optical component includes a camera, and the third optical component includes a second structured light projector. In some embodiments, the first optical component includes a first camera, the second optical component includes a structured light projector, and the third optical component includes a second camera.

The intraoral scanner 300 (e.g., probe housing 302 disposed at a distal end of an elongate wand) may have an ultra-low tip profile. The intraoral scanner may be configured to perform multi-structural triangulation 3D capture.

In some embodiments, the intraoral scanner 300 (e.g., probe housing 302 disposed at a distal end of an elongate wand) may be an elongated item that includes miniature cameras 310 and miniature projectors 320 for use in the intraoral scanner 300.

The intraoral scanner 300 may employ miniature object facing cameras 310 and miniature object facing projectors 320 to facilitate high-speed 3D intraoral scanning. The intraoral scanner 300 may employ one or more arrays of object facing LEDs 360 or other light projectors. Although LEDs may be described in some embodiments, other types of light producing components may be used in some embodiments. In some embodiments, the LEDs 360 include an array of object facing white LEDs 360A for 2D color capture, an array of object facing near-infrared imaging (NIRI) LEDs 360B for NIRI capture, and one or more blue LEDs 306C. NIRI may be an alternative to radiographs and may use nonionizing radiation in the near-infrared spectrum to differentially scatter light off tooth surfaces and generate images allowing interproximal detection (e.g., caries detection).

The intraoral scanner 300 may have a sealed enclosure (e.g., probe housing 302 sealed to window structure 370) to facilitate high level cross contamination avoidance.

The intraoral scanner 300 (e.g., probe housing 302 and window structure 370) may have a concave-shaped transversal cross section (e.g., bow shape tip transversal cross section, concave transverse cross section) supporting teeth wrapping imaging.

The intraoral scanner 300 may have optical components (e.g., cameras 310 and projectors 320) bonded directly (e.g., via glue, transparent bonding material, etc.) to window structure 370 (e.g., front optical glass window) for drift free retention.

The window structure 370 may include a first distal portion disposed at a first angle and a second distal portion at a second angle (e.g., a segmented front glass optical window) to block back reflection crosstalk (e.g., by geometry of the window structure 370).

The intraoral scanner 300 (e.g., probe housing 302 and window structure 370) may have a long yet slim cross section to allow efficient scanner maneuvering (e.g., relaxed and stress-free maneuvering) inside the oral cavity of a patient including access to hard to capture niches like last molar of the dental arch of the patient.

The intraoral scanner 300 (e.g., probe housing 302 and window structure 370) may be compatible with single piece disposable sleeve for cross contamination control.

The intraoral scanner 300 (e.g., probe housing 302 and window structure 370) may have a heating element (e.g., for temperature control, for de-fogging) that is not disposed between the optical components and the objects being imaged (e.g., heating element does not block the window structure 370, the heating element is not an indium tin oxide (ITO) defogging system disposed on the window structure 370). ITO may be a transparent conductor that can be used as a heating element over a window. Heating elements that are not ITO defogging system (e.g., non-ITO defogging element) are heating elements (e.g., non-transparent heating elements) that are not in the line of sight of the cameras 310 and projectors 320.

The intraoral scanner 300 (e.g., probe housing 302 and window structure 370) has a slim profile tip that has a window structure 370 that is a glass structural member. The intraoral scanner 300 (e.g., probe housing 302 and window structure 370) may be a concave shape (e.g., bow shape). The intraoral scanner 300 (e.g., probe housing 302 and window structure 370) may include miniature cameras 310 and miniature projectors 320. The intraoral scanner 300 (e.g., probe housing 302 and window structure 370) may be used for multi-structural triangulation.

The intraoral scanner 300 may have an elongated and slim front tip (e.g., probe housing 302 and window structure 370) that uses miniature object-facing cameras 310, pattern projectors 320, and LEDs 360 (e.g., white LEDs 360A, near-infrared (NIR) LEDs 360B, and one or more blue LEDs 306C. The optical components (e.g., cameras 310 and projectors 320) are directly bonded by their foremost surface onto window structure 370 (e.g., a structurally rigid optical glass serving as optical front window, sealing member, and structural element).

The intraoral scanner 300 may have optical components that are configured to perform tip-mounted structured triangulation (e.g., without a fold mirror). The optical components may be configured to be located proximate objects (e.g., teeth) that are to be scanned. The intraoral scanner 300 may have a low tip profile and may provide a large field of view (FOV) (e.g., angular extent of the observable world that is seen at any given moment).

The intraoral scanner 300 may have a reduced tip cross section compared to conventional scanners. This may provide increased maneuverability. The optical components (e.g., cameras 310, projectors 320) of the intraoral scanner 300 may be closer to the tip distal end than conventional scanners. This may allow imaging of rearmost molars. The intraoral scanner 300 may have tighter camera-projector overlap than conventional scanners. This may improve image capture quality and coverage. The intraoral scanner 300 may have projectors 320 and cameras 310 distributed to provide triangulation diversity and reduced occlusions.

The projector 320 may be an ultraminiature laser projector (ULP). The projector 320 may have a height of less than 4.2 mm and a width (e.g., diameter) of less than 4.5 mm. The projector 320 may have a height of about 3 mm and a width (e.g., diameter) of about 2 mm.

The camera 310 may be an ultraminiature camera module (UCM). The camera 310 may have a height of less than 4.5 mm, a width of less than 5.5 mm, and a depth of less than 5.5 mm. The camera 310 may be equal to or less than about 3×3×3 mm³ (equal to or less than about 27 mm³). The camera 310 may have a height of about 2.9 mm, a width of about 3 mm, and a depth of about 2.8 mm. The camera 310 may have micro-objective lens (e.g., miniature aspherical lenses). The camera 310 may have a field of view (FOV) of greater than 80 degrees.

The intraoral scanner 300 may have optical components directly bonded onto the window structure 370 (e.g., bow-shape segmented glass window). The intraoral scanner 300 may have a low profile (e.g., probe housing 302 has height of less than 10 mm or less than 8 mm or less than 7 mm), narrow width (e.g., probe housing 302 has width of less than 20 mm or less than 18 mm or less than 16 mm), negligible back reflections (e.g., optical components are adhered to the window structure 370), non-ITO defogging heating element, single piece all-clear polymer sleeve (e.g., sleeve 308), and/or denser configuration for better overlap (e.g., optical components are closer to each other than conventional scanners).

The intraoral scanner 300 may be inserted into a mouth of a patient until hitting a stop surface. The foremost capture aperture of the intraoral scanner 300 may reach deeper in a mouth of a patient than conventional scanners. The foremost capture aperture of the intraoral scanner 300 may reach less than 8 mm, less than 6 mm, or less than 4 mm from a stop surface in the mouth of a patient.

The intraoral scanner 300 may have a high maneuverability and high patient convenience. The probe housing 302 may have a height of less than about 15 mm, a height of less than about 14 mm, or a height of less than about 8 mm. The probe housing 302 may have a width of less than about 26 mm, a width of less than about 23 mm, or a width of less than about 17 mm. The intraoral scanner 300 may have a higher capture quality (e.g., correspondence) of number of spots captured by two cameras or more than conventional scanners. The intraoral scanner 300 may have a larger probability (e.g., greater than 30%, 40%, 50%, or 60%) of last molar capturability compared to conventional scanners.

In some embodiments, FIGS. 3A-C are different views of the same intraoral scanner 300. Probe housing 302 and window structure 370 form an interior volume 304. Cameras 310 and projectors 320 are adhered to the window structure 370 within the interior volume 304. Cameras, projectors 320, LEDs 360A, LEDs 360B, and/or LEDs 306C are electrically coupled to printed circuit boards (PCBs) 350. As shown in FIG. 3A, each projector 320 may provide a projected pattern 380. As shown in FIG. 3B, the window structure 370 has a planar middle portion that is surrounded by a first angled side portion and a second angled side portion.

A PCB 350 may include light emitting diodes (LEDs) 360. LEDs 360A may be white LEDs. LEDs 360B may be infrared LEDs. LEDs 360C may be blue LEDs. LEDs 360A may be used for color reconstruction.

In some embodiments, a PCB 350 is secured (e.g., soldered) to the projectors 320 subsequent to the projectors being positioned on (e.g., adhered to window structure 370). In some embodiments, the projectors 320 and cameras 310 are positioned in the window structure 370 with adhesive, the projectors 320 and cameras 310 are soldered to one or more PCBs 350, and then the adhesive between the window structure 370 and the projectors 320 and cameras 310 is cured in an oven (e.g., projectors 320 and cameras 310 are fixed into place in the window structure 370). Each projector 320 and camera 310 may be fixed to a designated location by using thermally conductive adhesive, which also improves the thermal connection of the projector 320 and camera 310 to the window structure 370.

In some embodiments, the projectors 320B are based on blue laser illumination and the projectors 320A are based on green laser illumination. Each projector 320 may have three legs, where two legs (e.g., anode and cathode) are soldered to a PCB 350 and the third leg is ground.

The PCB 350 may form holes that align with the corresponding orientation of each of the projectors 320 and/or cameras 310 (e.g., −10, −5, 0, 5, and 10 degrees). The soldering of the PCB 350 to the projectors 320 and/or cameras 310 maintains the projectors 320 and/or cameras 310 in the correct orientations until the adhesive is cured.

Two or more of the projectors 320 are at different orientations (e.g., angles, rotations) so that the projected patterns 380 are not parallel. This prevents ambiguity of which projected pattern 380 is from which projector 320. Two or more of the projectors 320 are angled relative to each other (e.g., to generate angle differentiation between projected patterns 380) to avoid ambiguity of projected patterns 380.

FIG. 3D illustrates an intraoral scanner 300 including probe housing (e.g., stainless steel enclosure), window structure 370 (e.g., tip segmented glass window), optical components (e.g., cameras 310, projectors 320) bonded to the window structure 370, PCBs 350 connecting optical components (e.g., cameras 310, projectors 320, LEDs 360, etc.) to other components (e.g., in the wand body, processing device, etc.), interface 306 (e.g., to wand body), sleeve 308 (e.g., single piece sleeve), and/or sleeve window 309 (e.g., sleeve optical window). In some embodiments, the sleeve 308 is a transparent material and sleeve window 309 is the same material as the rest of the sleeve 308 (e.g., sleeve window 309 and sleeve housing of sleeve 308 are the same component). In some embodiments, sleeve window 309 is transparent and is different from the sleeve housing of the sleeve 308 (e.g., sleeve window 309 and sleeve housing of sleeve 308 are different components). The interface 306 may couple the probe housing 302 to a wand body of the intraoral scanner 300.

Referring to FIG. 3E, intraoral scanner 300 may include optical components (e.g., projectors 320 and cameras 310) bonded (e.g., via adhesive) to the window structure 370. Intraoral scanner 300 may include one or more heatsink components 352 (e.g., aluminum heatsink components). In some embodiments, a heatsink component 352 is disposed in the interior volume 304 around one or more of the optical components (e.g., projectors 320 and cameras 310). A heatsink component 352 may be disposed proximate (e.g., above) the window structure 370. A heatsink component 352 may be disposed in the probe housing 302 without being above the window structure 370.

FIGS. 3F-I illustrate views of an intraoral scanner 300. FIG. 3F is a front cross-sectional view, FIG. 3G is a bottom perspective view, FIG. 3H is a bottom view, and FIG. 3I is a side view of intraoral scanner 300.

Referring to FIG. 3F, sides of window structure 370 may be secured to probe housing 302 (e.g., via adhesive) and an upper surface of window structure 370 may be secured to probe housing 302 (e.g., via fasteners 372).

In some embodiments, FIGS. 3J-L are different views of the same intraoral scanner 300. Probe housing 302 and window structure 370 form an interior volume 304. In some embodiments, cameras 310 and/or projectors 320 are adhered to the window structure 370 and/or a metal structure within the interior volume 304. Cameras, projectors 320, LEDs 360A, LEDs 360B, and/or LEDs 306C are electrically coupled to printed circuit boards (PCBs) 350. As shown in FIG. 3J, each projector 320 may provide a projected pattern 380. As shown in FIG. 3K, the window structure 370 has a planar middle portion that is surrounded by a first angled side portion and a second angled side portion. In some embodiments, in FIGS. 3J-L, there are no projectors 320 on the center row (compared to FIGS. 3A-C). In some embodiments, in FIGS. 3J-L, one or more of the cameras 310 (e.g., each camera 310) is neighbored by green and blue projectors in a symmetrical manner (e.g., except for the front triple of optical components due to functional constraints). In some embodiments, the optical components are glued to the metal structure (e.g., probe housing 302) that surrounds the optical components instead of being glued to the window structure 370.

U.S. patent application Ser. No. 18/226,651 to Atiya, et al., is assigned to the assignee of the present application, and is incorporated herein by reference, describes an ultraminiature pattern projector. One or more of projectors 320 may have the same or similar functionality, components, material, etc. as one or more of the ultraminiature pattern projectors described in U.S. patent application Ser. No. 18/226,651 to Atiya, et al. In some embodiments, each projector 320 (e.g., structured light projector) includes a housing, within which is disposed a light source. In some embodiments the housing is a sealed housing (e.g., is hermetically sealed). Each light source includes at least one semiconductor laser die and at least one beam shaping optical element. In some embodiments, the semiconductor laser die and the beam shaping optical element are disposed within a common chamber of the housing. Placing the beam shaping optical element and the semiconductor laser die of the structured light projector within the same chamber of the housing enables a distance between an emission point of the semiconductor laser die and an input face of the beam shaping optical element to be shorter than conventional laser diodes permit. Distance D between an emission point of the semiconductor laser diode and an input face of the beam shaping optical element is at least 50 microns and/or less than 250 microns. Some examples of the advantages provided are: overall reduction in size of the structured light projector, in turn enabling a reduction in size of the probe as well as increased flexibility in the arrangement of the structured light projectors and the cameras; increased collection efficiency of the laser light; increased depth of focus of the structured light projector; use of multiple laser dies within a single structured light projector, increasing the quantity of structured light features used for 3D reconstruction without increasing the size of and/or number of structured light projectors; and/or reduced speckle noise when using multiple laser dies.

Each projector 320 (e.g., structured light projector) may include at least one pattern generating optical element positioned such that each structured light projector projects a pattern of light onto an intraoral surface when the light source of the projector 320 is activated to emit light through the pattern generating optical element of the structured light projector.

An intraoral scanner 300 may include: an elongate handheld wand including a probe housing 302 at a distal end of the handheld wand; and one or more projectors 320 (e.g., structured light projectors) disposed within the probe housing 302, each projector 320 including a housing, a light source disposed within the housing, and a pattern generating optical element. The light source may include a semiconductor laser die and a beam shaping optical element. A distance D between an emission point of the semiconductor laser die and an input face of the beam shaping optical element is 50-250 microns. Each projector 320 is configured to project a pattern of light onto an intraoral surface when the light source of the structured light projector is activated to emit light through the pattern generating optical element of the structured light projector.

In some embodiments, the housing is a sealed housing. In some embodiments, the semiconductor laser die and the beam shaping optical element being disposed within the housing (e.g., within the sealed housing, within a common chamber of the housing) and distance D being 50-250 microns together allow a longest dimension of the sealed housing to be 1.5-2.5 mm.

In some embodiments, a height of the housing is 1.6-2.4 mm. In some embodiments, the beam shaping optical element is positioned within the housing such that at least 75% of the light emitted by the semiconductor laser die enters the beam shaping optical element. In some embodiments, the beam shaping optical element is positioned within the housing such that 80-90% of the light emitted by the semiconductor laser die enters the beam shaping optical element. In some embodiments, the housing includes metal and the semiconductor laser die is disposed within the housing such that heat is conducted from the semiconductor laser die to the metal of the housing. In some embodiments, the semiconductor laser die is mounted on a submount within the housing such that heat is conducted from the semiconductor laser die to the metal of the housing through the submount. In some embodiments, the submount is ceramic.

In some embodiments, the intraoral scanner 300 further includes one or more cameras 310 disposed within the probe housing 302, where a distance between (i) an optical axis of at least one camera 310 and (ii) an optical axis of at least one projector 320 (e.g., structured light projector) that is adjacent the at least one camera 310 is 3-5 mm.

U.S. patent application Ser. No. 17/869,698 to Atiya, et al., published as US20230025243A1 to Atiya, et. al, is assigned to the assignee of the present application, and is incorporated herein by reference, describes an intraoral scanner with illumination sequencing and controlled polarization. The intraoral scanner 300 may have the same or similar functionality, components, material, etc. as one or more of the embodiments described in U.S. patent application Ser. No. 18/226,651 to Atiya, et al. In some embodiments, a correspondence algorithm is used with the cameras 310 and the projectors 320 of intraoral scanner 300.

In some embodiments, each camera 310 includes a camera sensor that has an array of pixels, for each of which there exists a corresponding ray in 3-D space originating from the pixel whose direction is towards an object being imaged; each point along a particular one of these rays, when imaged on the sensor, will fall on its corresponding respective pixel on the sensor. The term used for this may be a “camera ray.” Similarly, for each projected spot from each projector 320 there exists a corresponding projector ray. Each projector ray corresponds to a respective path of pixels on at least one of the camera sensors, i.e., if a camera 310 sees a spot projected by a specific projector ray, that spot is detected by a pixel on the specific path of pixels that corresponds to that specific projector ray. Values for (a) the camera ray corresponding to each pixel on the camera sensor of each of the cameras, and (b) the projector ray corresponding to each of the projected spots of light from each of the projectors, may be stored during a calibration process.

In some embodiments, based on the stored calibration values a processing device may be used to run a correspondence algorithm in order to identify a 3D location for each projected spot on the surface. For a given projector ray, the processing device “looks” at the corresponding camera sensor path on one of the cameras 310. Each detected spot along that camera sensor path will have a camera ray that intersects the given projector ray. That intersection defines a 3D point in space. The processing device then searches among the camera sensor paths that correspond to that given projector ray on the other cameras 310 and identifies how many other cameras 310, on their respective camera sensor paths corresponding to the given projector ray, also detected a spot whose camera ray intersects with that 3D point in space. If two or more cameras 310 detect spots whose respective camera rays intersect a given projector ray at the same 3D point in space, the cameras 310 are considered to “agree” on the spot being located at that 3D point. Accordingly, the processing device may identify 3D locations of the projected light (e.g., projected) pattern of light) based on agreements of the two or more cameras 310 on there being the projected pattern of light by projector rays at certain intersections. The process is repeated for the additional spots along a camera sensor path, and the spot for which the highest number of cameras “agree” is identified as the spot that is being projected onto the surface from the given projector ray. A 3D position on the surface is thus computed for that spot.

In some embodiments, once a position on the surface is determined for a specific spot, the projector ray that projected that spot, as well as all camera rays corresponding to that spot, may be removed from consideration and the correspondence algorithm may be run again for a next projector ray. Ultimately, the identified 3D locations may be used to generate a digital 3D model of the intraoral surface.

U.S. patent application Ser. No. 17/869,698 to Atiya, et al., published as US20230025243A1 to Atiya, et. al, is assigned to the assignee of the present application, and is incorporated herein by reference, describes an intraoral scanner with illumination sequencing and controlled polarization. The intraoral scanner 300 may have the same or similar functionality, components, material, etc. as one or more of the embodiments described in U.S. patent application Ser. No. 18/226,651 to Atiya, et al. In some embodiments, a correspondence algorithm is used with the cameras 310 and the projectors 320 of intraoral scanner 300.

U.S. Patent Application No. 63/461,804 to Dafna, et al., is assigned to the assignee of the present application, and is incorporated herein by reference, describes determining 3D data for 2D points using machine learning. The intraoral scanner 300 may have the same or similar functionality, components, material, etc. as one or more of the embodiments described in U.S. Patent Application No. 63/461,804 to Dafna, et al. In some embodiments, machine learning is used with the cameras 310 and the projectors 320 of intraoral scanner 300 to determine 3D data (e.g., modeling of a dental arch) using 2D points.

In some embodiments, a method includes projecting, by one or more projectors 320 (e.g., structured light projectors) of an intraoral scanner 300, a light pattern including projector rays onto a dental site. The method may further include capturing, by cameras 310 of the intraoral scanner 300, images of at least a portion of the light pattern projected onto the dental site, where each camera 310 captures an image including points of at least the portion of the light pattern projected onto the dental site. The method may further include determining, for each projector ray, one or more candidate points that might have been caused by the projector ray.

In some embodiments, the method includes: processing information for each projector ray using a trained machine learning model, where the trained machine learning model generates one or more outputs including, for each projector ray, and for each candidate point associated with the projector ray, a probability that the candidate point corresponds to the projector ray; and determining 3D coordinates for at least some of the points in the images based on the one or more outputs of the trained machine learning model.

In some embodiments, the method includes: using a trained machine learning model to select candidate points for projector rays based on one or more inputs including probabilities of candidate points corresponding to projector rays; and determining 3D coordinates for at least some of the points in the images based on the selected candidate points for the plurality of projector rays.

In some embodiments, a method includes: using a first trained machine learning model to determine probabilities that captured points of a captured light pattern in one or more images correspond to projected points of a projected light pattern; using a second trained machine learning model to determine correspondence between a plurality of the captured points and the projected points based on one or more of the determined probabilities; and determining depth information for at least some of the plurality of captured points based on the determined correspondence.

In some embodiments, a method includes: using one or more trained machine learning models to determine correspondence between captured points of a captured light pattern in images and projected points of a projected light pattern; and determining depth information for at least some of the plurality of captured points based on the determined correspondence.

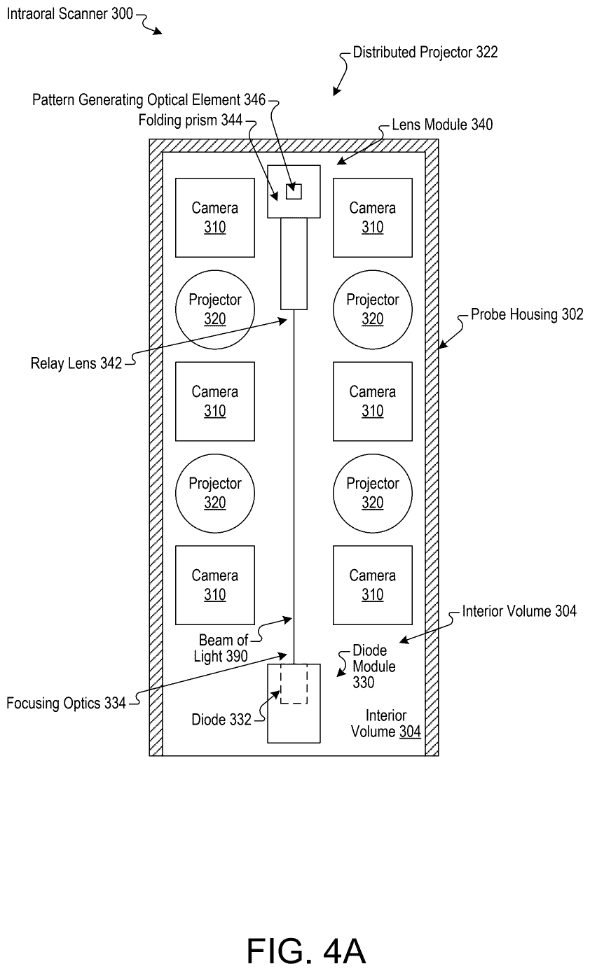

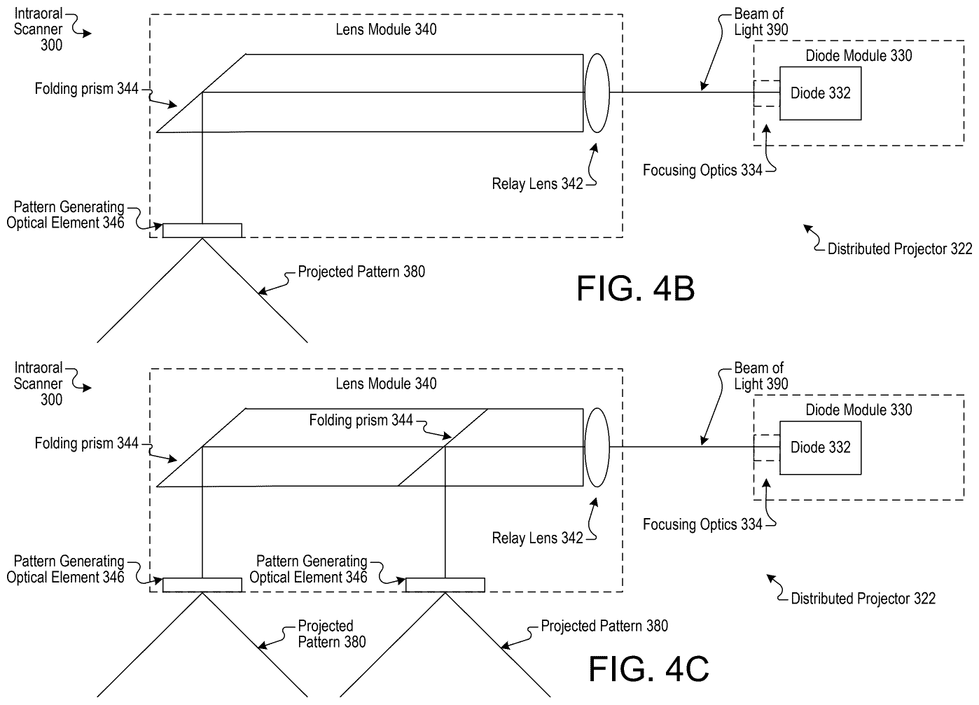

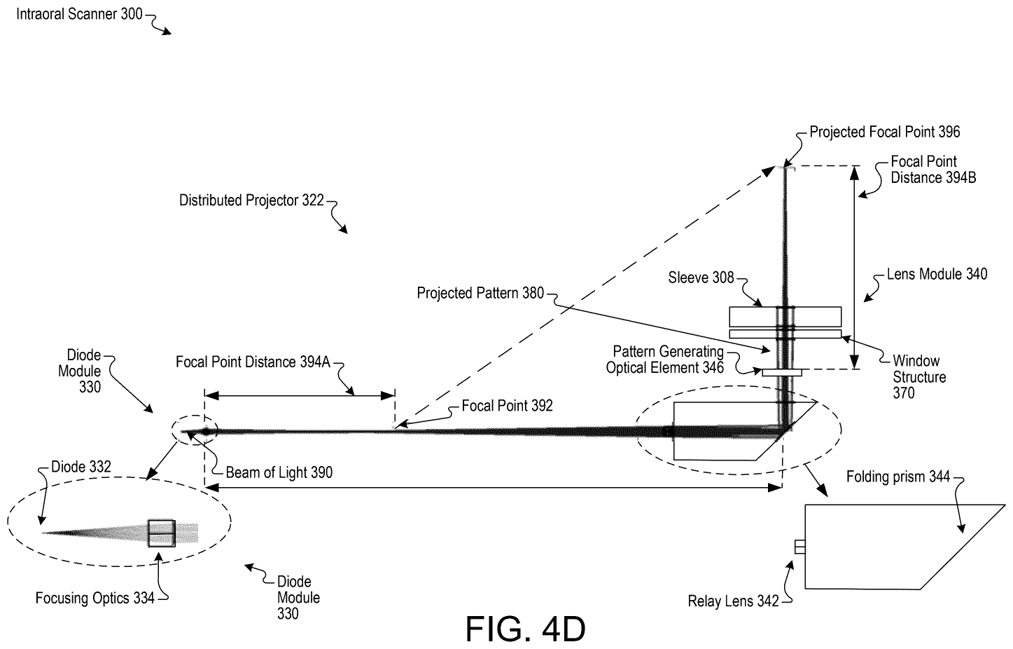

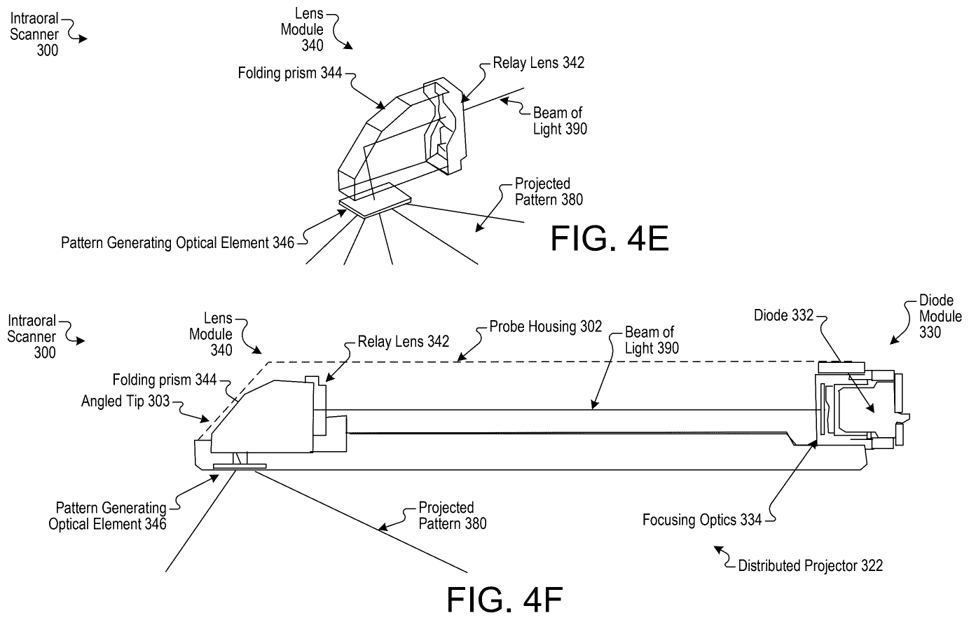

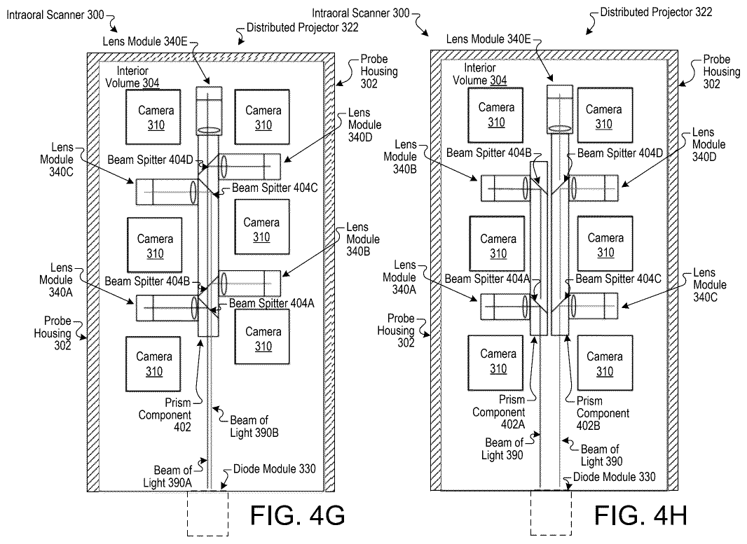

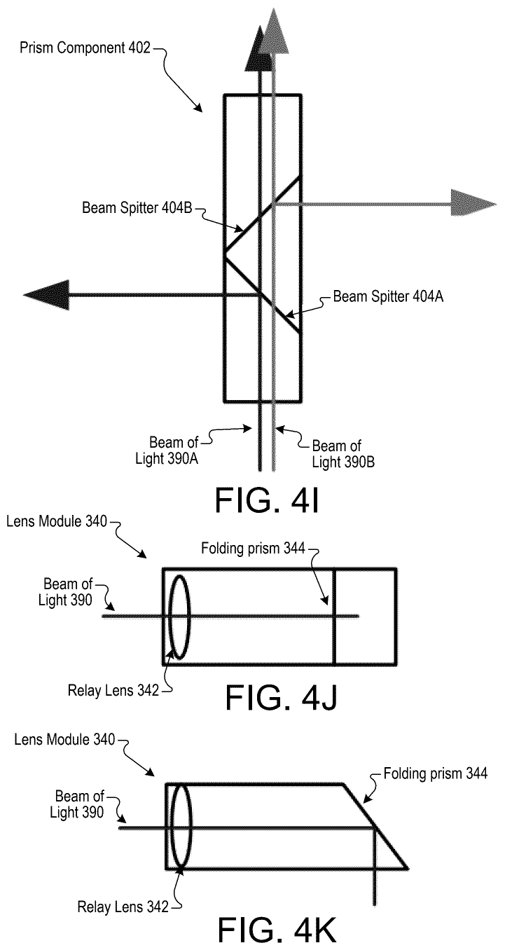

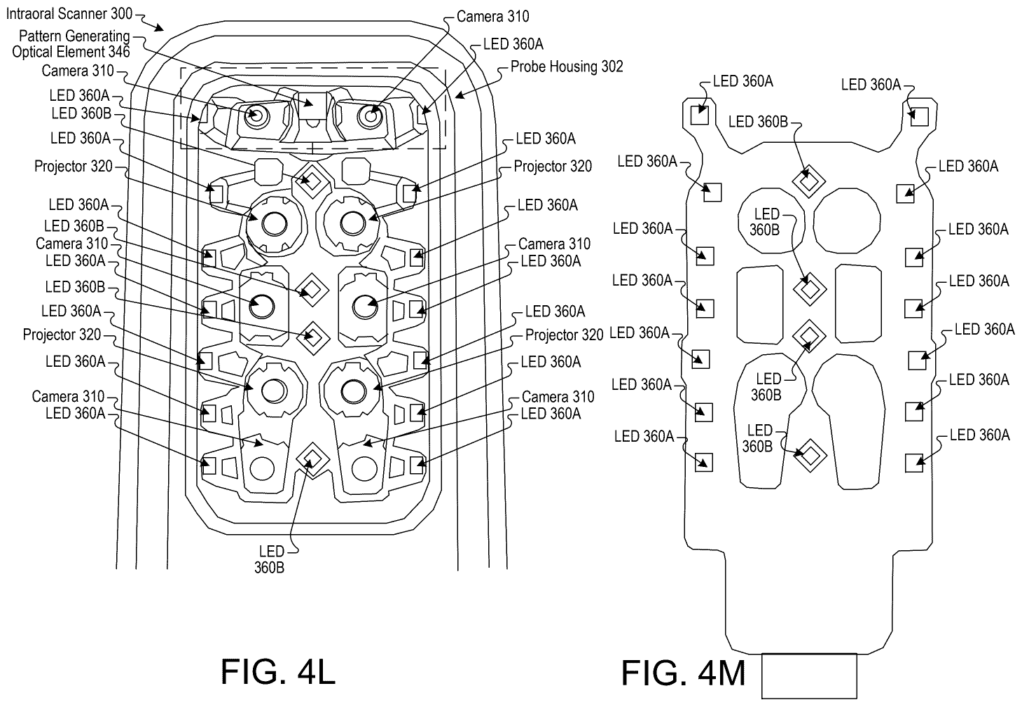

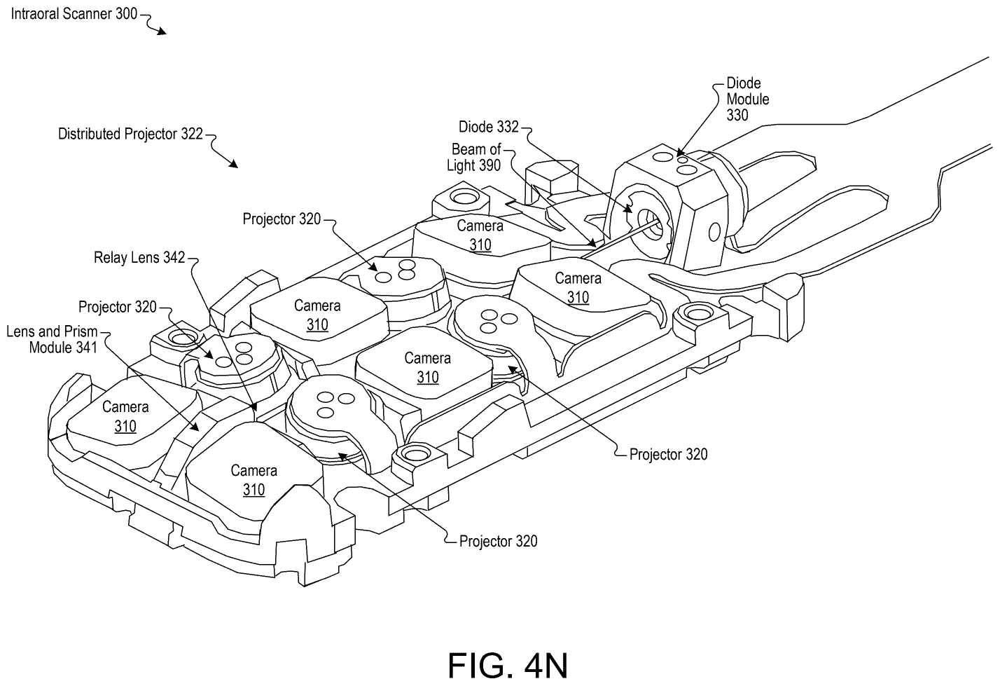

FIGS. 4A-O illustrate components of intraoral scanners 300 (e.g., that have one or more distributed projectors), according to certain embodiments. In some embodiments, intraoral scanners 300 of one or more of FIGS. 4A-O include similar or the same functionality, components, materials, and/or the like as scanner 150 of FIG. 1 and/or intraoral scanner 20 of FIGS. 2A-D.

In some embodiments, intraoral scanner 300 of one or more of FIGS. 4A-O includes one or more of the features of FIGS. 1-7.

In some embodiments, intraoral scanner 300 of one or more of FIGS. 4A-O includes: a probe housing disposed at a distal end of an elongate wand; a window structure coupled to the probe housing, the window structure and the probe housing forming an interior volume, wherein the window structure has a concave transverse cross section; and a plurality of optical components disposed within the interior volume, wherein the plurality of optical components comprise a first camera having a first orientation and a second camera having a second orientation, wherein the first camera and the second camera are to capture images of dental sites. In some embodiments, the plurality of optical components comprises a first pattern projector, a second pattern projector, and a camera disposed between the first pattern projector and the second pattern projector. In some embodiments, the first pattern projector and the second pattern projector are disposed within 5 millimeters of a foremost end of the probe housing. In some embodiments, each of the first pattern projector and the second pattern projector has a diameter of less than 4 millimeters and a height of less than 4.5 millimeters. In some embodiments, each of the first pattern projector and the second pattern projector has a diameter of about 2 millimeters and a height of about 3 millimeters. In some embodiments, the probe housing has a height of less than 10 millimeters and a width of less than 20 millimeters. In some embodiments, the probe housing has a height of about 7.5 to about 8.5 millimeters and a width of about 16 to about 17 millimeters. In some embodiments, the first orientation of the first camera and the second orientation of the second camera are configured to cause about 20% to about 40% overlap of image capture between the first camera and the second camera. In some embodiments, the first camera and the second camera are to capture images, wherein the images are to be used to perform model building via at least one of a correspondence algorithm or a trained machine learning model. In some embodiments, one or more of the plurality of optical components are configured to capture images of rearmost teeth in a mouth of a patient. In some embodiments, the first camera is to perform image capturing in the first orientation of a first side of the dental sites and the second camera is to perform image capturing in the second orientation of a second side of the dental sites to perform teeth wrapping imaging. In some embodiments, a first central axis of the first camera is orthogonal to a first portion of the window structure, and wherein a second central axis of the second camera is orthogonal to a second portion of the window structure.

In some embodiments, intraoral scanner 300 of one or more of FIGS. 4A-O includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, the plurality of optical components being bonded directly to the window structure. In some embodiments, the plurality of optical components comprises cameras and projectors. In some embodiments, the plurality of optical components are bonded directly to the window structure to provide drift-free retention of the plurality of optical components. In some embodiments, the plurality of optical components are bonded directly to the window structure via adhesive that is optically permeable.

In some embodiments, intraoral scanner 300 of one or more of FIGS. 4A-O includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, wherein the window structure comprises a first distal portion disposed at a first orientation and a second distal portion disposed at a second orientation that is at an angle to the first orientation, wherein the window structure is configured to block back reflection crosstalk by the plurality of optical components. In some embodiments, the plurality of optical components comprises cameras and projectors. In some embodiments, the window structure is a single piece of glass formed via glass molding. In some embodiments, the probe housing is wrapped by a heat sink metal structure that is in thermal contact with the probe housing. In some embodiments, a heating element is disposed between two or more of the plurality of optical components to heat the intraoral scanner. In some embodiments, the intraoral scanner further comprises a sleeve that includes an optical window, wherein the sleeve is configured to be removably disposed over the probe housing, wherein the optical window is configured to substantially align with the window structure. In some embodiments, the optical window of the sleeve comprises a first portion that approximately aligns with the first distal portion of the window structure, a second portion that approximately aligns with the second distal portion of the window structure, and a third portion that approximately aligns with a third portion of the window structure, the third portion of the window structure being disposed between the first distal portion and the second distal portion. In some embodiments, the window structure has a concave transverse cross section. In some embodiments, a first optical component of the plurality of optical components has a first axis that is orthogonal to the first distal portion of the window structure, wherein a second optical component of the plurality of optical components has a second axis that is orthogonal to the second distal portion of the window structure, and wherein a third optical component of the plurality of optical components has a third axis that is orthogonal to a third portion of the window structure, the third portion of the window structure being disposed between the first distal portion and the second distal portion. In some embodiments, the first optical component comprises a first structured light projector, wherein the second optical component comprises a camera, and wherein the third optical component comprises a second structured light projector. In some embodiments, the first optical component comprises a first camera, wherein the second optical component comprises a structured light projector, and wherein the third optical component comprises a second camera.

In some embodiments, the intraoral scanner of one or more of FIGS. 4A-O is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view of about 70 to about 90 degrees; and one or more non-object-facing imaging devices disposed within the interior volume in a rear portion of the probe housing, wherein the one or more non-object-facing imaging devices are configured to capture respective images via respective imaging axes that reflect to pass through the window structure, and wherein the one or more object-facing imaging devices have a second field of view of about 10 to about 30 degrees. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in the rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors. In some embodiments, the hybrid intraoral scanner further includes a fold mirror disposed at a distal end of the probe housing, wherein the fold mirror is configured to reflect the respective imaging axes and the respective projector axes to provide structured light 3D capture of a distal portion of a user mouth, and wherein the one or more non-object-facing imaging devices are oriented substantially parallel to the window structure. In some embodiments, the hybrid intraoral scanner further includes a prism disposed at a distal end of the probe housing, wherein the prism is configured to reflect the respective imaging axes and the respective projector axes to provide structured light 3D capture of a distal portion of a user mouth, wherein the prism has a fold angle of about 25 degrees to about 40 degrees, and wherein the one or more non-object-facing imaging devices are oriented substantially parallel to the window structure. In some embodiments, the hybrid intraoral scanner further includes a dichroic filter, wherein the one or more non-object-facing imaging devices comprise one or more upward-facing cameras that are oriented away from the window structure and an optical coherence tomography (OCT) channel that is oriented substantially parallel to the window structure, wherein the one or more upward-facing cameras are oriented upward and are configured to combine coaxially with a forward pointing OCT channel via the dichroic filter. In some embodiments, a first camera of the one or more object-facing imaging devices is configured to provide a viewfinder associated with moving the hybrid intraoral scanner. In some embodiments, In some embodiments, the one or more object-facing imaging devices and the one or more non-object-facing imaging devices are configured to provide imaging of one or more of a three-dimensional (3D) surface, subgingival, occlusal caries, proximal caries, or periodontal pockets. In some embodiments, the one or more object-facing imaging devices and the one or more non-object-facing imaging devices are configured to provide structured near-infrared (NIRI) images to provide shallow 3D viewing of hard tissue lesions. In some embodiments, the one or more object-facing imaging devices and the one or more object-facing projectors are configured to perform surface 3D capture. In some embodiments, the hybrid intraoral scanner further comprises an array of object-facing LEDs associated with two-dimensional color capture. In some embodiments, the one or more non-object-facing projectors comprise one or more of a rear NIRI structured light or NIRI collimated projector for subsurface 3D capture. In some embodiments, the one or more non-object-facing imaging devices comprise an OCT imaging channel coaxial configured for surface and sub-surface 3D structure capture. In some embodiments, the one or more non-object-facing imaging devices comprises an endoscopic OCT device inserted between the one or more object-facing imaging devices and the one or more object-facing projectors, wherein the endoscopic OCT device comprises a built-in rotating fold prism configured to provide transverse line scanning. In some embodiments, image data from the one or more non-object-facing imaging devices and the one or more object-facing imaging devices is to be combined to provide a third field of view that is greater than at least one of the first field of view or the second field of view. In some embodiments, the hybrid intraoral scanner further comprises a collimator, a scan mirror, and a fold mirror, wherein the one or more non-object-facing imaging devices comprise an OCT measuring arm fiber configured to provide light that is to pass through the collimator, reflect off the scan mirror, and reflect off the fold mirror to pass through the window structure.

In some embodiments, the intraoral scanner of one or more of FIGS. 4A-O is used with a method that includes: identifying first image data from one or more object-facing imaging devices at a first field of view of about 70 to about 90 degrees, the one or more object-facing imaging devices being disposed within an interior volume formed by a probe housing and a window structure of a hybrid intraoral scanner, the one or more object-facing imaging devices being disposed proximate the window structure; identifying second image data from one or more non-object-facing imaging devices at a second field of view of about 10 to about 30 degrees, the one or more non-object-facing imaging devices being disposed within the interior volume in a rear portion of the probe housing; and combining the first image data and the second image data. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in a rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors.

In some embodiments, the intraoral scanner of one or more of FIGS. 4A-O is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view; and an optical coherence tomography (OCT) imaging channel disposed within the interior volume in a rear portion of the probe housing, wherein the OCT imaging channel is configured to capture respective images via a respective imaging axis that reflects to pass through the window structure, and wherein the OCT imaging channel has a second field of view that is different from the first field of view. In some embodiments, the hybrid intraoral scanner further includes one or more object-facing projectors disposed within the interior volume proximate the window structure, wherein the one or more object-facing projectors provide corresponding light via corresponding projector axes that directly pass through the window structure. In some embodiments, the one or more object-facing devices and the one or more object-facing projectors are closer to a distal tip of the probe housing than the OCT imaging channel.

In some embodiments, the intraoral scanner of one or more of FIGS. 4A-O includes: a processing device configured to: receive a first image of an object from a first camera of the intraoral scanner, the first camera having a first static focal distance; determine a more sharp portion of the first image and a less sharp portion of the first image, the less sharp portion corresponding to a region of the object; for the less sharp portion of the first image, determine a more sharp portion of a second image corresponding to the region of the object, the second image being from a second camera of the intraoral scanner, the second camera having a second static focal distance that is different from the first static focal distance of the first camera; generate a combined image using the more sharp portion of the first image and the more sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface. In some embodiments, the processing device is further configured to receive a third image of the object from a third camera of the intraoral scanner; the third camera has a third static focal distance that is different from the first static focal distance of the first camera and the second static focal distance of the second camera; and the processing device is to generate the combined image further based on the third image. In some embodiments: the first camera is proximate a distal end of a tip of the intraoral scanner; the third camera is further from the distal end of a tip of the intraoral scanner than the first camera; the second camera is disposed between the first camera and the third camera; the first static focal distance of the first camera is less than the second static focal distance of the second camera; and the second static focal distance of the second camera is less than the third static focal distance of the third camera. In some embodiments, the combined image has an increased sharpness than the sharp portion of the first image and the sharp portion of the second image. In some embodiments, the first image of the object being captured by the first camera and the second image of the object being captured by the second camera in a similar location, in a similar angle, and at a similar brightness level. In some embodiments, the first camera and second camera capture images of the mouth of the user responsive to a light source of the intraoral scanner providing white light or near infrared light into the mouth of the user. In some embodiments, to generate the combined image, the processing device is to smooth the more sharp portion of the first image and the more sharp portion of the second image.

In some embodiments, the intraoral scanner of one or more of FIGS. 4A-O includes: a processing device configured to: receive a first image of an object from a first camera of the intraoral scanner, the first camera having a first static focal distance; receive a second image of the object from a second camera of the intraoral scanner, the second camera having a second static focal distance that is different from the first static focal distance; determine a first sharp portion of the first image corresponding to a first region of the object; determine a second sharp portion of the second image corresponding to the first region of the object; generate a combined image using the first sharp portion of the first image and the second sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface.

FIG. 4A illustrates a bottom cross-sectional view of an intraoral scanner 300. FIG. 4B illustrates a side view of components of an intraoral scanner 300. FIG. 4C illustrates a side view of components of an intraoral scanner 300. FIG. 4D illustrates a side view of components of an intraoral scanner 300. FIG. 4E illustrates a top perspective view of components of an intraoral scanner 300. FIG. 4F illustrates a side view of components of an intraoral scanner 300.

The intraoral scanner 300 may be a wand that is connected to a computation station. The tip of the intraoral scanner 300 may be inserted into the oral cavity of a person while scanning procedure is performed. A disposable sleeve may be placed over the tip of the intraoral scanner 300 prior to being inserted in the oral cavity. The tip of the intraoral scanner 300 may be part of an assembly that is separate from the rest of the wand. Once the assembly of the tip of the intraoral scanner 300 is completed, the tip can be added to the wand. The tip may connect to the wand via mechanical interfaces (e.g., screws, springs, bolts, fasteners) and electrical connections (e.g., connect to internal portion of scanner). The tip of the intraoral scanner 300 may be a standalone item that is later integrated with the rest of the intraoral scanner 300 (e.g., endpiece, scanner). The intraoral scanner 300 may use multi-structured light to create a 3D model. In some embodiments, the intraoral scanner 300 has cameras 310 (e.g., disposed in square holes formed by the intraoral scanner 300). The intraoral scanner 300 may have projectors 320 and/or portions of one or more distributed projectors 322 that are disposed adjacent to and/or between the cameras 310.

The projectors 320 may be non-distributed projectors. A non-distributed projector (e.g., projector 320) may include components (e.g., diode, focusing optics, relay lens, folding prism, pattern generating optical element, lens array, etc.) all disposed in one module (e.g., housing), that are bonded together, and/or that are less than a threshold distance from each other. The non-distributed projector (e.g., projector 320) may include components (e.g., disposed less than a threshold distance from each other) that are configured to emit a first beam of light and generate first structured light.

A distributed projector 322 includes at least two modules (e.g., housings), such as a diode module 330 including first components (e.g., diode 332, focusing optics 334) and a lens module 340 including second components (e.g., relay lens 342, folding prism 344, pattern generating optical element 346). Each of the modules (e.g., housings) may be disposed in different regions of the interior volume 304 of the probe housing 302. Each of the modules (e.g., housings) may not be bonded together and may be greater than a threshold distance from each other. The threshold distance may be at least 30 mm, at least 25 mm, at least 20 mm, at least 15 mm, at least 10 mm, or at least 5 mm.

In some embodiments, the lens module 340 is disposed at a distal end of the probe housing 302. In some embodiments, the distal end of the probe housing 302 has an angled tip that houses the lens module 340.

In some embodiments, a first set of projectors 320 and/or distributed projector 322 have a first type of illumination (e.g., blue illuminations) and a second set of projectors 320 and/or distributed projector 322 have a second type of illumination (e.g., green illumination). The multiple cameras 310 and multiple projectors 320 and/or one or more distributed projectors 322 may be used to provide sufficient data for 3D construction (e.g., 3D imaging) of the dental site.

In some embodiments, intraoral scanner 300 includes a probe housing 302 (e.g., thin stainless steel enclosure) disposed at a distal end of an elongate wand. The probe housing 302 may form an interior volume 304. The intraoral scanner 300 may include optical components disposed in the interior volume 304. The optical components may include cameras 310, projectors 320, and/or one or more distributed projectors 322.

In some embodiments, intraoral scanner 300 includes a distributed projector 322 disposed in in the interior volume 304 of probe housing 302. The distributed projector 322 may include a diode module 330 and a lens module 340. Diode module 330 and lens module 340 may be disposed at least a threshold distance (e.g., remote) from each other (e.g., one or more cameras 310 and/or non-distributed projectors 320 are disposed between the diode module 330 and the lens module 340). The lens module 340 may be disposed proximate a distal end of probe housing 302 (e.g., to illuminate rearmost teeth of a user). At least a portion of the cameras 310 are disposed between lens module 340 and diode module 330.

In some embodiments, the intraoral scanner 300 further includes a window (e.g., window structure) coupled to the probe housing 302. The lens module 340 is disposed at or proximate to a first distal end of the window and the diode module 330 is disposed at or proximate to a second distal end of the window.

In some embodiments, a window (e.g., window structure) is coupled to the probe housing 302. The window may cover the lens module 340 without covering the diode module 330.

In some embodiments, the lens module 340 is separated from the diode module 330 by air, an optical fiber, and/or a light guide.

Diode module 330 includes a diode 332 (e.g., laser diode) configured to emit a beam of light 390 and/or focusing optics 334 configured to focus the beam of light 390.

Lens module 340 includes a relay lens 342 configured to focus the beam of light 390, a folding prism 344 configured to deflect at least a first portion of the beam of light 390, and/or a pattern generating optical element 346 configured to generate, based on the at least a first portion of the beam of light 390, structured light (e.g., a projected pattern 380) to illuminate at least a portion of a mouth of a patient. In some embodiments, the pattern generating optical element 346 is at least one of a multi lens array (MLA) or a diffractive optical element (DOE).

In some embodiments, the lens module 340 includes a first folding prism 344 configured to deflect a first portion of the beam of light 390, a first pattern generating optical element 346 configured to generate, based on the first portion of the beam of light 390, first structured light (e.g., a first projected pattern 380), a second folding prism 344 configured to deflect a second portion of the beam of light 390, a second pattern generating optical element 346 configured to generate, based on the second portion of the beam of light 390, and a second structured light (e.g., a second projected pattern 380). The first folding prism 344 may be configured to transmit the second portion of the beam of light 390 to the second folding prism 344.

In some embodiments, the relay lens 342 is disposed between the diode module 330 and a folding prism 344 of the lens module 340. In some embodiments, the relay lens 342 is disposed between a first folding prism 344 of the lens module and the first pattern generating optical element 346. In some embodiments, the relay lens 342 is further configured to deflect the at least a first portion of the beam of light 390 to the first pattern generating optical element 346 (e.g., relay lens 342 and folding prism 344 are a single component).

In some embodiments, the focusing optics 334 is disposed between the diode 332 and the lens module 340. In some embodiments, the first pattern generating optical element 346 is disposed on a window structure 370 of the intraoral scanner 300. In some embodiments, intraoral scanner 300 includes cameras 310 disposed in a camera portion of the intraoral scanner 300, where the camera portion of the intraoral scanner 300 is disposed between the diode module 330 and the lens module 340. In some embodiments, the beam of light emitted from the diode module 330 is to pass between two or more cameras 310 and is to be received by the lens module 340.

In some embodiments, intraoral scanner 300 includes a first camera 310 and a second camera 310. The first camera 310 and the second camera 310 may be disposed in a distal portion of the intraoral scanner 300. The lens module 340 may be disposed between the first camera 310 and the second camera 310.

In some embodiments, the intraoral scanner has a hybrid approach of tip-mounted lens module 340 and back-mounted diode module 330 (e.g., in the back of the tip or the body of the intraoral scanner 300). The lens module 340 and the diode module 330 may be distributed portions of pattern projectors (e.g., structured light projectors). This allows a smaller size of the tip of the intraoral scanner 300 while maintaining high field of view (FOV) and structured light cover area. The distributed projector 322 splits the projector into the diode 332 (e.g., laser diode) with focusing optics 334, placed on the back fo the tip or the body of the scanner, and the pattern generating optical element 346 (e.g., MLA or DOE) in the tip. Another lens (e.g., relay lens 342) may be used in the midway to refocus the spots outside the intraoral scanner 300. A folding prism 344 is used to fold the optical path outside the intraoral scanner 300. This allows the FOV to be maintained while saving the space of the tip. The distributed projector 322 (e.g., split projector) may be referred to as a virtual projector (VP).

FIG. 4A illustrates one of multiple options of placing and arranging components in a hybrid structured light tip of an intraoral scanner 300. In some embodiments, the diode 332 (e.g., laser diode) emits a single beam of light 390 (e.g., ray of light) directed to a folding prism 344, which directs at least a portion of the beam of light 390 (e.g., at least a portion of the ray) onto the pattern generating optical element 346 (e.g., MLA, DOE). The pattern generating optical element 346 generates the structured light (e.g., projected pattern 380). The combination of the diode 332 (e.g., laser diode) and focusing optics 334 in a diode module 330 and the relay lens 342, folding prism 344, and pattern generating optical element 346 (e.g., MLA, DOE) in the lens module 340 forms the distributed projector 322 (e.g., split projector).

FIG. 4B illustrates an intraoral scanner 300 that has a lens module 340 that includes one folding prism 344 and one pattern generating optical element 346.

FIG. 4C illustrates an intraoral scanner 300 that has a lens module 340 that includes multiple folding prisms 344 (e.g., staggered prisms) and multiple pattern generating optical elements 346. By using staggered prisms, beam of light 390 from a single diode 332 (e.g., single laser diode) may be dropped off to multiple pattern generating optical elements 346.

By using a distributed projector 322 (e.g., hybrid approach), the dimensions of the tip of the intraoral scanner may be minimized. This is by splitting the projector into two parts, such as: diode 332 and focusing optics 334 on the back (where space is more available); and relay lens 342, folding prism 344, and pattern generating optical element 346 (which consume low space) in the front. In the multiple (e.g., staggered) folding prism 344 approach, one diode 332 can be used for multiple pattern generating optical elements 346 which further saves space.

FIG. 4D illustrates an intraoral scanner 300 that has a distributed projector 322 including a diode module 330 and a lens module 340. The diode module 330 may include a diode 332 and focusing optics 334. The lens module may include a relay lens 342, folding prism 344, and a pattern generating optical element 346. The pattern generating optical element 346 may be proximate the window structure 370 which is proximate the sleeve 308. The diode 332 emits a beam of light 390. The focusing optics 334 focuses the beam of light 390 to a focal point 392 that is a focal point distance 394A from the focusing optics 334. The relay lens 342 refocuses the beam of light 390. The folding prism 344 deflects the beam of light 390. The pattern generating optical element 346 generates, based on the beam of light 390, a projected pattern 380 that passes through the window structure 370 and the sleeve 308 across a focal point distance 394B to a projected focal point 396 (e.g., a dental site). In some embodiments, focal point distance 394A and focal point distance 394B are the same distance (or substantially the same distance. The focal point 392 may be the same distance (or substantially the same distance) from focusing optics 334 as projected focal point 396 is from pattern generating optical element 346. Focal point 392 may be an inner focal point. The distributed projector 322 may project the inner focal point 392 to projected focal point 396 outside of the tip window (e.g., window structure 370) (e.g., project projected focal point 396 to a same focal point distance 394B as focal point distance 394A).

FIG. 4E illustrates a lens module 340 of an intraoral scanner 300. Lens module 340 may include one or more of relay lens 342 to focus (e.g., re-focus) a beam of light 390, a folding prism 344 to deflect the beam of light 390, and a pattern generating optical element 346 to generate, based on the beam of light 390, a projected pattern 380.

FIG. 4F illustrates a distributed projector 322 of an intraoral scanner 300. The distributed projector 322 includes a diode module 330 that includes a diode 332 to emit a beam of light 390 and focusing optics 334 to focus the beam of light 390. The distributed projector 322 further includes a lens module 340 that includes a relay lens 342 to re-focus the beam of light 390, a folding prism 344 to deflect the beam of light 390, and a pattern generating optical element 346 to generate, based on the beam of light 390, a projected pattern 380. FIG. 4F may illustrate a probe housing 302.

The lens module 340 is disposed at a distal end of the probe housing 302. The distal end of the probe housing 302 has an angled tip 303 that houses the lens module 340 (e.g., the lens module is disposed in the angled tip 303. The angled tip 303 may have a lower height than a body of the probe housing 302.

In some embodiments, the cameras 310 are to capture images. The images are to be used to perform model building via a correspondence algorithm or machine learning. In some embodiments, the optical components (e.g., cameras 310) are configured to capture images of rearmost teeth in a mouth of a patient.

In some embodiments, the probe housing 302 forms an opening. The intraoral scanner may include a window structure 370 (e.g., structural window, optical window) coupled to the probe housing 302. The window structure 370 may cover the opening. The window structure 370 and the probe housing 302 may form the interior volume 304. The optical components (e.g., cameras 310, projectors 320, and/or distributed projector 322) may be disposed in the interior volume 304. The optical components (e.g., cameras 310, projectors 320, and/or lens module 340) may be bonded directly to the window structure 370 (via an adhesive, via an adhesive that is optically permeable, a high-level anti-contamination sealing adhesive). The optical components (e.g., cameras 310, projectors 320, and/or lens module 340) may be bonded directly to the window structure 370 to provide drift-free retention of the optical components.

In some embodiments, the intraoral scanner 300 includes a sleeve that includes an optical window (e.g., a sleeve window structure that is coupled to a sleeve housing). The sleeve may be configured to be removably disposed over the probe housing 302. The optical window of the sleeve may be configured to substantially align with the window structure 370 that is coupled to the probe housing 302. The intraoral scanner 300 (e.g., probe housing 302 and window structure 370) may be compatible with single piece disposable sleeve for cross contamination control.

The intraoral scanner 300 may have a reduced tip cross section compared to conventional scanners.

This may provide increased maneuverability. The optical components (e.g., cameras 310, lens module 340) of the intraoral scanner 300 may be closer to the tip distal end than conventional scanners. This may allow imaging of rearmost molars. The intraoral scanner 300 may have tighter camera-projector overlap than conventional scanners. This may improve image capture quality and coverage. The intraoral scanner 300 may have projectors 320, distributed projector 322, and cameras 310 distributed to provide triangulation diversity and reduced occlusions.

The intraoral scanner 300 may be inserted into a mouth of a patient until hitting a stop surface. The foremost capture aperture of the intraoral scanner 300 may reach deeper in a mouth of a patient than conventional scanners.

In some embodiments, probe housing 302 is a stainless steel enclosure. In some embodiments, the sleeve 308 is a transparent material and sleeve window 309 is the same material as the rest of the sleeve 308 (e.g., sleeve window 309 and sleeve housing of sleeve 308 are the same component). In some embodiments, sleeve window 309 is transparent and is different from the sleeve housing of the sleeve 308 (e.g., sleeve window 309 and sleeve housing of sleeve 308 are different components). An interface may couple the probe housing 302 to a wand body of the intraoral scanner 300.

U.S. patent application Ser. No. 18/226,651 to Atiya, et al., is assigned to the assignee of the present application, and is incorporated herein by reference, describes an ultraminiature pattern projector. One or more of projectors 320 may have the same or similar functionality, components, material, etc. as one or more of the ultraminiature pattern projectors described in U.S. patent application Ser. No. 18/226,651 to Atiya, et al. In some embodiments, each projector 320 (e.g., structured light projector) includes a housing, within which is disposed a light source. In some embodiments the housing is a sealed housing (e.g., is hermetically sealed). Each light source includes at least one semiconductor laser die and at least one beam shaping optical element. In some embodiments, the semiconductor laser die and the beam shaping optical element are disposed within a common chamber of the housing. Placing the beam shaping optical element and the semiconductor laser die of the structured light projector within the same chamber of the housing enables a distance between an emission point of the semiconductor laser die and an input face of the beam shaping optical element to be shorter than conventional laser diodes permit. Distance D between an emission point of the semiconductor laser diode and an input face of the beam shaping optical element is at least 50 microns and/or less than 250 microns. Some examples of the advantages provided are: overall reduction in size of the structured light projector, in turn enabling a reduction in size of the probe as well as increased flexibility in the arrangement of the structured light projectors and the cameras; increased collection efficiency of the laser light; increased depth of focus of the structured light projector; use of multiple laser dies within a single structured light projector, increasing the quantity of structured light features used for 3D reconstruction without increasing the size of and/or number of structured light projectors; and/or reduced speckle noise when using multiple laser dies.

Each projector 320 (e.g., structured light projector) may include at least one pattern generating optical element positioned such that each structured light projector projects a pattern of light onto an intraoral surface when the light source of the projector 320 is activated to emit light through the pattern generating optical element of the structured light projector.

An intraoral scanner 300 may include: an elongate handheld wand including a probe housing 302 at a distal end of the handheld wand; and one or more projectors 320 (e.g., structured light projectors) disposed within the probe housing 302, each projector 320 including a housing, a light source disposed within the housing, and a pattern generating optical element. The light source may include a semiconductor laser die and a beam shaping optical element. A distance D between an emission point of the semiconductor laser die and an input face of the beam shaping optical element is 50-250 microns. Each projector 320 is configured to project a pattern of light onto an intraoral surface when the light source of the structured light projector is activated to emit light through the pattern generating optical element of the structured light projector.

In some embodiments, the housing is a sealed housing. In some embodiments, the semiconductor laser die and the beam shaping optical element being disposed within the housing (e.g., within the sealed housing, within a common chamber of the housing) and distance D being 50-250 microns together allow a longest dimension of the sealed housing to be 1.5-2.5 mm.

In some embodiments, a height of the housing is 1.6-2.4 mm. In some embodiments, the beam shaping optical element is positioned within the housing such that at least 75% of the light emitted by the semiconductor laser die enters the beam shaping optical element. In some embodiments, the beam shaping optical element is positioned within the housing such that 80-90% of the light emitted by the semiconductor laser die enters the beam shaping optical element. In some embodiments, the housing includes metal and the semiconductor laser die is disposed within the housing such that heat is conducted from the semiconductor laser die to the metal of the housing. In some embodiments, the semiconductor laser die is mounted on a submount within the housing such that heat is conducted from the semiconductor laser die to the metal of the housing through the submount. In some embodiments, the submount is ceramic.

In some embodiments, the intraoral scanner 300 further includes one or more cameras 310 disposed within the probe housing 302, where a distance between (i) an optical axis of at least one camera 310 and (ii) an optical axis of at least one projector 320 (e.g., structured light projector) that is adjacent the at least one camera 310 is 3-5 mm.

U.S. patent application Ser. No. 17/869,698 to Atiya, et al., published as US20230025243A1 to Atiya, et. al, is assigned to the assignee of the present application, and is incorporated herein by reference, describes an intraoral scanner with illumination sequencing and controlled polarization. The intraoral scanner 300 may have the same or similar functionality, components, material, etc. as one or more of the embodiments described in U.S. patent application Ser. No. 18/226,651 to Atiya, et al. In some embodiments, a correspondence algorithm is used with the cameras 310 and the projectors 320 of intraoral scanner 300.

In some embodiments, each camera 310 includes a camera sensor that has an array of pixels, for each of which there exists a corresponding ray in 3-D space originating from the pixel whose direction is towards an object being imaged; each point along a particular one of these rays, when imaged on the sensor, will fall on its corresponding respective pixel on the sensor. The term used for this may be a “camera ray.” Similarly, for each projected spot from each projector 320 there exists a corresponding projector ray. Each projector ray corresponds to a respective path of pixels on at least one of the camera sensors, i.e., if a camera 310 sees a spot projected by a specific projector ray, that spot is detected by a pixel on the specific path of pixels that corresponds to that specific projector ray. Values for (a) the camera ray corresponding to each pixel on the camera sensor of each of the cameras, and (b) the projector ray corresponding to each of the projected spots of light from each of the projectors, may be stored during a calibration process.

In some embodiments, based on the stored calibration values a processing device may be used to run a correspondence algorithm in order to identify a 3D location for each projected spot on the surface. For a given projector ray, the processing device “looks” at the corresponding camera sensor path on one of the cameras 310. Each detected spot along that camera sensor path will have a camera ray that intersects the given projector ray. That intersection defines a 3D point in space. The processing device then searches among the camera sensor paths that correspond to that given projector ray on the other cameras 310 and identifies how many other cameras 310, on their respective camera sensor paths corresponding to the given projector ray, also detected a spot whose camera ray intersects with that 3D point in space. If two or more cameras 310 detect spots whose respective camera rays intersect a given projector ray at the same 3D point in space, the cameras 310 are considered to “agree” on the spot being located at that 3D point. Accordingly, the processing device may identify 3D locations of the projected light (e.g., projected) pattern of light) based on agreements of the two or more cameras 310 on there being the projected pattern of light by projector rays at certain intersections. The process is repeated for the additional spots along a camera sensor path, and the spot for which the highest number of cameras “agree” is identified as the spot that is being projected onto the surface from the given projector ray. A 3D position on the surface is thus computed for that spot.

In some embodiments, once a position on the surface is determined for a specific spot, the projector ray that projected that spot, as well as all camera rays corresponding to that spot, may be removed from consideration and the correspondence algorithm may be run again for a next projector ray. Ultimately, the identified 3D locations may be used to generate a digital 3D model of the intraoral surface.

U.S. patent application Ser. No. 17/869,698 to Atiya, et al., published as US20230025243A1 to Atiya, et. al, is assigned to the assignee of the present application, and is incorporated herein by reference, describes an intraoral scanner with illumination sequencing and controlled polarization. The intraoral scanner 300 may have the same or similar functionality, components, material, etc. as one or more of the embodiments described in U.S. patent application Ser. No. 18/226,651 to Atiya, et al. In some embodiments, a correspondence algorithm is used with the cameras 310 and the projectors 320 of intraoral scanner 300.

U.S. Patent Application No. 63/461,804 to Dafna, et al., is assigned to the assignee of the present application, and is incorporated herein by reference, describes determining 3D data for 2D points using machine learning. The intraoral scanner 300 may have the same or similar functionality, components, material, etc. as one or more of the embodiments described in U.S. Patent Application No. 63/461,804 to Dafna, et al. In some embodiments, machine learning is used with the cameras 310 and the projectors 320 of intraoral scanner 300 to determine 3D data (e.g., modeling of a dental arch) using 2D points.

In some embodiments, a method includes projecting, by one or more projectors 320 (e.g., structured light projectors) of an intraoral scanner 300, a light pattern including projector rays onto a dental site. The method may further include capturing, by cameras 310 of the intraoral scanner 300, images of at least a portion of the light pattern projected onto the dental site, where each camera 310 captures an image including points of at least the portion of the light pattern projected onto the dental site. The method may further include determining, for each projector ray, one or more candidate points that might have been caused by the projector ray.

In some embodiments, the method includes: processing information for each projector ray using a trained machine learning model, where the trained machine learning model generates one or more outputs including, for each projector ray, and for each candidate point associated with the projector ray, a probability that the candidate point corresponds to the projector ray; and determining 3D coordinates for at least some of the points in the images based on the one or more outputs of the trained machine learning model.

In some embodiments, the method includes: using a trained machine learning model to select candidate points for projector rays based on one or more inputs including probabilities of candidate points corresponding to projector rays; and determining 3D coordinates for at least some of the points in the images based on the selected candidate points for the plurality of projector rays.

In some embodiments, a method includes: using a first trained machine learning model to determine probabilities that captured points of a captured light pattern in one or more images correspond to projected points of a projected light pattern; using a second trained machine learning model to determine correspondence between a plurality of the captured points and the projected points based on one or more of the determined probabilities; and determining depth information for at least some of the plurality of captured points based on the determined correspondence.

In some embodiments, a method includes: using one or more trained machine learning models to determine correspondence between captured points of a captured light pattern in images and projected points of a projected light pattern; and determining depth information for at least some of the plurality of captured points based on the determined correspondence.

FIGS. 4G-K illustrate components of intraoral scanners 300 (e.g., that have one or more distributed projectors 322), according to certain embodiments. In some embodiments, intraoral scanners 300 of one or more of FIGS. 4G-K include similar or the same functionality, components, materials, and/or the like as scanner 150 of FIG. 1, intraoral scanner 20 of FIGS. 2A-D, and/or intraoral scanner 300 of FIGS. 4A-F. Intraoral scanners 300 may provide beams of light 390 of different wavelengths. For example, beam of light 390A may be 450 nanometers (nm) (blue) and beam of light 390B may be 520 nm (green).

In some embodiments, intraoral scanner 300 includes a probe housing 302 disposed at a distal end of an elongate wand. The probe housing 302 forms an interior volume 304.

A distributed projector 322 is disposed in the interior volume 304. The distributed projector 322 includes a diode module 330, lens modules 340, and one or more prism components 402. Each prism component 402 includes beam splitters 404 that may deflect at least a portion of a beam of light 390 of a first wavelength and may transmit (allow) at least a portion of a beam of light 390 of a second wavelength. The coating of the beam splitter 404 may deflect a portion of a first wavelength and transmit (allow) a second wavelength.

Referring to FIG. 4G, an intraoral scanner 300 may include a prism component 402 (e.g., one central glass prism) that includes beam splitters 404A-D. The prism component 402 may support two wavelengths with the internal beam splitters 404A-D that have beam splitter coatings. Diode module 330 (e.g., via one or more diodes) may emit a beam of light 390A (e.g., blue wavelength) and a beam of light 390B (e.g., green wavelength) to the prism component 402.

Beam splitter 404A may deflect a portion (e.g., about one third) of the beam of light 390A to a lens module 340A, transmit (allow) a portion (e.g., about two thirds) of the beam of light 390A, and may transmit (allow) the beam of light 390B.

Beam splitter 404B may deflect a portion (e.g., about half) of the beam of light 390B to a lens module 340B, may transmit (allow) a portion (e.g., about half) of the beam of light 390B, and may transmit (allow) the beam of light 390A (e.g., the remaining two thirds portion of beam of light 390A after beam splitter 404A reflected one third of beam of light 390A).

Beam splitter 404C may deflect a remaining portion (e.g., about half) of the beam of light 390B to a lens module 340C and may transmit (allow) the beam of light 390A (e.g., the remaining two thirds portion of beam of light 390A after beam splitter 404A reflected one third of beam of light 390A).

Beam splitter 404D may deflect a portion (e.g., about one third) of the beam of light 390A to a lens module 340D and transmit (allow) a portion (e.g., the remaining one third) of the beam of light 390A to a lens module 340E.

Referring to FIG. 4H, an intraoral scanner 300 may include a prism component 402A and a prism component 402B (e.g., two central glass prisms), each supporting a different wavelength. The prism components 402 may have beam splitters 404. The beam splitters 404 of FIG. 4H may have simpler coatings than the coatings of beam splitters 404 of FIG. 4G. In some embodiments, the intraoral scanner 300 of FIG. 4H may have a more symmetrical right-left of the tip than the intraoral scanner 300 of FIG. 4G.

Prism component 402A may have beam splitter 404A and beam splitter 404B. Prism component 402A may receive a beam of light 390A (e.g., of blue wavelength) from diode module 330. Beam splitter 404A may deflect a first portion (e.g., about half) of the beam of light 390A to lens module 340A and transmit (allow) a remaining portion (e.g., about half) of the beam of light 390A to beam splitter 404B. Beam splitter 404B may deflect a remaining portion (e.g., about half) of the beam of light 390A to lens module 340B.

Prism component 402B may have beam splitter 404C and beam splitter 404D. Prism component 402B may receive a beam of light 390B (e.g., of green wavelength) from diode module 330. Beam splitter 404C may deflect a first portion (e.g., about one third) of the beam of light 390B to lens module 340C and transmit (allow) a remaining portion (e.g., about two thirds) of the beam of light 390B to beam splitter 404D. Beam splitter 404D may deflect a portion (e.g., about one third) of the beam of light 390B to lens module 340D and transmit (allow) a remaining portion (e.g., about one third) of the beam of light 390B to lens module 340E.

Referring to FIGS. 4G-H, in some embodiments, the beam splitters 404 of prism components 402 are configured to provide substantially equal amounts of a beam of light 390 to different lens modules 340. In some embodiments, the beam splitters 404 of prism components 402 are configured to provide substantially different amounts of a beam of light 390 to different lens modules 340. In some embodiments, the beam splitters 404 of prism components 402 are adjustable to provide different amounts of a beam of light 390 to different lens modules 340. For example, a coating of a beam splitter 404 may transmit (allow) more or less of a beam of light 390 based on polarization of the coating. For example, responsive to a coating undergoing a first type of polarization, the beam splitter 404 may deflect the beam of light 390 and responsive to the coating undergoing a second type of polarization, the beam splitter 404 may transmit (allow) the beam of light 390. In some embodiments, the coating includes electro-optic crystal or liquid crystal that can alter the polarization. A processing device may electronically control the polarization (e.g., amount of beam of light 390 deflected and amount transmitted).

Each lens module 340 may have a relay lens configured to focus the first portion of the first beam of light, a folding prism configured to deflect the first portion of the first beam of light, and/or a pattern generating optical element configured to generate first structured light based on the first portion of the first beam of light.

In some embodiments, the same diode 332 is configured to emit beam of light 390A at a first wavelength and beam of light 390B at a second wavelength. In some embodiments, a first diode 332 is configured to emit beam of light 390A at a first wavelength and a second diode 332 is configured to emit beam of light 390B at a second wavelength. In some embodiments, the same diode module 330 (e.g., including one diode 332, including a first diode 332 and a second diode 332) is configured to emit beam of light 390A at a first wavelength and beam of light 390B at a second wavelength. In some embodiments, a first diode module 330 (e.g., including a first diode 332) is configured to emit beam of light 390A at a first wavelength and a second diode module 330 (e.g., including a second diode 332) is configured to emit beam of light 390B at a second wavelength.

FIG. 4I illustrates at least a portion of prism component 402 of FIG. 4G.

Beam splitter 404A may deflect a portion (e.g., about one third) of the beam of light 390A (e.g., to a lens module 340A), transmit (allow) a portion (e.g., about two thirds) of the beam of light 390A, and may transmit (allow) the beam of light 390B. Beam splitter 404A may partially reflect/transmit (ratio depends on design) wavelength of 450 nm (e.g., blue) and may be fully transmitting wavelength of 520 nm (e.g., green).

Beam splitter 404B may deflect a portion (e.g., about half) of the beam of light 390B (e.g., to a lens module 340B), may transmit (allow) a portion (e.g., about half) of the beam of light 390B, and may transmit (allow) the beam of light 390A (e.g., the remaining two thirds portion of beam of light 390A after beam splitter 404A reflected one third of beam of light 390A). Beam splitter 404B may fully transmit wavelength of 450 nm (e.g., blue) and may partially reflect/transmit (ratio depends on design) wavelength of 520 nm (e.g., green).

FIGS. 4J-K illustrate at least a portion of lens module 340 of FIGS. 4G-H. FIG. 4J illustrates a top view and FIG. 4K illustrates a side view. Lens module 340 may receive at least a portion of a beam of light 390 from a beam splitter 404. Relay lens 342 may focus (e.g., re-focus) the beam of light 390) and folding prism 344 may deflect the beam of light 390 to a pattern generating optical element (e.g., MLA, DOE). The folding prism 344 and the relay lens 342 may be an assembly.

FIGS. 4L-O illustrate components of an intraoral scanner 300. FIG. 4L illustrates an intraoral scanner 300 that includes a probe housing 302, cameras 310, projectors 320, and LEDs 360A-B. FIG. 4M illustrates the LEDs 460 (e.g., disposed on a PCB) of the intraoral scanner 300.

Intraoral scanner 300 may include a pattern generating optical element 346 (e.g., diffractive optical element). The pattern generating optical element 346 may be disposed proximate to or be part of a lens and prism module 341 (e.g., that includes a lens module 340 and/or folding prism 344).

The intraoral scanner 300 may provide easy access to scanning of teeth and improved distal molar reachability compared to conventional systems. The intraoral scanner 300 may have successful capture of margin line and deep ditches. The intraoral scanner 300 may have easier maneuvering than conventional systems (e.g., via edged tip for improved soft tissue displacement for anterior teeth scanning, thinner tip for improved buccal surfaces capture, etc.). The intraoral scanner 300 may have improved orientation and improved field of view (FOV) compared to conventional systems. The intraoral scanner 300 may provide a viewfinder in the center or front end and may provide stitching from short height which provides a clear and intuitive position to user (e.g., without fisheye effect).

A viewfinder refers to two cameras providing images and stitching (e.g., blending, merging) together the images to provide a view of a region of the mouth. By putting the images together, the combined image may remove specular reflections coming from the teeth (e.g., one image may have a first reflection in a first location, the second image may have a second reflection in a second location, and by merging the two images both reflections may be removed).

In some embodiments, the combined images of the viewfinder may be displayed to a user that is moving the intraoral scanner 300 in a mouth of a patient so that the user can know where the intraoral scanner is located and assist the user in moving and orienting the intraoral scanner 300 in the mouth of the patient. In some embodiments, the LEDs 360 (e.g., white LEDs 360A) may be outside the field of view of the cameras 310 (e.g., to avoid or prevent reflections in the images captured by the cameras 310). The LEDs 360 (e.g., white LEDs 360A) may provide substantially homogenous and unified illumination. In some embodiments, the intraoral scanner 300 includes greater than six, greater than eight, at least 10, greater than 10, greater than 12, or at least 14 white LEDs 360A (e.g., in the periphery, proximate edges of the window structure).

In some embodiments, the intraoral scanner 300 performs direct imaging (e.g., imaging axis of the cameras 310 is provided in a straight line from the camera 310 to the location being imaged via the window structure instead of being folded via a mirror).

In some embodiments, the combined images may be displayed via a user interface. Responsive to receiving user input of a region of a dental arch, a combined image of that region may be displayed via the user interface.

In some embodiments, the combined images are overlaid on a 3D model of a dental arch to providing surface texturing on the 3D model.

In some embodiments, the aspect ratios of the cameras 310 used for the viewfinder are longitudinal (e.g., cameras in the same row are longitudinal) and the combination of the images from the cameras 310 provides rectangular combined images. The cameras 310 may be closer together than conventional systems and the cameras 310 may not have a projector 320 between the cameras 310. The cameras 310 may be substantially parallel to each other (e.g., not tilted) and both may be facing down.

The intraoral scanner 300 may have two columns of optical elements (e.g., cameras 310 and projectors 320 in two columns). In some embodiments, cameras 310 and projectors 320 are unified (e.g., by a metal structure that secures the cameras 310 and projectors 320).

FIGS. 4N-O illustrate components of an intraoral scanner 300. FIG. 4N illustrates an intraoral scanner 300 including cameras 310, projectors 320, and a distributed projector 322 (e.g., distributed back projector). FIG. 4O illustrates an intraoral scanner 300 including a distributed projector 322 (e.g., distributed back projector).

Intraoral scanner 300 may include a lens and prism module 341 (e.g., that includes a lens module 340 and/or folding prism 344).

In some embodiments, the distal end (e.g., of the tip) of the intraoral scanner 300 has a pair of two distal short baseline cameras 310 and a projector (e.g., lens module of distributed projector 322) that enable better reachability and penetration than conventional systems.

In some embodiments, the intraoral scanner 300 has multiple columns of projectors 320 (e.g., structured light projectors, multi-structured light (MSL) projectors) and longitudinal/transversal baselines that enable capturability of diverse 3D points.

In some embodiments, the intraoral scanner 300 includes white LEDs 360A that enable diffused illumination and prevent saturated regions. The LEDs 360A may include an additional pair of white LEDs at the distal end (e.g., of the tip) of the intraoral scanner 300 which enables strong and uniform illumination for distal camera and distal teeth.

In some embodiments, the intraoral scanner 300 includes NIR LEDs 360B (e.g., occlusal infrared LEDs) located along a central portion of the intraoral scanner 300 (e.g., between cameras 310 and/or projectors 320). The NIR LEDs 360B may enable occlusal illumination.

In some embodiments, the intraoral scanner 300 includes parallel longitudinal field of view (FOV) mid cameras that may provide images that are to be merged into a unified viewfinder. In some embodiments, due to a short baseline, images from distal cameras 310 may be merged effectively. In some embodiments, the intraoral scanner 300 has less overlapping regions between the cameras 310 and the projectors 320 than conventional systems.

The last molar in a mouth of a patient may be located just before the mandibula. When the mouth is opened, the mastical muscle may be tight and solid. To acquire a 3D model of the dental arch of the patient, the tip of the intraoral scanner 300 may be small enough (e.g., meet a threshold height, tapered distal end of the tip of the intraoral scanner 300) to locate the distal two cameras 310 and lens module 340 of the distributed projector 322 in front of the third molar distal wall. The intraoral scanner may capture images at a further distance (e.g., an improved distal point of view, deeper penetration of the tip) in a mouth of the patient than conventional systems. The intraoral scanner 300 may have an improved distal point of view compared to conventional systems by bringing distal cameras and projector closer to the distal end of the intraoral scanner 300. This may provide more points at a larger field of view. The distal cameras 310 may have a larger FOV and the active area may be closer to the tip end of the intraoral scanner 300. The position of the optical components may allow for better buccal and lingual capture.

Conventionally, structured pattern of projectors and camera view may be shadowed by adjacent teeth. Spots of the structured pattern of projectors have an area on sharp edges where the spots of the structured pattern get split. The spots of the structured pattern may be swallowed by soft tissue and blood in the sub-gingival ditch.

The intraoral scanner 300 may have structured pattern of projectors 320 that provide greater uniformity (e.g., better signal-to-noise ratio, lower localization error, more spots in ditches), larger FOV (e.g., more spots in ditches), improved geometry (e.g., lower angles between cameras 310 and projectors 320 that provides less shadowing, spot patterns focused in the middle which provides more spots in ditches, multi-directional sources view which provides more spots in ditches), improved white light (e.g., more light, less shadowing, using white light for generating surfaces such as transitions like deep ditches and scan bodies), and/or improved spot quality base don tight focused projectors 320 than conventional systems.

The intraoral scanner 300 may have improved camera-projector coverage than conventional systems by spreading cameras 310 and projectors 320 into two columns (e.g., instead of a first column for cameras, a second column for projectors 320, and a third column for cameras 310) to improve the shadowing effect of teeth. The intraoral scanner may have improved penetration (e.g., short distal camera baseline) compared to conventional systems.

The intraoral scanner may provide a viewfinder to provide an intuitive, wide view, and centered viewfinder which provides user a clear orientation and positioning in the mouth. Cameras 310 are offset from the center and are not axial. Conventionally, stitching images from cameras 310 together is challenging and depends on orientation and positioning of the tip of the intraoral scanner 300 over the teeth.

The intraoral scanner 300 may utilize two center-positioned cameras 310 that are tilted (e.g., slightly tilted) toward each other to allow easier stitching of images from the cameras 310. The FOV of the cameras 310 may be longitudinal which allows larger FOV along (e.g., parallel to) the jaw instead of across the jaw. Distal cameras 310 may be used as the viewfinder due to a short baseline. In some embodiments, to provide the viewfinder at contact scan, both cameras 310 may be occlusal (e.g., centered over the tooth), a single camera 310 may be occlusal, the camera 310 may perform a deep buccal scan, the camera(s) 310 may be tilted over the edge of a tooth, and/or the like.

The intraoral scanner 300 may include two columns of optical components (e.g., alternating between camera 310 and projector 320). The intraoral scanner 300 may include rows of optical components (e.g., the rows may be substantially perpendicular to the columns). There may be rows that include cameras 310 (e.g., only cameras) and rows that include projectors 320 (e.g., only projectors). The intraoral scanner 300 may include a first row of distal-cameras (e.g., in the distal portion of the tip of the intraoral scanner), a second row of mid-cameras, and a third row of rear-cameras, where the second row is between the first row and the third row.

In some embodiments, the viewfinder is provided by the first row of cameras 310 (e.g., images from the distal-most cameras are stitched together to provide a view of where the intraoral scanner 300 is scanning in the mouth). In some embodiments, the viewfinder is provided by the second row of cameras 310 (e.g., images from the mid-cameras are stitched together to provide a view of where the intraoral scanner 300 is scanning in the mouth).

In some embodiments, the intraoral scanner 300 provides white light images which may provide for better images of margin lines and challenging geometries than conventional solutions. This improvement to white light images may enhance the 3D capabilities and color shading. This may allow the intraoral scanner 300 to have one or more of improved axial and lateral uniformity, less shadowing, better saturated objects, less specular reflections, less internal reflections, improved signal at distal cameras, etc.

The intraoral scanner 300 may have a pair of white light LEDs 360A located next to the distal end (e.g., by the first row of cameras 310) that may provide a strong and uniform illumination. This may provide clear and bright images of molar teeth based on eliminating yellowness effect and supporting white light distal molar feature.

The intraoral scanner 300 may have white light illumination based on two rows of small-size LEDs 360A allowing to double the amount of LEDs achieve a more uniform illumination and to increase the overall illumination without generating reflections. This may improve image quality by generating a diffused-like illumination to improve lateral uniformity. This may also reduce shadowing effect created by teeth. This may also provide distal effective illumination at close range.

The intraoral scanner 300 may have an improved dark palate signal compared to conventional systems. Diffused-like illumination may enable increase of white light energy and may collect more signal from long distance objects compared to conventional systems. This may minimize saturated regions based on distribution of illumination power to multiple small LEDs 360. This may allow better palate color recovery by minimizing saturated regions which may enable longer exposure and better signal-to-noise ratio at a longer distance compared to conventional systems.

In some embodiments, the intraoral scanner 300 includes white light LEDs 360A that provide an improved image quality by substantially no reflections (e.g., no reflections) at nominal (e.g., this may include assembly tolerances which may not observe any reflections). In some embodiments, the intraoral scanner 300 includes NIR LEDs 360B that have minimal reflection at nominal (e.g., minor NIR LED reflections would be observed in mid- and rear-cameras 310).

In some embodiments, the intraoral scanner 300 generates occlusal illumination by placing the NIR LEDs 360B at the center of the tip. In some embodiments, the intraoral scanner 300 may have higher frames per second (FPS) by using only a single NIR cycle. In some embodiments, the intraoral scanner 300 may enhance NIR contrast (e.g., at about 900 to 1000 nanometers (nm), at about 920 to about 960 nm, at about 940 nm, etc.).

In some embodiments, the intraoral scanner 300 has a distributed projector 322 (e.g., back-projector, back-projector system) to bring light (e.g., structured pattern) to the distal end of the intraoral scanner 300. The distributed projector 322 may have a diode module 330 (e.g., back projector) and a lens module 340. The diode module 330 may include a bare projector (e.g., no DOE). The lens module 340 may include a folding prism 344 (e.g., focusing prism). The folding prism 344 may be glued to the chassis (e.g., metal structure). The distributed projector 322 may be aligned within the intraoral scanner 300.

In some embodiments, the metal structure is secured and the distributed projector 322 is aligned to cause the beam of light 390 to cross the prism and cross the DOE. Adhesive material may be added to the diode module 330 and/or lens module 340 and may be sent to the heat chamber for curing (e.g., via ultra violet (UV) radiation).

The intraoral scanner 300 may include rear projection via the distributed projector 322 to provide additional spots (e.g., structured pattern of light) for the distal-cameras 310.

In some embodiments, the diode module 330 (e.g., diode 332) is actively aligned relative to the lens module 340 (e.g., relay lens 342, folding prism 344, pattern generating optical element 346). A single beam of light 390 emitted from the diode 332 of the diode module 330 may deviate from the reference mechanical datum of the diode 332 in a magnitude of up to 4 degrees (e.g., may not be able to rely on the diode 332 as an indication for beam direction). The mechanical datums of the diode 332 may be used as reference datums for the distributed projector 322. As a result, the distributed projector 322 may retain the beam deviation error of the diode 332 relative to the mechanical structure.

When mounting a deviated diode 332 in a precise mounting hole 492 of the mechanical structure 490, completely centric with the designated optical pupil of the relay lens, the beam fo light 390 emitted by the diode 332 may miss the designated location (e.g., designated pupil). This deviation is to be corrected so that the beam of light 390 reaches the pattern generating optical element 346 to generated the spot-array from the single beam of light 390. In some embodiments, to compensate for the beam deviation, a recessed hole 492 (e.g., oversized) is formed in the mechanical structure 490 (e.g., mounting part, where the diode 332 and/or diode module 330 is mounted). The oversized recessed hole 492 enables manipulating (e.g., tilting the diode 332 and/or diode module 330 so that the deviated beam of light 390 reaches the center of the desired optical pupil of the relay lens 342. The active alignment may be by tilting the deviated diode 332 inside the mounting hole until the beam reaches the middle of the optical pupil of the relay lens 342. Once the required result is achieved, the diode 332 secured (e.g., fixed, glued via dedicated adhesive, adhered, mechanically secured, fastened, welded, soldered, laser welded, bonded, etc.) in the hole 492. A jig (e.g., including up to five manipulators) may be used for the alignment procedure. The jig may allow moving the diode 332 in one or more of: ex and Oy (e.g., pivoting for the beam aligning as described above); x and y (e.g., compensating on the rotations pivots that are not completely collide with the beam exit point, creating undesired lateral travel); and/or z (e.g., to insert the projector into the mounting hole). Light provided by the diode 332 (e.g., a structured pattern, a patterned circled spot which is the alignment result indication) may appear on a target plate that is part of the jig (e.g., if the light provided by the diode 332 is in a particular location on the target plate, then the diode 332 is correctly oriented. The diode 332 may be secured by the metal structure. Adhesive may be applied through holes formed by the metal structure to secure the diode 332 in a particular orientation. After the addition of the adhesive, the jig may be placed in an oven for curing the adhesive. After curing, the mechanical structure of diode 332, mechanical structure 492 (e.g., mounting), relay lens 342, and folding prism 334 are released from the jig.

The diode 332 may include a diode structure including mechanical datums and a projector structure around the diode structure.

Referring to FIG. 4M, intraoral scanner 300 may include white LEDs 360A and NIR LEDs 360B. The white LEDs 360A may be in a first column proximate a first edge of the window structure of the intraoral scanner 300 and in a second column proximate a second edge of the window structure. The NIRI LEDs 360B may be in a third column that is between the first column and the second column. The two columns of white LEDs 360B proximate the edges of the window structure may reduce or prevent reflections. The LEDs 360 may be perpendicular to the scanning field of view.

In some embodiments, the folding prism 344 and the relay lens 342 are in a single component (e.g., single lens module 340).

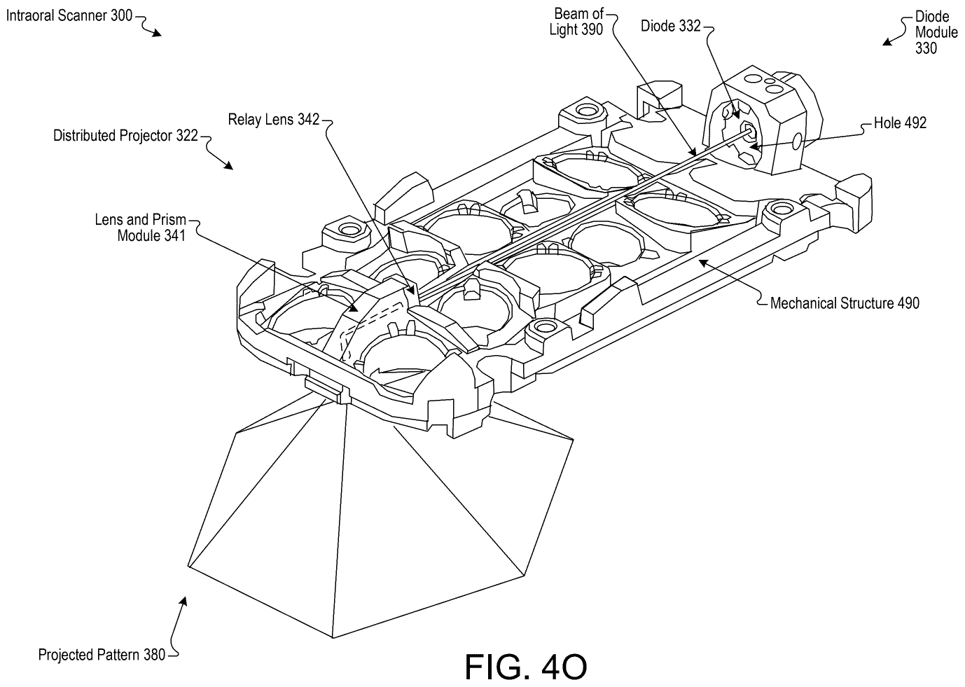

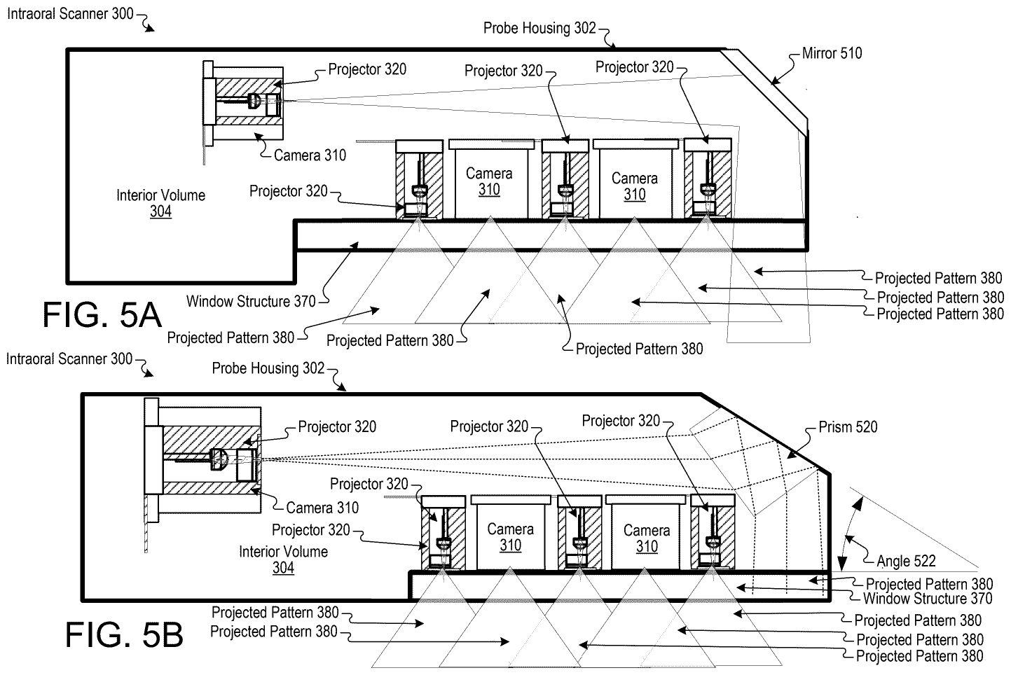

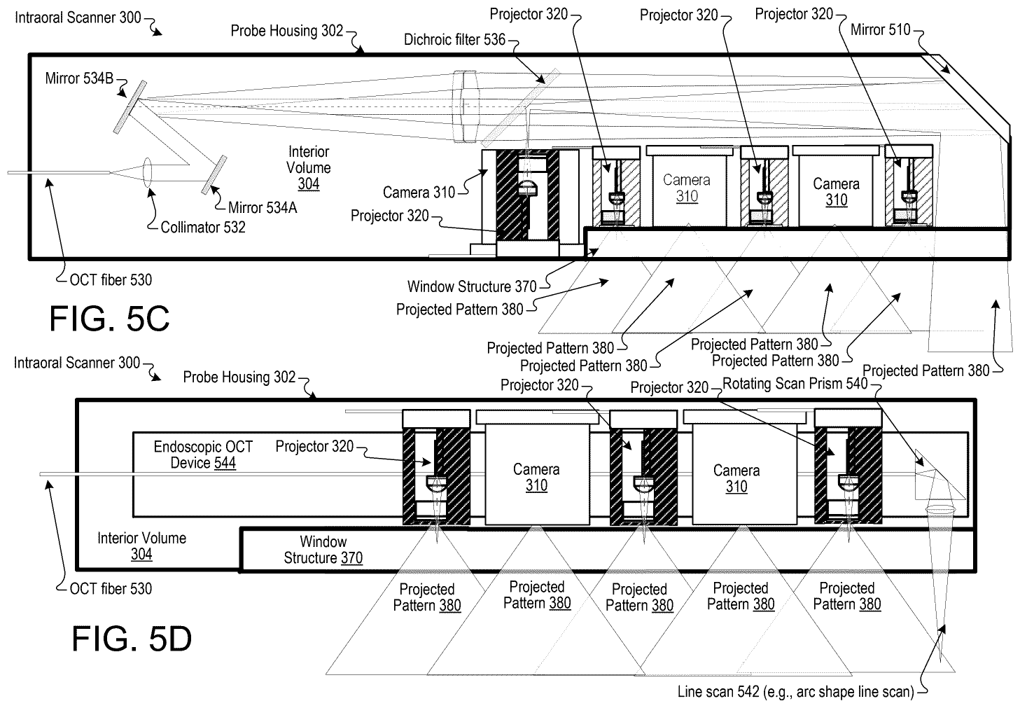

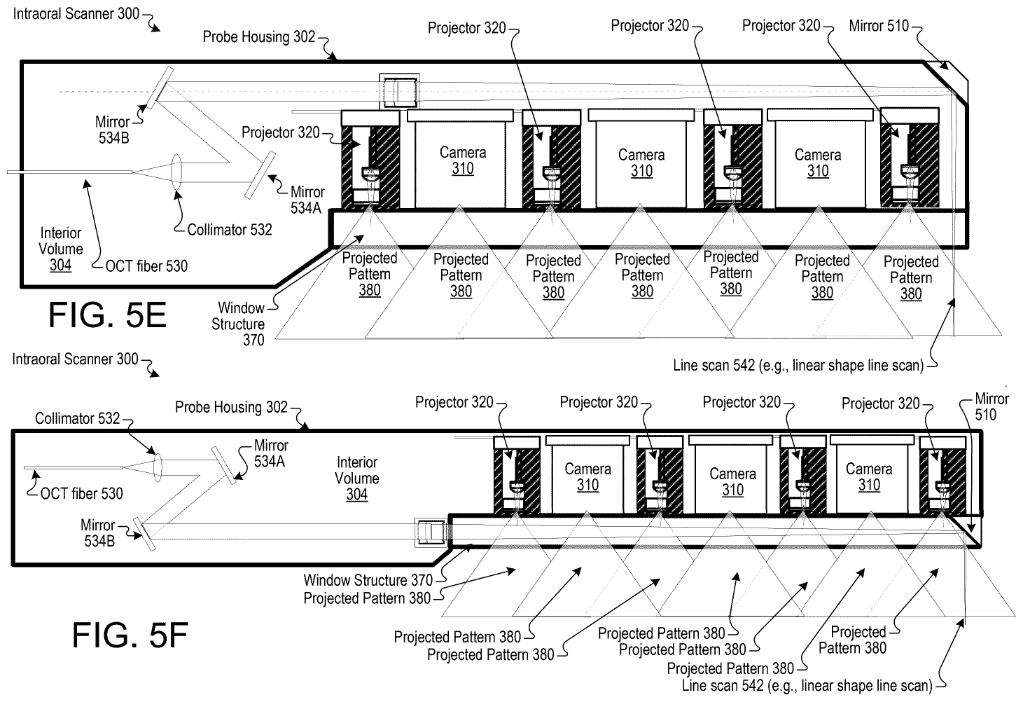

FIGS. 5A-F illustrate intraoral scanners 300 (e.g., hybrid intraoral scanners 300), according to certain embodiments.

In some embodiments, intraoral scanner 300 of one or more of FIGS. 5A-F includes one or more of the features of FIGS. 1-7.

In some embodiments, intraoral scanner 300 of one or more of FIGS. 5A-F includes: a probe housing disposed at a distal end of an elongate wand; a window structure coupled to the probe housing, the window structure and the probe housing forming an interior volume, wherein the window structure has a concave transverse cross section; and a plurality of optical components disposed within the interior volume, wherein the plurality of optical components comprise a first camera having a first orientation and a second camera having a second orientation, wherein the first camera and the second camera are to capture images of dental sites. In some embodiments, the plurality of optical components comprises a first pattern projector, a second pattern projector, and a camera disposed between the first pattern projector and the second pattern projector. In some embodiments, the first pattern projector and the second pattern projector are disposed within 5 millimeters of a foremost end of the probe housing. In some embodiments, each of the first pattern projector and the second pattern projector has a diameter of less than 4 millimeters and a height of less than 4.5 millimeters. In some embodiments, each of the first pattern projector and the second pattern projector has a diameter of about 2 millimeters and a height of about 3 millimeters. In some embodiments, the probe housing has a height of less than 10 millimeters and a width of less than 20 millimeters. In some embodiments, the probe housing has a height of about 7.5 to about 8.5 millimeters and a width of about 16 to about 17 millimeters. In some embodiments, the first orientation of the first camera and the second orientation of the second camera are configured to cause about 20% to about 40% overlap of image capture between the first camera and the second camera. In some embodiments, the first camera and the second camera are to capture images, wherein the images are to be used to perform model building via at least one of a correspondence algorithm or a trained machine learning model. In some embodiments, one or more of the plurality of optical components are configured to capture images of rearmost teeth in a mouth of a patient. In some embodiments, the first camera is to perform image capturing in the first orientation of a first side of the dental sites and the second camera is to perform image capturing in the second orientation of a second side of the dental sites to perform teeth wrapping imaging. In some embodiments, a first central axis of the first camera is orthogonal to a first portion of the window structure, and wherein a second central axis of the second camera is orthogonal to a second portion of the window structure.

In some embodiments, intraoral scanner 300 of one or more of FIGS. 5A-F includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, the plurality of optical components being bonded directly to the window structure. In some embodiments, the plurality of optical components comprises cameras and projectors. In some embodiments, the plurality of optical components are bonded directly to the window structure to provide drift-free retention of the plurality of optical components. In some embodiments, the plurality of optical components are bonded directly to the window structure via adhesive that is optically permeable.

In some embodiments, intraoral scanner 300 of one or more of FIGS. 5A-F includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, wherein the window structure comprises a first distal portion disposed at a first orientation and a second distal portion disposed at a second orientation that is at an angle to the first orientation, wherein the window structure is configured to block back reflection crosstalk by the plurality of optical components. In some embodiments, the plurality of optical components comprises cameras and projectors. In some embodiments, the window structure is a single piece of glass formed via glass molding. In some embodiments, the probe housing is wrapped by a heat sink metal structure that is in thermal contact with the probe housing. In some embodiments, a heating element is disposed between two or more of the plurality of optical components to heat the intraoral scanner. In some embodiments, the intraoral scanner further comprises a sleeve that includes an optical window, wherein the sleeve is configured to be removably disposed over the probe housing, wherein the optical window is configured to substantially align with the window structure. In some embodiments, the optical window of the sleeve comprises a first portion that approximately aligns with the first distal portion of the window structure, a second portion that approximately aligns with the second distal portion of the window structure, and a third portion that approximately aligns with a third portion of the window structure, the third portion of the window structure being disposed between the first distal portion and the second distal portion. In some embodiments, the window structure has a concave transverse cross section. In some embodiments, a first optical component of the plurality of optical components has a first axis that is orthogonal to the first distal portion of the window structure, wherein a second optical component of the plurality of optical components has a second axis that is orthogonal to the second distal portion of the window structure, and wherein a third optical component of the plurality of optical components has a third axis that is orthogonal to a third portion of the window structure, the third portion of the window structure being disposed between the first distal portion and the second distal portion. In some embodiments, the first optical component comprises a first structured light projector, wherein the second optical component comprises a camera, and wherein the third optical component comprises a second structured light projector. In some embodiments, the first optical component comprises a first camera, wherein the second optical component comprises a structured light projector, and wherein the third optical component comprises a second camera.

In some embodiments, the intraoral scanner 300 of one or more of FIGS. 5A-F includes: a probe housing disposed at a distal end of an elongate wand, the probe housing forming an interior volume; and a distributed projector disposed in the interior volume, the distributed projector comprising: a diode module comprising a laser diode configured to emit a beam of light; and a lens module comprising a first pattern generating optical element configured to generate structured light based on at least a first portion of the beam of light, the lens module being disposed at least a threshold distance from the diode module. In some embodiments, the first pattern generating optical element is at least one of a multi lens array (MLA) or a diffractive optical element (DOE). In some embodiments, the lens module further comprises a first folding prism configured to deflect the at least a first portion of the beam of light to the first pattern generating optical element. In some embodiments, the lens module further comprises a second folding prism and a second pattern generating optical element; the first folding prism is configured to transmit a second portion of the beam of light to the second folding prism; and the second folding prism is configured to deflect the second portion of the beam of light to the second pattern generating optical element. In some embodiments, the lens module further comprises a relay lens configured to focus the beam of light. In some embodiments, the relay lens is disposed between the diode module and a folding prism of the lens module. In some embodiments, the relay lens is disposed between a first folding prism of the lens module and the first pattern generating optical element. In some embodiments, the relay lens is further configured to deflect at least the first portion of the beam of light to the first pattern generating optical element. In some embodiments, the diode module further comprises focusing optics configured to focus the beam of light, the focusing optics being disposed between the laser diode and the lens module. In some embodiments, the first pattern generating optical element is disposed on a window structure of the intraoral scanner. In some embodiments, the intraoral scanner further comprises a plurality of cameras disposed in a camera portion of the intraoral scanner, wherein the camera portion of the intraoral scanner is disposed between the diode module and the lens module. In some embodiments, the beam of light emitted from the diode module is to pass between two or more cameras and is to be received by the lens module. In some embodiments, the intraoral scanner further comprises a first camera and a second camera, the first camera and the second camera being disposed in a distal portion of the intraoral scanner, wherein the lens module is disposed between the first camera and the second camera. In some embodiments, the intraoral scanner further comprises: a window coupled to the probe housing, wherein the lens module is disposed at or proximate to a first distal end of the window and the diode module is disposed at or proximate to a second distal end of the window. In some embodiments, the intraoral scanner further comprises a window coupled to the probe housing, wherein the window covers the lens module and does not cover the diode module. In some embodiments, the lens module is separated from the diode module by air, an optical fiber, or a light guide. In some embodiments,

In some embodiments, the intraoral scanner of one or more of FIGS. 5A-F includes: a probe housing disposed at a distal end of an elongate wand, the probe housing forming an interior volume; and a distributed projector disposed in the interior volume, the distributed projector comprising: a diode module configured to emit a first beam of light at a first wavelength; and a first prism component comprising: a first beam splitter configured to deflect a first portion of the first beam of light and transmit a second portion of the first beam of light; and a second beam splitter configured to deflect the second portion of the first beam of light. In some embodiments, the intraoral scanner further comprises: a lens module disposed at least a threshold distance from the diode module, the lens module comprising: a relay lens configured to focus the first portion of the first beam of light; a folding prism configured to deflect the first portion of the first beam of light; and a pattern generating optical element configured to generate first structured light based on the first portion of the first beam of light. In some embodiments, the diode module is further configured to emit a second beam of light at a second wavelength. In some embodiments, the intraoral scanner further comprises: a second prism component comprising: a third beam splitter configured to deflect a first portion of the second beam of light and transmit a second portion of the second beam of light; and a fourth beam splitter configured to deflect the second portion of the second beam of light. In some embodiments, the first beam splitter and the second beam splitter are further configured to transmit the second beam of light, wherein the first prism component further comprises: a third beam splitter configured to deflect a first portion of the second beam of light and transmit a second portion of the second beam of light; and a fourth beam splitter configured to deflect the second portion of the second beam of light. In some embodiments, the distributed projector is of an intraoral scanner, the distributed projector includes: a diode module comprising a laser diode configured to emit a beam of light; and a lens module disposed at least a threshold distance from the diode module, the lens module comprising: a folding prism configured to deflect at least a first portion of the beam of light; and a pattern generating optical element configured to generate, based on the at least a first portion of the beam of light, structured light to illuminate at least a portion of a mouth of a patient. In some embodiments, the diode module further comprises focusing optics configured to focus the beam of light. In some embodiments, the lens module further comprises a relay lens configured to focus the beam of light. In some embodiments,

In some embodiments, the intraoral scanner of one or more of FIGS. 5A-F includes: a probe housing disposed at a distal end of an elongate wand, the probe housing forming an interior volume; a non-distributed projector disposed in the interior volume; and a distributed projector disposed in the interior volume, the distributed projector comprising a diode module and a lens module. In some embodiments: the non-distributed projector comprising components configured to emit a first beam of light and generate first structured light; the components are less than a threshold distance from each other; the diode module is configured to emit a second beam of light; the lens module is configured to generate second structured light based on at least a portion of the second beam of light; and the lens module is disposed greater than the threshold distance from the diode module. In some embodiments, the lens module is disposed at a distal end of the probe housing. In some embodiments, the distal end of the probe housing has an angled tip that houses the lens module.

In some embodiments, the intraoral scanner of one or more of FIGS. 5A-F includes: a processing device configured to: receive a first image of an object from a first camera of the intraoral scanner, the first camera having a first static focal distance; determine a more sharp portion of the first image and a less sharp portion of the first image, the less sharp portion corresponding to a region of the object; for the less sharp portion of the first image, determine a more sharp portion of a second image corresponding to the region of the object, the second image being from a second camera of the intraoral scanner, the second camera having a second static focal distance that is different from the first static focal distance of the first camera; generate a combined image using the more sharp portion of the first image and the more sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface. In some embodiments, the processing device is further configured to receive a third image of the object from a third camera of the intraoral scanner; the third camera has a third static focal distance that is different from the first static focal distance of the first camera and the second static focal distance of the second camera; and the processing device is to generate the combined image further based on the third image. In some embodiments: the first camera is proximate a distal end of a tip of the intraoral scanner; the third camera is further from the distal end of a tip of the intraoral scanner than the first camera; the second camera is disposed between the first camera and the third camera; the first static focal distance of the first camera is less than the second static focal distance of the second camera; and the second static focal distance of the second camera is less than the third static focal distance of the third camera. In some embodiments, the combined image has an increased sharpness than the sharp portion of the first image and the sharp portion of the second image. In some embodiments, the first image of the object being captured by the first camera and the second image of the object being captured by the second camera in a similar location, in a similar angle, and at a similar brightness level. In some embodiments, the first camera and second camera capture images of the mouth of the user responsive to a light source of the intraoral scanner providing white light or near infrared light into the mouth of the user. In some embodiments, to generate the combined image, the processing device is to smooth the more sharp portion of the first image and the more sharp portion of the second image.

In some embodiments, the intraoral scanner of one or more of FIGS. 5A-F includes: a processing device configured to: receive a first image of an object from a first camera of the intraoral scanner, the first camera having a first static focal distance; receive a second image of the object from a second camera of the intraoral scanner, the second camera having a second static focal distance that is different from the first static focal distance; determine a first sharp portion of the first image corresponding to a first region of the object; determine a second sharp portion of the second image corresponding to the first region of the object; generate a combined image using the first sharp portion of the first image and the second sharp portion of the second image; and cause, based on the combined image, an action associated with a mouth of a user. In some embodiments, the action associated with the mouth of the user comprises one or more of caries detection, margin line detection, interproximal area detection, or texture mapping of a two-dimensional image on a three-dimensional model, or displaying image data of portions of the mouth of the user via a review tool graphical user interface.

In some embodiments, intraoral scanner 300 is a compact intraoral scanner that has multiple modalities that use different imaging configurations, some of which are 3D. The intraoral scanner 300 includes tip mounted, object facing, structured light 3D imaging devices, rear structural imaging devices without, or with, coaxial rear NIR structural imaging devices and 2D color imaging devices. The imaging devices, such as cameras 310, sensors, and light sources (e.g., projectors 320) may span a broad range of wavelengths from about 420 nm to about 1350 nm. This broad range of imaging components provides extended intraoral diagnostics capability.

Intraoral scanner 300 may be a multi-configuration scanner. Intraoral scanner may have rear structured light (e.g., non-object facing projectors 320). Intraoral scanner may include tip-mounted structured light (e.g., object-facing projectors 320). Intraoral scanner may have hybrid structured light (e.g., non-object facing projectors 320 and object-facing projectors 320). Intraoral scanner may include an optical coherence tomography (OCT) scanner.

In some embodiments, multi device/imaging configurations integrated in the same compact/lightweight handheld intraoral scanner 300 may provide concurrent multimodal oral imaging.

In some embodiments, intraoral scanner 300 may have a compact multiconfiguration tip having wide FOV that results in better global accuracy and higher speed.

In some embodiments, intraoral scanner 300 allows for several provisions for single camera viewfinder (e.g., distal cameras 310, mid cameras 310, rear cameras).

In some embodiments, intraoral scanner 300 is used for performing diagnostics of one or more of 3D surface, subgingival, occlusal and proximal caries, cancer, periodontal pockets, etc.

In some embodiments, structured NIR light of intraoral scanner 300 provides shallow 3D viewing of hard tissue lesions.

In some embodiments, intraoral scanner 300 includes object-facing cameras 310 and object-facing structured light projectors 320 to perform surface 3D capture.

In some embodiments, intraoral scanner 300 includes an array of object-facing white LEDs 360A for 2D color capture.

In some embodiments, intraoral scanner 300 includes an array of object-facing NIR structured light projectors for subsurface 3D capture.

In some embodiments, intraoral scanner 300 includes a rear NIR structured light and/or NIR uniform/collimated projectors for subsurface 3D capture.

In some embodiments, intraoral scanner 300 includes rear-mounted projectors 320 and cameras 310 to capture images of objects through a fold mirror for distal 3D capture.

In some embodiments, intraoral scanner 300 includes an OCT imaging channel and rear-mounted projectors 320 and cameras 310 to perform surface and sub-surface 3D structure capture.

In some embodiments, the intraoral scanner 300 (e.g., hybrid intraoral scanner) includes a probe housing 302 disposed at a distal end of an elongate wand and a window structure 370 coupled to the probe housing. The window structure 370 and the probe housing 302 form an interior volume 304 of the intraoral scanner 300. The intraoral scanner 300 further includes one or more object-facing imaging devices (e.g., downward-facing cameras) disposed within the interior volume 304 proximate the window structure 370 (e.g., adjacent to the window structure 370, secured to the window structure 370, configured to capture images directly through the window structure 370). The one or more object-facing imaging devices are configured to capture corresponding images via corresponding imaging axes that directly pass through the window structure 370. The one or more object-facing imaging devices have a first field of view (e.g., above about 45 degrees, of about 70 to about 90 degrees, of about 80 degrees.).

The intraoral scanner 300 further includes one or more non-object-facing imaging devices (e.g., rear-mounted cameras, upward facing cameras, OCT fiber) disposed within the interior volume 304 in a rear portion of the probe housing 302. The one or more non-object-facing imaging devices are configured to capture respective images via respective imaging axes that reflect (e.g., fold via a mirror 510 or prism 520) to pass through the window structure 370. The one or more object-facing imaging devices have a second field of view (e.g., of about 10 to about 30 degrees) that is different than (e.g., smaller than) the first field of view. The smaller field of view may provide a higher resolution and/or a higher penetration angle.

In some embodiments, the object-facing imaging devices (e.g., cameras 310) and the non-object facing imaging devices (e.g. OCT fiber, rear-mounted cameras 310, and/or upward-facing cameras 310) scan the mouth of the patient simultaneously (e.g., substantially simultaneously).

Object-facing may refer to being oriented with a central axis (e.g., projecting axis, image capturing axis) that is substantially perpendicular to the window structure 370. Object-facing may refer to being oriented with a central axis that passes directly through the window structure 370 (e.g., without being reflected or folded by a mirror or prism). Object-facing may refer to downward-facing (e.g., facing toward the window structure 370, providing light or capturing images directly through the window structure 370).

Non-object facing may refer to being oriented with a central axis (e.g., projecting axis, image capturing axis) that is substantially parallel to or extends away from the window structure 370. Non-object facing may refer to being oriented with a central axis that does not pass directly through the window structure 370 (e.g., is reflected or folded by a mirror or prism before passing through the window structure 370). Non-object-facing may refer to being rear-mounted or upward-facing (e.g., facing away from the window structure 370).

In some embodiments, the intraoral scanner 300 further includes one or more object-facing projectors disposed within the interior volume 304 proximate the window structure 370. The one or more object-facing projectors provide corresponding light via corresponding projector axes that directly pass through the window structure 370. The object-facing projectors may have a FOV (e.g., of about 80 to about 100 degrees, about 90 degrees) that is similar to the FOV of the object facing cameras (e.g., about 70 to about 90 degrees, about 80 degrees).

The intraoral scanner 300 further includes one or more non-object-facing projectors disposed within the interior volume 304 in the rear portion of the probe housing 302. The one or more non-object-facing projectors provide respective light via respective projector axes that reflect to pass through the window structure 370. The non-object-facing projectors may include a projector and a pattern generating optical element (e.g., DOE, MLA) that are proximate to each other (e.g., light beam from non-object-facing projector passes through the pattern generating optical element prior to being reflected by mirror 510 or prism 520).

In some embodiments, the respective light of the one or more non-object-facing projectors is provided via a pattern generating optical element (e.g., pattern generating optical element that is proximate the rear-mounted or upward-facing projector) prior to being reflected to pass through the window structure 370.

In some embodiments, the one or more object-facing devices and the one or more object-facing projectors are closer to a distal tip of the probe housing 304 than the one or more non-object-facing imaging devices and the one or more non-object-facing projectors.

In some embodiments, a first camera of the one or more object-facing imaging devices is configured to provide a viewfinder associated with moving the intraoral scanner 300 (e.g., hybrid intraoral scanner). In some embodiments, a row of two cameras (e.g., distal cameras, mid-cameras, rear cameras) of the one or more object-facing imaging devices is configured to provide a viewfinder associated with moving the hybrid intraoral scanner.

In some embodiments, the one or more object-facing imaging devices and the one or more non-object-facing imaging devices are configured to provide imaging of one or more of a three-dimensional (3D) surface, subgingival, occlusal caries, proximal caries, or periodontal pockets.

In some embodiments, the one or more object-facing imaging devices and the one or more non-object-facing imaging devices are configured to provide structured near-infrared (NIRI) images to provide shallow 3D viewing of hard tissue lesions.

In some embodiments, the one or more object-facing imaging devices and the one or more object-facing projectors are configured to perform surface 3D capture.

In some embodiments, the intraoral scanner 300 further includes an array of object-facing LEDs associated with two-dimensional color capture.

In some embodiments, the one or more non-object-facing projectors comprise one or more of a rear NIRI structured light or NIRI collimated projector for subsurface 3D capture.

In some embodiments, image data from the one or more non-object-facing imaging devices and the one or more object-facing imaging devices is to be combined to provide a third field of view that is greater than at least one of the first field of view or the second field of view.

In some embodiments, a method includes identifying first image data from one or more object-facing imaging devices at a first field of view (e.g., about 70 to about 90 degrees). The one or more object-facing imaging devices are disposed within an interior volume 304 formed by a probe housing 302 and a window structure 370 of the intraoral scanner 300 (e.g., hybrid intraoral scanner). The one or more object-facing imaging devices are disposed proximate the window structure 370. The method further includes identifying second image data from one or more non-object-facing imaging devices at a second field of view (e.g., about 10 to about 30 degrees). The one or more non-object-facing imaging devices are disposed within the interior volume 304 in a rear portion of the probe housing 302. The method further includes combining the first image data and the second image data.

In FIGS. 5A-B, the one or more non-object-facing imaging devices may include one or more rear-mounted cameras 310.

FIG. 5A is a longitudinal cross-section of an intraoral scanner 300 that includes a mirror 510. The intraoral scanner 300 may include projectors 320 and cameras 310 (e.g., rear-mounted cameras 310 and projectors 320) disposed at the rear of the tip or wand providing structured light 3D capture of the distal portion via a mirror (e.g., 45-degree fold mirror). Being disposed in the rear, these optical components allow for improved reach to last molars due to their slacker perspective. The large FOV is maintained when the rear-mounted projectors 320 and cameras 310 are combined with object-facing cameras 310 and projectors 320 of the same intraoral scanner 300.

In some embodiments, the intraoral scanner 300 further includes a mirror 510 (e.g., a fold mirror) disposed at a distal end of the probe housing 304. The fold mirror 510 is configured to reflect the respective imaging axes and the respective projector axes to provide structured light 3D capture of a distal portion of a user mouth. The one or more non-object-facing imaging devices are oriented substantially parallel to the window structure 370.

The non-object facing components may include at least one rear-mounted projector and at least one or two rear-mounted cameras 310.

FIG. 5B is a longitudinal cross-section of an intraoral scanner 300 that includes a prism 520 (e.g., intraoral scanner that has slack fold). The intraoral scanner 300 may include projectors 320 and cameras 310 (e.g., rear-mounted cameras 310 and projectors 320) disposed at the rear of the tip or wand providing structured light 3D capture of the distal portion via a prism 520 (e.g., a small prism yielding a slacker fold angle 522 which may be about 32 degrees). As with FIG. 5A, these rear-mounted optical components allow for improved reach to last molars due to their slacker perspective. The large FOV is maintained when the later are combined with object-facing cameras and projectors.

In some embodiments, the intraoral scanner 300 further includes a prism 520 disposed at a distal end of the probe housing. The prism 520 is configured to reflect the respective imaging axes and the respective projector axes to provide structured light 3D capture of a distal portion of a user mouth. The prism has a fold angle of about 25 degrees to about 40 degrees (e.g., about 32 degrees). The one or more non-object-facing imaging devices are oriented substantially parallel to the window structure 370.

In FIGS. 5C-F, the one or more non-object facing imaging devices may include an OCT fiber 530 (e.g., OCT fiber 530 plus structured light via downward-facing projectors 320). The OCT fiber 530 may provide light that has a FOV that is a line (e.g., instead of an area). This allows creating a low tip profile. The structured light from object-facing projectors 320 is used to combine the multiple line scans by the OCT fiber 530. The OCT fiber 530 may provide optical coherence tomography (e.g., optical section imaging, imaging beneath the skin, imaging a cross-sectional view).

The OCT fiber may be used for multimodal intraoral scanning as described in U.S. application Ser. No. 17/813,555, filed on Jul. 19, 2022, the contents of which are incorporated by reference in their entirety.

An intraoral scanner 300 (e.g., hybrid intraoral scanner of FIGS. 5C-F) may include a probe housing 302 disposed at a distal end of an elongate wand and a window structure 370 coupled to the probe housing 304. The window structure 370 and the probe housing 304 form an interior volume 304. The intraoral scanner 300 further includes one or more object-facing imaging devices disposed within the interior volume 304 proximate the window structure 370. The one or more object-facing imaging devices are configured to capture corresponding images via corresponding imaging axes that directly pass through the window structure 370. The one or more object-facing imaging devices have a first field of view (e.g., of about 70 to about 90 degrees, of about 80 degrees.). The intraoral scanner 300 further includes an OCT fiber 530 (e.g., OCT imaging channel) disposed within the interior volume 304 in a rear portion of the probe housing 302. The OCT imaging channel is configured to capture respective images via a respective imaging axis that reflects to pass through the window structure. The OCT imaging channel has a second field of view that is different from the first field of view.

In some embodiments, the intraoral scanner 300 further includes one or more object-facing projectors 320 disposed within the interior volume 304 proximate the window structure 370. The one or more object-facing projectors 320 provide corresponding light via corresponding projector axes that directly pass through the window structure.

In some embodiments, the intraoral scanner 300 further includes one or more non-object-facing projectors 320 (e.g., upward facing projectors) disposed within the interior volume 304 in a rear portion of the probe housing 302. The one or more non-object-facing projectors 320 provide respective light via respective projector axes that reflect to pass through the window structure 370.

In some embodiments, the one or more object-facing devices (e.g., downward-facing cameras 310) and the one or more object-facing projectors 320 are closer to a distal tip of the probe housing 302 than the Oct fiber 530 (e.g., OCT imaging channel).

FIG. 5C is a longitudinal cross-section of an intraoral scanner 300 that includes an OCT fiber 530 (e.g., intraoral scanner that has multi-structured light with a coaxial OCT plus 2D color imaging). In FIG. 5C, the non-object facing imaging devices may include one or more upward-facing cameras 310 and an OCT fiber 530.

The intraoral scanner 300 may include a set of projectors 320 and cameras 310 disposed at the rear of the tip or wand providing structured light 3D capture of the distal portion via a mirror 510 (e.g., a small 45 degrees fold mirror). As shown in FIG. 5C, the set of projectors 320 and cameras 310 may be upwards and combined coaxially with a forward pointing OCT channel (e.g., OCT fiber 530) via a dichroic filter 536. As with the intraoral scanner 300 of FIG. 5B, the rear devices allow for improved reach to last molars due to their slacker perspective. This configuration also enables subsurface imaging using the OCT channel (e.g., OCT fiber 530). The large FOV is maintained when the non-object facing optical components are combined with object-facing cameras 310 and projectors 320.

The OCT fiber 530 may provide light via a collimator, mirrors 534A-B (e.g., dual axis MEMS scan mirrors), dichroic filter 536, and mirror 510 and may receive the reflection of the light back through the same components from mirror 510 to dichroic filter 536 to mirrors 534A-B to collimator to the OCT fiber 530.

In some embodiments, the intraoral scanner 300 further includes a dichroic filter 536. The one or more non-object-facing imaging devices include one or more upward-facing cameras that are oriented away from the window structure 370 and an optical coherence tomography (OCT) channel (e.g., OCT fiber 530) that is oriented substantially parallel to the window structure. The one or more upward-facing cameras are oriented upward and are configured to combine coaxially with a forward pointing OCT channel via the dichroic filter 536.

In some embodiments, the one or more non-object-facing imaging devices include an OCT fiber 530 (e.g., OCT imaging channel coaxial) configured for surface and sub-surface 3D structure capture.

FIG. 5D is a longitudinal cross-section of an intraoral scanner 300 that includes an OCT fiber 530 (e.g., intraoral scanner that has multi-structured light with a rotating line OCT). The intraoral scanner 300 may include an endoscopic OCT device 544 (e.g., endoscopic OCT tip) inserted between the object-facing optical components. The endoscopic OCT device has built-in rotating fold prism 540 that is used as a transverse line scanning component. The longitudinal scan axis is manifested by the longitudinal scanner motion activated by the user. The speed, position, and orientation information is provided by the object-facing optical components. The OCT fiber 530 and rotating scan prism 540 are used to provide a line scan 542 (e.g., arc shape line scan).

In some embodiments, the one or more non-object-facing imaging devices include an endoscopic OCT device 544 inserted between the one or more object-facing imaging devices and the one or more object-facing projectors 320. The endoscopic OCT device 544 includes a built-in rotating fold prism 540 configured to provide transverse line scanning.

FIG. 5E is a longitudinal cross-section of an intraoral scanner 300 that includes an OCT fiber 530 (e.g., transverse single axis OCT with structured light). The intraoral scanner 300 may include an OCT fiber 530 (e.g., a single line OCT channel) disposed at the rear of the tip and directed towards the object by a mirror 510 (e.g., a 45 degrees mirror). The longitudinal scan axis is manifested by the longitudinal scanner motion activated by the user. The large FOV is maintained when the OCT fiber 530 is combined with object-facing cameras 310 and projectors 320. The OCT fiber 530 (e.g., single line OCT channel) uses the speed, position, and orientation information provided by the object-facing optical components. The OCT fiber 530, collimator 532, mirrors 534A-B, and mirror 510 are used to provide a line scan 542 (e.g., linear shape line scan).

In some embodiments, the intraoral scanner further includes a collimator 532, a scan mirror 534, and a fold mirror 510. The one or more non-object-facing imaging devices include an OCT fiber (e.g., an OCT measuring arm fiber) configured to provide light that is to pass through the collimator 532, reflect off the scan mirror 534, and reflect off the fold mirror 510 to pass through the window structure 370.

FIG. 5F is a longitudinal cross-section of an intraoral scanner 300 that includes an OCT fiber 530 (e.g., structured light with transverse OCT through window structure 370). The intraoral scanner 300 may include an OCT fiber 530 (e.g., a single line OCT channel) disposed at the rear of the tip propagating through the window structure 370 (e.g., optical window) and directed towards the object by a mirror 510 (e.g., 45 degree mirror) located at the distal end of the window structure 370. The longitudinal scan axis is manifested by the longitudinal scanner motion activated by the user. The large FOV is maintained when the OCT fiber 530 is combined with object-facing cameras 310 and projectors 320. The OCT fiber 530 (e.g., single line OCT channel) uses the speed, position and orientation information provided by the object-facing optical components. The OCT fiber 530, collimator 532, mirrors 534A-B, window structure 370, and mirror 510 are used to provide a line scan 542 (e.g., linear shape line scan).

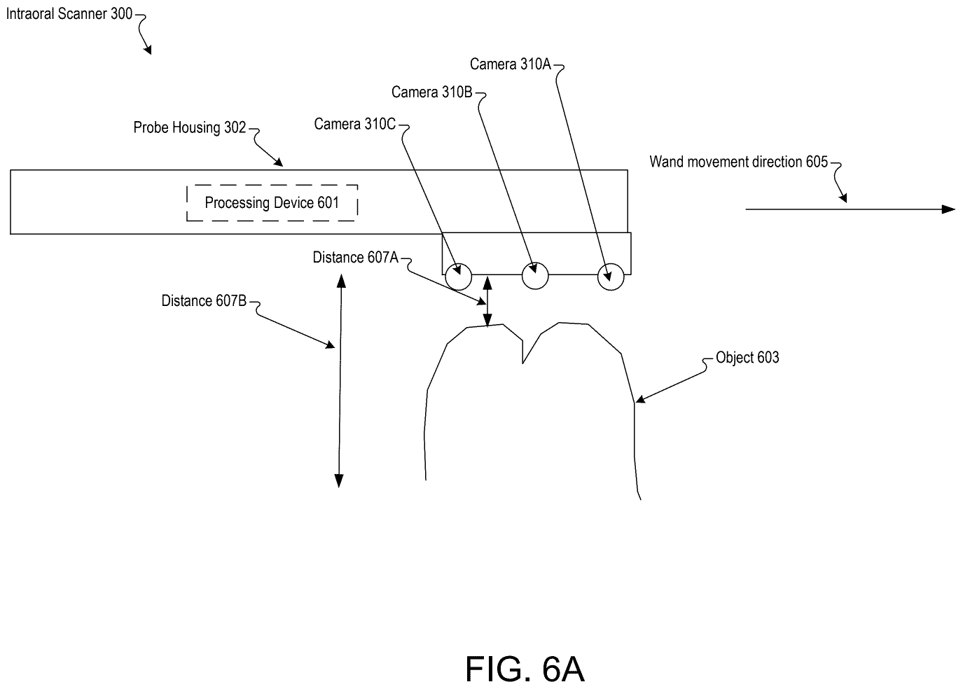

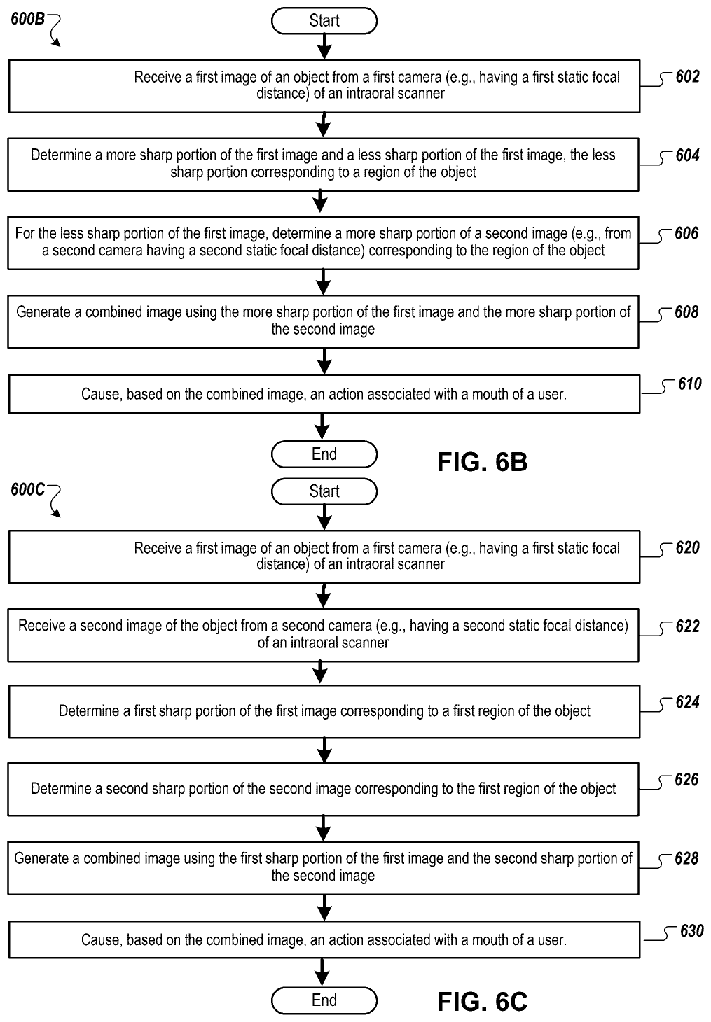

FIG. 6A illustrates an intraoral scanner 300 that has different object focus distance for different cameras 310.

In some embodiments, intraoral scanner 300 of one or more of FIGS. 6A-C includes one or more of the features of FIGS. 1-7.

In some embodiments, intraoral scanner 300 of one or more of FIGS. 6A-C includes: a probe housing disposed at a distal end of an elongate wand; a window structure coupled to the probe housing, the window structure and the probe housing forming an interior volume, wherein the window structure has a concave transverse cross section; and a plurality of optical components disposed within the interior volume, wherein the plurality of optical components comprise a first camera having a first orientation and a second camera having a second orientation, wherein the first camera and the second camera are to capture images of dental sites. In some embodiments, the plurality of optical components comprises a first pattern projector, a second pattern projector, and a camera disposed between the first pattern projector and the second pattern projector. In some embodiments, the first pattern projector and the second pattern projector are disposed within 5 millimeters of a foremost end of the probe housing. In some embodiments, each of the first pattern projector and the second pattern projector has a diameter of less than 4 millimeters and a height of less than 4.5 millimeters. In some embodiments, each of the first pattern projector and the second pattern projector has a diameter of about 2 millimeters and a height of about 3 millimeters. In some embodiments, the probe housing has a height of less than 10 millimeters and a width of less than 20 millimeters. In some embodiments, the probe housing has a height of about 7.5 to about 8.5 millimeters and a width of about 16 to about 17 millimeters. In some embodiments, the first orientation of the first camera and the second orientation of the second camera are configured to cause about 20% to about 40% overlap of image capture between the first camera and the second camera. In some embodiments, the first camera and the second camera are to capture images, wherein the images are to be used to perform model building via at least one of a correspondence algorithm or a trained machine learning model. In some embodiments, one or more of the plurality of optical components are configured to capture images of rearmost teeth in a mouth of a patient. In some embodiments, the first camera is to perform image capturing in the first orientation of a first side of the dental sites and the second camera is to perform image capturing in the second orientation of a second side of the dental sites to perform teeth wrapping imaging. In some embodiments, a first central axis of the first camera is orthogonal to a first portion of the window structure, and wherein a second central axis of the second camera is orthogonal to a second portion of the window structure.

In some embodiments, intraoral scanner 300 of one or more of FIGS. 6A-C includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, the plurality of optical components being bonded directly to the window structure. In some embodiments, the plurality of optical components comprises cameras and projectors. In some embodiments, the plurality of optical components are bonded directly to the window structure to provide drift-free retention of the plurality of optical components. In some embodiments, the plurality of optical components are bonded directly to the window structure via adhesive that is optically permeable.

In some embodiments, intraoral scanner 300 of one or more of FIGS. 6A-C includes: a probe housing disposed at a distal end of an elongate wand, the probe housing forming an opening; a window structure coupled to the probe housing, the window structure covering the opening, the window structure and the probe housing forming an interior volume; and a plurality of optical components disposed within the interior volume, wherein the window structure comprises a first distal portion disposed at a first orientation and a second distal portion disposed at a second orientation that is at an angle to the first orientation, wherein the window structure is configured to block back reflection crosstalk by the plurality of optical components. In some embodiments, the plurality of optical components comprises cameras and projectors. In some embodiments, the window structure is a single piece of glass formed via glass molding. In some embodiments, the probe housing is wrapped by a heat sink metal structure that is in thermal contact with the probe housing. In some embodiments, a heating element is disposed between two or more of the plurality of optical components to heat the intraoral scanner. In some embodiments, the intraoral scanner further comprises a sleeve that includes an optical window, wherein the sleeve is configured to be removably disposed over the probe housing, wherein the optical window is configured to substantially align with the window structure. In some embodiments, the optical window of the sleeve comprises a first portion that approximately aligns with the first distal portion of the window structure, a second portion that approximately aligns with the second distal portion of the window structure, and a third portion that approximately aligns with a third portion of the window structure, the third portion of the window structure being disposed between the first distal portion and the second distal portion. In some embodiments, the window structure has a concave transverse cross section. In some embodiments, a first optical component of the plurality of optical components has a first axis that is orthogonal to the first distal portion of the window structure, wherein a second optical component of the plurality of optical components has a second axis that is orthogonal to the second distal portion of the window structure, and wherein a third optical component of the plurality of optical components has a third axis that is orthogonal to a third portion of the window structure, the third portion of the window structure being disposed between the first distal portion and the second distal portion. In some embodiments, the first optical component comprises a first structured light projector, wherein the second optical component comprises a camera, and wherein the third optical component comprises a second structured light projector. In some embodiments, the first optical component comprises a first camera, wherein the second optical component comprises a structured light projector, and wherein the third optical component comprises a second camera.

In some embodiments, the intraoral scanner 300 of one or more of FIGS. 6A-C includes: a probe housing disposed at a distal end of an elongate wand, the probe housing forming an interior volume; and a distributed projector disposed in the interior volume, the distributed projector comprising: a diode module comprising a laser diode configured to emit a beam of light; and a lens module comprising a first pattern generating optical element configured to generate structured light based on at least a first portion of the beam of light, the lens module being disposed at least a threshold distance from the diode module. In some embodiments, the first pattern generating optical element is at least one of a multi lens array (MLA) or a diffractive optical element (DOE). In some embodiments, the lens module further comprises a first folding prism configured to deflect the at least a first portion of the beam of light to the first pattern generating optical element. In some embodiments, the lens module further comprises a second folding prism and a second pattern generating optical element; the first folding prism is configured to transmit a second portion of the beam of light to the second folding prism; and the second folding prism is configured to deflect the second portion of the beam of light to the second pattern generating optical element. In some embodiments, the lens module further comprises a relay lens configured to focus the beam of light. In some embodiments, the relay lens is disposed between the diode module and a folding prism of the lens module. In some embodiments, the relay lens is disposed between a first folding prism of the lens module and the first pattern generating optical element. In some embodiments, the relay lens is further configured to deflect at least the first portion of the beam of light to the first pattern generating optical element. In some embodiments, the diode module further comprises focusing optics configured to focus the beam of light, the focusing optics being disposed between the laser diode and the lens module. In some embodiments, the first pattern generating optical element is disposed on a window structure of the intraoral scanner. In some embodiments, the intraoral scanner further comprises a plurality of cameras disposed in a camera portion of the intraoral scanner, wherein the camera portion of the intraoral scanner is disposed between the diode module and the lens module. In some embodiments, the beam of light emitted from the diode module is to pass between two or more cameras and is to be received by the lens module. In some embodiments, the intraoral scanner further comprises a first camera and a second camera, the first camera and the second camera being disposed in a distal portion of the intraoral scanner, wherein the lens module is disposed between the first camera and the second camera. In some embodiments, the intraoral scanner further comprises: a window coupled to the probe housing, wherein the lens module is disposed at or proximate to a first distal end of the window and the diode module is disposed at or proximate to a second distal end of the window. In some embodiments, the intraoral scanner further comprises a window coupled to the probe housing, wherein the window covers the lens module and does not cover the diode module. In some embodiments, the lens module is separated from the diode module by air, an optical fiber, or a light guide. In some embodiments,

In some embodiments, the intraoral scanner of one or more of FIGS. 6A-C includes: a probe housing disposed at a distal end of an elongate wand, the probe housing forming an interior volume; and a distributed projector disposed in the interior volume, the distributed projector comprising: a diode module configured to emit a first beam of light at a first wavelength; and a first prism component comprising: a first beam splitter configured to deflect a first portion of the first beam of light and transmit a second portion of the first beam of light; and a second beam splitter configured to deflect the second portion of the first beam of light. In some embodiments, the intraoral scanner further comprises: a lens module disposed at least a threshold distance from the diode module, the lens module comprising: a relay lens configured to focus the first portion of the first beam of light; a folding prism configured to deflect the first portion of the first beam of light; and a pattern generating optical element configured to generate first structured light based on the first portion of the first beam of light. In some embodiments, the diode module is further configured to emit a second beam of light at a second wavelength. In some embodiments, the intraoral scanner further comprises: a second prism component comprising: a third beam splitter configured to deflect a first portion of the second beam of light and transmit a second portion of the second beam of light; and a fourth beam splitter configured to deflect the second portion of the second beam of light. In some embodiments, the first beam splitter and the second beam splitter are further configured to transmit the second beam of light, wherein the first prism component further comprises: a third beam splitter configured to deflect a first portion of the second beam of light and transmit a second portion of the second beam of light; and a fourth beam splitter configured to deflect the second portion of the second beam of light. In some embodiments, the distributed projector is of an intraoral scanner, the distributed projector includes: a diode module comprising a laser diode configured to emit a beam of light; and a lens module disposed at least a threshold distance from the diode module, the lens module comprising: a folding prism configured to deflect at least a first portion of the beam of light; and a pattern generating optical element configured to generate, based on the at least a first portion of the beam of light, structured light to illuminate at least a portion of a mouth of a patient. In some embodiments, the diode module further comprises focusing optics configured to focus the beam of light. In some embodiments, the lens module further comprises a relay lens configured to focus the beam of light. In some embodiments,

In some embodiments, the intraoral scanner of one or more of FIGS. 6A-C includes: a probe housing disposed at a distal end of an elongate wand, the probe housing forming an interior volume; a non-distributed projector disposed in the interior volume; and a distributed projector disposed in the interior volume, the distributed projector comprising a diode module and a lens module. In some embodiments: the non-distributed projector comprising components configured to emit a first beam of light and generate first structured light; the components are less than a threshold distance from each other; the diode module is configured to emit a second beam of light; the lens module is configured to generate second structured light based on at least a portion of the second beam of light; and the lens module is disposed greater than the threshold distance from the diode module. In some embodiments, the lens module is disposed at a distal end of the probe housing. In some embodiments, the distal end of the probe housing has an angled tip that houses the lens module.

In some embodiments, the intraoral scanner of one or more of FIGS. 6A-C is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view of about 70 to about 90 degrees; and one or more non-object-facing imaging devices disposed within the interior volume in a rear portion of the probe housing, wherein the one or more non-object-facing imaging devices are configured to capture respective images via respective imaging axes that reflect to pass through the window structure, and wherein the one or more object-facing imaging devices have a second field of view of about 10 to about 30 degrees. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in the rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors. In some embodiments, the hybrid intraoral scanner further includes a fold mirror disposed at a distal end of the probe housing, wherein the fold mirror is configured to reflect the respective imaging axes and the respective projector axes to provide structured light 3D capture of a distal portion of a user mouth, and wherein the one or more non-object-facing imaging devices are oriented substantially parallel to the window structure. In some embodiments, the hybrid intraoral scanner further includes a prism disposed at a distal end of the probe housing, wherein the prism is configured to reflect the respective imaging axes and the respective projector axes to provide structured light 3D capture of a distal portion of a user mouth, wherein the prism has a fold angle of about 25 degrees to about 40 degrees, and wherein the one or more non-object-facing imaging devices are oriented substantially parallel to the window structure. In some embodiments, the hybrid intraoral scanner further includes a dichroic filter, wherein the one or more non-object-facing imaging devices comprise one or more upward-facing cameras that are oriented away from the window structure and an optical coherence tomography (OCT) channel that is oriented substantially parallel to the window structure, wherein the one or more upward-facing cameras are oriented upward and are configured to combine coaxially with a forward pointing OCT channel via the dichroic filter. In some embodiments, a first camera of the one or more object-facing imaging devices is configured to provide a viewfinder associated with moving the hybrid intraoral scanner. In some embodiments, In some embodiments, the one or more object-facing imaging devices and the one or more non-object-facing imaging devices are configured to provide imaging of one or more of a three-dimensional (3D) surface, subgingival, occlusal caries, proximal caries, or periodontal pockets. In some embodiments, the one or more object-facing imaging devices and the one or more non-object-facing imaging devices are configured to provide structured near-infrared (NIRI) images to provide shallow 3D viewing of hard tissue lesions. In some embodiments, the one or more object-facing imaging devices and the one or more object-facing projectors are configured to perform surface 3D capture. In some embodiments, the hybrid intraoral scanner further comprises an array of object-facing LEDs associated with two-dimensional color capture. In some embodiments, the one or more non-object-facing projectors comprise one or more of a rear NIRI structured light or NIRI collimated projector for subsurface 3D capture. In some embodiments, the one or more non-object-facing imaging devices comprise an OCT imaging channel coaxial configured for surface and sub-surface 3D structure capture. In some embodiments, the one or more non-object-facing imaging devices comprises an endoscopic OCT device inserted between the one or more object-facing imaging devices and the one or more object-facing projectors, wherein the endoscopic OCT device comprises a built-in rotating fold prism configured to provide transverse line scanning. In some embodiments, image data from the one or more non-object-facing imaging devices and the one or more object-facing imaging devices is to be combined to provide a third field of view that is greater than at least one of the first field of view or the second field of view. In some embodiments, the hybrid intraoral scanner further comprises a collimator, a scan mirror, and a fold mirror, wherein the one or more non-object-facing imaging devices comprise an OCT measuring arm fiber configured to provide light that is to pass through the collimator, reflect off the scan mirror, and reflect off the fold mirror to pass through the window structure.

In some embodiments, the intraoral scanner of one or more of FIGS. 6A-C is used with a method that includes: identifying first image data from one or more object-facing imaging devices at a first field of view of about 70 to about 90 degrees, the one or more object-facing imaging devices being disposed within an interior volume formed by a probe housing and a window structure of a hybrid intraoral scanner, the one or more object-facing imaging devices being disposed proximate the window structure; identifying second image data from one or more non-object-facing imaging devices at a second field of view of about 10 to about 30 degrees, the one or more non-object-facing imaging devices being disposed within the interior volume in a rear portion of the probe housing; and combining the first image data and the second image data. In some embodiments, the hybrid intraoral scanner further comprises: one or more object-facing projectors disposed within the interior volume proximate the window structure, wherein the one or more object-facing projectors provide corresponding light via corresponding projector axes that directly pass through the window structure; and one or more non-object-facing projectors disposed within the interior volume in a rear portion of the probe housing, wherein the one or more non-object-facing projectors provide respective light via respective projector axes that reflect to pass through the window structure. In some embodiments: the respective light is provided via a pattern generating optical element prior to being reflected to pass through the window structure; and the one or more object-facing devices and the one or more object-facing projectors are closer to a distal tip of the probe housing than the one or more non-object-facing imaging devices and the one or more non-object-facing projectors.

In some embodiments, the intraoral scanner of one or more of FIGS. 6A-C is a hybrid intraoral scanner that includes: a probe housing disposed at a distal end of an elongate wand; a window structure coupled to the probe housing, the window structure and the probe housing forming an interior volume; and one or more object-facing imaging devices disposed within the interior volume proximate the window structure, wherein the one or more object-facing imaging devices are configured to capture corresponding images via corresponding imaging axes that directly pass through the window structure, and wherein the one or more object-facing imaging devices have a first field of view; and an optical coherence tomography (OCT) imaging channel disposed within the interior volume in a rear portion of the probe housing, wherein the OCT imaging channel is configured to capture respective images via a respective imaging axis that reflects to pass through the window structure, and wherein the OCT imaging channel has a second field of view that is different from the first field of view. In some embodiments, the hybrid intraoral scanner further includes one or more object-facing projectors disposed within the interior volume proximate the window structure, wherein the one or more object-facing projectors provide corresponding light via corresponding projector axes that directly pass through the window structure. In some embodiments, the one or more object-facing devices and the one or more object-facing projectors are closer to a distal tip of the probe housing than the OCT imaging channel.

An intraoral scanner 300 (e.g., multi-camera scanner) may include a camera 310A that has a first static focal distance (e.g., about 3 mm to about 7 mm, about 5 mm, etc.), a camera 310B that has a second static focal distance (e.g., about 8 mm to about 12 mm, about 10 mm, etc.), and a camera 310C that has a third static focal distance (e.g., about 13 mm to about 17 mm, about 15 mm, etc.), where the first, second, and third static focal distances are different from each other.

The intraoral scanner 300 may have a processing device 610 that receives images captured by the cameras 310. The processing device may determine more sharp (e.g., more in-focus, less blurry) portions of the images from the different cameras (or same camera in different time) and combine the more sharp portions of the images to generate a combined image. The processing device may cause, based on the combined image, an action (e.g., caries detection, margin line detection, interproximal area detection, texture mapping of a two-dimensional image on a three-dimensional model, displaying image data of portions of the mouth of the patient via a review tool graphical user interface, etc.).

In some embodiments, the intraoral scanner 300 has a different best object focus distance for each camera 310 (e.g., intraoral scanner 300 has variety of best object focus).

In some embodiments, the intraoral scanner 300 (e.g., processing device 601) captures many (e.g., thousands) of white light images (e.g., provide light from white LEDs 360A on the mouth of the user while capturing images) and NIR images (e.g., provide NIR images from NIR LEDs 360B on the mouth of the user while capturing images) during the scan (e.g., and even more multi-structure light images by providing structured patterns from projectors 320 on the mouth of the user while capturing images).

Conventional systems use a single focal distance for all cameras. Intraoral scanner 300 uses cameras 310 that each have a different static focal distance (e.g., different “best object focus distance”) instead of using a single focal distance for all cameras (e.g., single best object focus distance”). This provides better sharpness in texture mapping and review tool images (e.g., for both white light and NIR). Using a variety of static focal distances in the intraoral scanner 300 provides a greater variety of best object focus to choose from for every image patch (e.g., there are images captured by a camera 310A that has a first static focal distance, images captured by a camera 310B that has a second static focal distance, and images captured by a camera 310C that has a third static focal distance to choose from instead of all images being at the same focal distance).

In some embodiments, a processing device 601 may generate a 3D model of an object 603 (e.g., dental arch, tooth, etc.), identify 2D images of the object 603, and perform texture mapping to overlay the 2D images on the 3D model. In some embodiments, in texture mapping, an image patch (e.g., small part of an image that corresponds to a face) is selected for every face in the surface (e.g., each face uses a different image patch). A face may be a portion of a surface of an object 603. A larger variety of static focal distances allows selecting sharper image patches for every face.

In some embodiments, an object has different distances 607 from the intraoral scanner 300. A top of a tooth may have a distance 607A from the intraoral scanner 300 and a crevice between teeth may have a distance 607B from the intraoral scanner 300.

In a review tool, an image that is sharper may be synthesized by choosing the sharpest image patch for every area of the image and creating a single image from all the patches (e.g., a single image can be chosen which is better than the rest). One single image may not be sharp in every portion of the image.

If two or more cameras 310 (e.g., each camera 310) in an intraoral scanner 300 uses a different static focal distance than the other camera(s) 310, then the probability of having few images seeing the same face with different sharpness levels is higher. Sharper images (e.g., portion of image covering a single face) may be selected for the texture mapping image (e.g., to overlay on the 3D model via texture mapping).

Images with better sharpness could be synthesized for a review tool. The review tool may display the 3D model via a user interface and upon receiving user input (e.g., cursor pointing) associated with a portion of the 3D model, the user interface may display a combined image (e.g., synthesized image) corresponding to the portion of the 3D model.

Conventional systems use the same focal distance for each camera. Conventionally the chance of seeing the same face from the same angle but with a large variety of sharpness is low. Images provided by conventional systems have less sharp images compared to combined images of the intraoral scanner 300 of the present disclosure.

To acquire images of a full or partial jaw (e.g., dental arch), the user is to move the intraoral scanner 300 (e.g., wand). The usual movement (e.g., wand movement direction 605) of the intraoral scanner 300 (e.g., wand) is forward or backward movement substantially parallel to a surface of the tooth so the distance of the intraoral scanner 300 (e.g., wand) from the tooth is changing slowly. To choose the sharpest image for every part of the tooth, a different static focal distance (e.g., different focal length) for two or more of the cameras 310 (e.g., each camera) is used to have better chance to choose sharper image for every face (e.g., for texture mapping). Similar images with different focal lengths can be used to synthesize a sharper image for the review tool (e.g., by using few different images).

By using a camera 310A that has a first static focal distance (e.g., 5 mm), a camera 310B that has a second static focal distance (e.g., 10 mm), and a camera 310C that has a third static focal distance (e.g., 15 mm), a sharper image can be created for different portions of a tooth compared to conventional systems.

In some embodiments, different static focal distances (e.g., best object focus) on the same area in different images could be used to have better image resolution.

In some embodiments, different static focal distances (e.g., a few best object focuses) can be used to accurately detect structured patterns of light provided by projectors 320 on portions of objects 603 at different distances.

In some embodiments, the intraoral scanner 300 can provided better focus for inter-proximal detection.

In some embodiments, the intraoral scanner 300 can perform better carries detection by using different static focal distances (e.g., a variety of “best object focus distance”) for both white light images (e.g., capturing images while providing white light via white LED 360A) and NIR images (e.g., capturing images while providing NIR light via NIR LED 360B).

In some embodiments, the intraoral scanner 300 can perform better feature detection by using different static focal distances (e.g., a variety of best focus distances).

In some embodiments, 2D images (e.g., white light images and/or NIR images) can be better mapped onto a 3D model by using different static focal distances (e.g., a variety of best focus distances).

In some embodiments, margin line creation and detection can be better performed by using different static focal distances (e.g., a variety of best focus distances).

In some embodiments, each camera 310 has a static focal distance of a single focal distance that does not change (e.g., is not an auto-focus camera).

In some embodiments, each camera 310 has two static focal distances of a first focal distance and a second focal distance, where each camera 310 cannot use a different focal distance other than the corresponding first focal distance and the corresponding second focal distance of that camera 310.

Cameras 310 may be structured light cameras. Since cameras 310 have a static focal distance, cameras 310 have a shorter height than autofocus cameras to reduce the height of the intraoral scanner 300.

Processing device 601 of intraoral scanner 300 can produce combined images that have sharp features (e.g., high resolution). For example, margin line, interproximal areas, incisal edge, etc. may be a sharp feature. Cameras 310 may produce color images (e.g., for color texture mapping of 2D images on a 3D model).

Intraoral scanner 300 may have a distal pair of cameras 310A that have a first static focal distance, a rear pair of cameras 310C that have a third static focal distance, and a mid-pair of cameras 310B that have a second static focal distance and are located between the distal pair and the rear pair, where the first, second, and third static focal distances are different from each other. In some embodiments, the two cameras 310 in a pair of cameras (e.g., distal pair, mid pair, rear pair) have different static focal distances.

In some embodiments, the intraoral scanner 300 has projectors 320 that are tuned to match the closest cameras 310 (e.g., structured pattern of light is to substantially match the static focal distance of the closest cameras 310).

In some embodiments, images are captured by cameras 310 of an intraoral scanner 300 and portions of the images are identified (e.g., labeled, manually labeled by a user) as a more sharp (e.g., more in-focus, less blurry, more edges, more contrast, etc.) portion of the images. A processing device 601 may train a machine learning model with training data including training input including the images and target output of an indication of the sharper portions of the images to generate a trained machine learning model. Then, a processing device 601 may provide new images captured by cameras 310 of an intraoral scanner 300 to the trained machine learning model and receive, from the machine learning model, output associated with predictive data. The processing device 601 may predict the more sharp (e.g., more in-focus, less blurry, more edges, more contrast, etc.) portions of the images based on the predictive data.

In some embodiments, a height map (e.g., distance from the camera 310 to the teeth) is determined based on scanning of the mouth (e.g., by performing triangulation based on the images from the cameras 310). The structured pattern from a projector 320 may be viewed from two cameras 310 and a correspondence algorithm may be used to identify the heigh of the spots of the structured pattern (e.g., triangulate the spots in 3D space). In some embodiments, the triangulation between the projector 320 and the camera 310 (e.g., the structured pattern of light provided from the projector 320 and the image of the structured pattern of light captured by the camera 310) may be used to determine the distance at each point of the image.

The intraoral scanner may alternate between capturing images responsive to white light being provided by the white LEDs 360A and structured pattern of light being provided by the projectors 320 to determine a height map.

In some embodiments, the intraoral scanner 300 (e.g., processing device 601) causes the projectors 320 to provide structured pattern of light (e.g., spots) and causes the cameras 310 to capture images of the structured pattern to generate a point cloud. The intraoral scanner 300 (e.g., processing device 601) then causes the projectors 320 to stop providing the structured pattern of light, causes the LEDs 360 (e.g., white LEDs 360A) to provide light (e.g., white light), and causes the cameras 310 to capture images (e.g., white light images). The processing device 601 then combines the point cloud (e.g., 3D model) with the white light images (e.g., 2D images) at the same position and orientation to generate 3D data (e.g., overlay the white images on the point cloud). This can also be done with NIR images captured when providing NIR light via NIR LEDs 360B.

These processes can be used with inter-proximal detection and/or margin line creation and detection. Inter-proximal may refer to the space between teeth (e.g., crevice between teeth). Margin line may be below the gum line (e.g., lift the gum tissue at the tooth to see the margin line between the gum and the tooth before the gum falls back down). Margin line may be used for a crown procedure where the tooth is ground down and cut under the margin line to create a lip (e.g., under the gum tissue) for the crown to fit over.

In some embodiments, the intraoral scanner 300 has six cameras 310. In some embodiments, the intraoral scanner 300 has more or less than six cameras 310.

In some embodiments, there is more than one (e.g., two, three, four, five, six, etc.) static focal distance among the cameras 310 in the intraoral scanner 300.

In some embodiments, different static focal distance assists with spot detection for the multi-structure light (MSL) provided by the projectors 320 and may improve the 3D surface.

FIGS. 6B-C are flow diagrams of methods 600B-C associated with intraoral scanners (e.g., intraoral scanner 300 of FIG. 6A), according to certain embodiments. In some embodiments, one or more of methods 600B-C is performed by processing logic that includes hardware (e.g., circuitry, dedicated logic, programmable logic, microcode, processing device 601, etc.), software (such as instructions run on a processing device 601, a general-purpose computer system, or a dedicated machine), firmware, microcode, or a combination thereof. In some embodiments, one or more of methods 600B-C is performed, at least in part, by a processing device 601 (e.g., of an intraoral scanner 300). In some embodiments, a non-transitory machine-readable storage medium stores instructions that when executed by a processing device 601, cause the processing device 601 to perform one or more operation of one or more of methods 600B-C.

For simplicity of explanation, methods 600B-C are depicted and described as a series of operations. However, operations in accordance with this disclosure can occur in various orders and/or concurrently and with other operations not presented and described herein. Furthermore, in some embodiments, not all illustrated operations are performed to implement methods 600B-C in accordance with the disclosed subject matter. In addition, those skilled in the art will understand and appreciate that methods 600B-C could alternatively be represented as a series of interrelated states via a state diagram or events.

Referring to FIG. 6B, method 600B may be performed by a processing device 601 of an intraoral scanner 300 (e.g., of FIG. 6A) that includes a camera 310A that has a first static focal distance (e.g., about 3 mm to about 7 mm, about 5 mm, etc.), a camera 310B that has a second static focal distance (e.g., about 8 mm to about 12 mm, about 10 mm, etc.), and a camera 310C that has a third static focal distance (e.g., about 13 mm to about 17 mm, about 15 mm, etc.), where the first, second, and third static focal distances are different from each other.

At block 602, processing logic receives a first image of an object 603 from camera 310A.

At block 604, processing logic determines a more sharp portion (e.g., a more in-focus portion) of the first image and a less sharp portion (e.g., less in-focus portion, a blurry portion) of the first image, the less sharp portion corresponding to a region of the object 603.

At block 606, for the less sharp portion of the first image, the processing logic determines a more sharp portion of a second image corresponding to the region of the object, the second image being from a camera 310B of the intraoral scanner. In some embodiments, the first image of the object being captured by camera 310A and the second image of the object being captured by camera 310B in a similar location, in a similar angle, and at a similar brightness level. In some embodiments, camera 310A and camera 310B capture images of the mouth of the user responsive to a light source of the intraoral scanner 300 providing white light or near infrared (NIR) light into the mouth of the user.

At block 608, processing logic generates a combined image using the more sharp portion of the first image and the more sharp portion of the second image. In some embodiments, the combined image has an increased sharpness than the sharp portion of the first image and the sharp portion of the second image.

At block 610, processing logic causes, based on the combined image, an action associated with a mouth of a user. In some embodiments, the action associated with the mouth of the user includes one or more of caries detection (e.g., machine learning caries detection), margin line detection, interproximal area detection, texture mapping of a two-dimensional image on a three-dimensional model, or displaying image data of portions of the mouth of the user via a review tool graphical user interface (e.g., via a viewfinder, review tool, etc.).

In some embodiments, to generate the combined image, the processing device 601 is to smooth the more sharp portion of the first image and the more sharp portion of the second image.

In some embodiments, the processing device 601 is further configured to receive a third image of the object from a camera 310C and the processing device 601 is to generate the combined image further based on the third image. In some embodiments, camera 310A (e.g., distal camera) is proximate a distal end of a tip of the intraoral scanner, camera 310C (e.g., rear camera) is further from the distal end of the tip of the intraoral scanner than camera 310A, and camera 310B is disposed between camera 310A and camera 310C. The first static focal distance of camera 310A is less than the second static focal distance of camera 310B. The second static focal distance of camera 310B is less than the third static focal distance of camera 310C. The first static focal distance of camera 310A is less than the third static focal distance of camera 310C.

Referring to FIG. 6C, method 600C may be performed by a processing device 601 of an intraoral scanner 300 (e.g., of FIG. 6A) that includes a camera 310A that has a first static focal distance (e.g., about 3 mm to about 7 mm, about 5 mm, etc.), a camera 310B that has a second static focal distance (e.g., about 8 mm to about 12 mm, about 10 mm, etc.), and a camera 310C that has a third static focal distance (e.g., about 13 mm to about 17 mm, about 15 mm, etc.), where the first, second, and third static focal distances are different from each other.

At block 620, processing logic receives a first image of an object from camera 310A.

At block 622, processing logic receives a second image of the object from camera 310B. In some embodiments, the first image of the object being captured by camera 310A and the second image of the object being captured by camera 310B in a similar location, in a similar angle, and at a similar brightness level. In some embodiments, camera 310A and camera 310B capture images of the mouth of the user responsive to a light source of the intraoral scanner 300 providing white light or near infrared (NIR) light into the mouth of the user.

At block 624, processing logic determines a first sharp portion (e.g., a more in-focus portion) of the first image corresponding to a region of the object.

At block 626, processing logic determines a second sharp portion (e.g., a more in-focus portion) of the second image corresponding to the region of the object.

At block 628, processing logic generates a combined image using the first sharp portion of the first image and the second sharp portion of the second image. In some embodiments, the combined image has an increased sharpness than the sharp portion of the first image and the sharp portion of the second image.

At block 630, processing logic causes, based on the combined image, an action associated with a mouth of a user. In some embodiments, the action associated with the mouth of the user includes one or more of caries detection (e.g., machine learning caries detection), margin line detection, interproximal area detection, texture mapping of a two-dimensional image on a three-dimensional model, or displaying image data of portions of the mouth of the user via a review tool graphical user interface (e.g., via a viewfinder, review tool, etc.).

In some embodiments, to generate the combined image, the processing device 601 is to smooth the more sharp portion of the first image and the more sharp portion of the second image.

In some embodiments, the processing device 601 is further configured to receive a third image of the object from a camera 310C and the processing device 601 is to generate the combined image further based on the third image. In some embodiments, camera 310A (e.g., distal camera) is proximate a distal end of a tip of the intraoral scanner, camera 310C (e.g., rear camera) is further from the distal end of the tip of the intraoral scanner than camera 310A, and camera 310B is disposed between camera 310A and camera 310C. The first static focal distance of camera 310A is less than the second static focal distance of camera 310B. The second static focal distance of camera 310B is less than the third static focal distance of camera 310C. The first static focal distance of camera 310A is less than the third static focal distance of camera 310C.

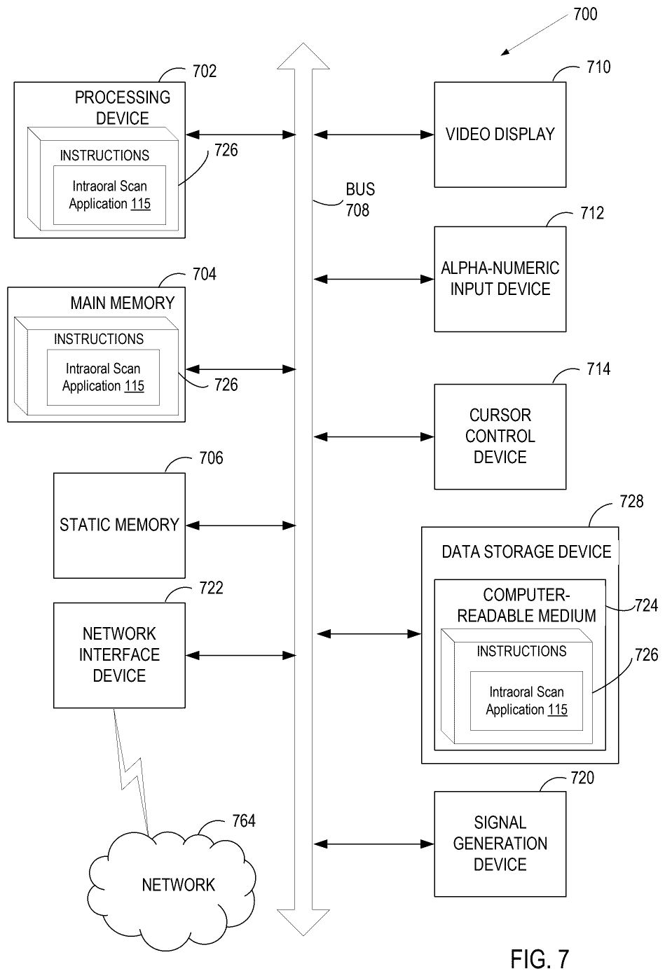

FIG. 7 illustrates a block diagram of an example computing device 700, according to certain embodiments. In some embodiments, FIG. 7 illustrates a diagrammatic representation of a machine in the example form of a computing device 700 within which a set of instructions, for causing the machine to perform any one or more of the methodologies discussed herein (e.g., methods 600B-C), may be executed. In some embodiments, the machine may be connected (e.g., networked) to other machines in a Local Area Network (LAN), an intranet, an extranet, or the Internet. The computing device 700 may correspond, for example, to computing device 105 and/or computing device 106 of FIG. 1. The computing device 700 may correspond, for example, to processing device 601. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 700 includes a processing device 702, a main memory 704 (e.g., read-only memory (ROM), flash memory, dynamic random access memory (DRAM) such as synchronous DRAM (SDRAM), etc.), a static memory 706 (e.g., flash memory, static random access memory (SRAM), etc.), and a secondary memory (e.g., a data storage device 728), which communicate with each other via a bus 708.

Processing device 702 represents one or more general-purpose processors such as a microprocessor, central processing unit, or the like. More particularly, the processing device 702 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. Processing device 702 is configured to execute the processing logic (instructions 726) for performing operations and steps discussed herein.

The computing device 700 may further include a network interface device 722 for communicating with a network 764. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse), and a signal generation device 720 (e.g., a speaker).

The data storage device 728 may include a machine-readable storage medium (or more specifically a non-transitory computer-readable storage medium) 724 on which is stored one or more sets of instructions 726 embodying any one or more of the methodologies or functions described herein, such as instructions for intraoral scan application 715, which may correspond to intraoral scan application 115 of FIG. 1. A non-transitory storage medium refers to a storage medium other than a carrier wave. The instructions 726 may also reside, completely or at least partially, within the main memory 704 and/or within the processing device 702 during execution thereof by the computing device 700, the main memory 704 and the processing device 702 also constituting computer-readable storage media.

The computer-readable storage medium 724 may also be used to store intraoral scan application 115, which may include one or more machine learning modules, and which may perform the operations described herein above. The computer readable storage medium 724 may also store a software library containing methods for the intraoral scan application 115. While the computer-readable storage medium 724 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

In some embodiments, the methods, components, and features described herein are implemented by discrete hardware components or are integrated in the functionality of other hardware components such as ASICs, FPGAs, DSPs, or similar devices. In some embodiments, the methods, components, and features are implemented by firmware modules or functional circuitry within hardware devices. In some embodiments, the methods, components, and features are implemented in any combination of hardware devices and computer program components, or in computer programs.

Unless specifically stated otherwise, terms such as “transmitting,” “receiving,” “identifying,” “determining,” “generating,” “providing,” “obtaining,” “causing,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. In some embodiments, the terms “first,” “second,” “third,” “fourth,” etc. as used herein are meant as labels to distinguish among different elements and do not have an ordinal meaning according to their numerical designation.

Examples described herein also relate to an apparatus for performing the methods described herein. In some embodiments, this apparatus is specially constructed for performing the methods described herein or includes a general-purpose computer system selectively programmed by a computer program stored in the computer system. Such a computer program is stored in a computer-readable tangible storage medium.

Some of the methods and illustrative examples described herein are not inherently related to any particular computer or other apparatus. In some embodiments, various general-purpose systems are used in accordance with the teachings described herein. In some embodiments, a more specialized apparatus is constructed to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. An intraoral scanner comprising:
a probe housing disposed at a distal end of an elongate wand;
a window structure coupled to the probe housing, the window structure and the probe housing forming an interior volume, wherein the window structure has a concave transverse cross section; and
a plurality of optical components disposed within the interior volume, wherein the plurality of optical components comprise a first camera having a first orientation and a second camera having a second orientation that is different than the first orientation, wherein the first camera and the second camera are to capture images of dental sites.

2. The intraoral scanner of claim 1, wherein the plurality of optical components comprises a first pattern projector, a second pattern projector, and a camera disposed between the first pattern projector and the second pattern projector.

3. The intraoral scanner of claim 2, wherein the camera, the first pattern projector and the second pattern projector are disposed within 5 millimeters of a foremost end of the probe housing.

4. The intraoral scanner of claim 2, wherein each of the first pattern projector and the second pattern projector has a diameter of less than 4 millimeters and a height of less than 4.5 millimeters.

5. The intraoral scanner of claim 2, wherein each of the first pattern projector and the second pattern projector has a diameter of about 2 millimeters and a height of about 3 millimeters.

6. The intraoral scanner of claim 1, wherein the probe housing has a height of less than 10 millimeters and a width of less than 20 millimeters.

7. The intraoral scanner of claim 1, wherein the probe housing has a height of about 7.5 to about 8.5 millimeters and a width of about 16 to about 17 millimeters.

8. The intraoral scanner of claim 1, wherein:
the first orientation of the first camera and the second orientation of the second camera are configured to cause about 20% to about 40% overlap of image capture between the first camera and the second camera.

9. The intraoral scanner of claim 1, wherein the first camera and the second camera are to capture images, wherein the images are to be used to perform model building via at least one of a correspondence algorithm or a trained machine learning model.

10. The intraoral scanner of claim 1, wherein one or more of the plurality of optical components are configured to capture images of rearmost teeth in a mouth of a patient.

11. The intraoral scanner of claim 1, wherein the first camera is to perform image capturing in the first orientation of a first side of the dental sites and the second camera is to perform image capturing in the second orientation of a second side of the dental sites to perform teeth wrapping imaging.

12. The intraoral scanner of claim 1, wherein a first central axis of the first camera is orthogonal to a first portion of the window structure, and wherein a second central axis of the second camera is orthogonal to a second portion of the window structure.

13. An intraoral scanner comprising:
a probe housing disposed at a distal end of an elongate wand, the probe housing forming an opening;
a window structure coupled to the probe housing, the window structure covering the opening, the window structure and the probe housing forming an interior volume; and
a plurality of optical components disposed within the interior volume;
a metal structure disposed in the interior volume, the metal structure partially surrounding one or more of the plurality of optical components, the plurality of optical components being bonded directly to at least one of the window structure or the metal structure.

14. The intraoral scanner of claim 13, wherein the plurality of optical components comprises cameras and projectors.

15. The intraoral scanner of claim 13, wherein the plurality of optical components are bonded directly to the at least one of the window structure or the metal structure to provide drift-free retention of the plurality of optical components.

16. The intraoral scanner of claim 13, wherein the plurality of optical components are bonded directly to the window structure via adhesive that is optically permeable.

17. An intraoral scanner comprising:
a probe housing disposed at a distal end of an elongate wand, the probe housing forming an opening;
a window structure coupled to the probe housing, the window structure covering the opening, the window structure and the probe housing forming an interior volume; and
a plurality of optical components disposed within the interior volume, wherein the window structure comprises a first distal portion disposed at a first orientation and a second distal portion disposed at a second orientation that is at an angle to the first orientation, wherein the window structure is configured to block back reflection crosstalk by the plurality of optical components.

18. The intraoral scanner of claim 17, wherein the plurality of optical components comprises cameras and projectors.

19. The intraoral scanner of claim 17, wherein the window structure is a single piece of glass formed via glass molding.

20. The intraoral scanner of claim 17, wherein the probe housing is wrapped by a heat sink metal structure that is in thermal contact with the probe housing.

21. The intraoral scanner of claim 17, wherein a heating element is disposed between two or more of the plurality of optical components to heat the intraoral scanner.

22. The intraoral scanner of claim 17, further comprising a sleeve that includes an optical window, wherein the sleeve is configured to be removably disposed over the probe housing, wherein the optical window is configured to substantially align with the window structure.

23. The intraoral scanner of claim 22, wherein the optical window of the sleeve comprises a first portion that approximately aligns with the first distal portion of the window structure, a second portion that approximately aligns with the second distal portion of the window structure, and a third portion that approximately aligns with a third portion of the window structure, the third portion of the window structure being disposed between the first distal portion and the second distal portion.

24. The intraoral scanner of claim 17, wherein the window structure has a concave transverse cross section.

25. The intraoral scanner of claim 17, wherein a first optical component of the plurality of optical components has a first axis that is orthogonal to the first distal portion of the window structure, wherein a second optical component of the plurality of optical components has a second axis that is orthogonal to the second distal portion of the window structure, and wherein a third optical component of the plurality of optical components has a third axis that is orthogonal to a third portion of the window structure, the third portion of the window structure being disposed between the first distal portion and the second distal portion.

26. The intraoral scanner of claim 25, wherein the first optical component comprises a first structured light projector, wherein the second optical component comprises a camera, and wherein the third optical component comprises a second structured light projector.

27. The intraoral scanner of claim 25, wherein the first optical component comprises a first camera, wherein the second optical component comprises a structured light projector, and wherein the third optical component comprises a second camera.

28-87. (canceled)
